(19) United States
(12) Patent Application Publication
(10) Pub. No.: US 2019/0142974 A1
(43) Pub. Date: May 16, 2019

(54) METHODS AND COMPOSITIONS FOR ATTENUATING ANTI-VIRAL TRANSFER VECTOR IGM RESPONSES

(71) Applicant: Selecta Biosciences, Inc., Watertown, MA (US)

(72) Inventors: Petr Ilyinskii, Cambridge, MA (US); Christopher J. Royma, Newton, MA (US); Takashi Kei Kishimoto, Lexington, MA (US)

(21) Appl. No.: 16/159,166

(22) Filed: Oct. 12, 2018

Related U.S. Application Data
(60) Provisional application No. 62/572,297, filed on Oct. 13, 2017.

(51) Int. Cl. A61K 48/00; C12N 15/85; C12N 15/113; A61K 9/51; A61K 38/16; A61K 31/436; C07K 16/42

(52) U.S. Cl. CPC A61K 48/0066; C12N 15/85; C12N 15/113; A61K 48/0008; A61P 37/06; A61K 9/51; A61K 38/162; A61K 31/436; C07K 16/4291; A61K 48/0083

(57) ABSTRACT

Provided herein are methods and related compositions or kits for administering viral transfer vectors in combination with synthetic nanocarriers comprising an immunosuppressant and an anti-IgM agent.

### RELATED APPLICATIONS

This application claims the benefit under 35 U.S.C. § 119 of U.S. provisional application 62/572,297, filed Oct. 13, 2017, the entire contents of which is incorporated herein by reference.

### FIELD OF THE INVENTION

The invention relates to methods and related compositions for administering viral transfer vectors with synthetic nanocarriers comprising an immunosuppressant and an anti-IgM agent to a subject. Preferably, the methods and compositions are for reducing or preventing IgM responses against the viral transfer vector.

### SUMMARY OF THE INVENTION

In one aspect, a method comprising establishing an anti-viral transfer vector attenuated response in a subject by concomitant administration of a viral transfer vector, synthetic nanocarriers comprising an immunosuppressant, and an anti-IgM agent, to the subject is provided.

In one embodiment of any one of the methods provided herein the anti-viral transfer vector attenuated response is an IgM response against the viral transfer vector.

In another aspect, a method comprising escalating transgene expression of a viral transfer vector in a subject by repeatedly, concomitantly administering to the subject a viral transfer vector, synthetic nanocarriers comprising an immunosuppressant and an anti-IgM agent is provided.

In one embodiment of any one of the methods provided herein, the concomitant administration of the viral transfer vector, synthetic nanocarriers comprising an immunosuppressant and/or anti-IgM agent is repeated.

In one embodiment of any one of the methods, compositions or kits provided, the viral transfer vector is any one of the viral transfer vectors provided herein such as any one of such vectors defined in any one of the claims.

In one embodiment of any one of the methods, compositions or kits provided, the synthetic nanocarriers are any one of the synthetic nanocarriers provided herein such as any one of such synthetic nanocarriers defined in any one of the claims.

In one embodiment of any one of the methods, compositions or kits provided, the anti-IgM agent is an IgM antagonist antibody. IgM antagonist antibodies or antigen-binding fragments thereof specifically bind to CD10, CD19, CD20, CD22, CD27, CD34, CD40, CD79a, CD79b, CD123, CD179b, FLT-3, ROR1, BR3, BAFF, or B7RP-1. In one embodiment, the IgM antagonist antibody or antigen-binding fragment thereof is any one of the CD10, CD19, CD20, CD22, CD27, CD34, CD40, CD79a, CD79b, CD123, CD179b, FLT-3, ROR1, BR3, BAFF, or B7RP-1 antibodies or antigen-binding fragments thereof provided herein such as any one of such CD10, CD19, CD20, CD22, CD27, CD34, CD40, CD79a, CD79b, CD123, CD179b, FLT-3, ROR1, BR3, BAFF, or B7RP-1 antibodies or antigen-binding fragments thereof defined in any one of the claims.

In one embodiment of any one of the methods, compositions or kits provided, the IgM antagonist antibody is an anti-BAFF antibody or antigen-binding fragment thereof. In one embodiment, the anti-BAFF antibody or antigen-binding fragment thereof is any one of the anti-BAFF antibodies or antigen-binding fragments thereof provided herein such as any one of such anti-BAFF antibodies or antigen-binding fragments thereof defined in any one of the claims.

In one embodiment of any one of the methods, compositions or kits provided, the anti IgM agent is an anti-BAFF agent. In one embodiment, the anti-BAFF agent is any one of the anti-BAFF agents provided herein such as any one of such anti-BAFF agents defined in any one of the claims.

In one embodiment of any one of the methods, compositions or kits provided, the anti IgM agent is an IL-21 modulating agent, e.g., an IL-21 antagonist or IL-21 receptor antagonist. In one embodiment, the IL-21 modulating agent is any one of the IL-21 modulating agents provided herein such as any one of such IL-21 modulating agents defined in any one of the claims.

In one embodiment of any one of the methods, compositions or kits provided, the anti IgM agent is a tyrosine kinase inhibitor, e.g., a Syk inhibitor, a BTK inhibitor, or a SRC protein tyrosine kinase inhibitor. In one embodiment, the tyrosine kinase inhibitor is any one of the tyrosine kinase inhibitors provided herein such as any one of such tyrosine kinase inhibitors defined in any one of the claims. In one embodiment of any one of the methods, compositions or kits provided, the tyrosine kinase inhibitor is a Syk inhibitor. In one embodiment, the Syk kinase inhibitor is any one of the Syk inhibitors provided herein such as any one of such Syk inhibitors defined in any one of the claims. In one embodiment of any one of the methods, compositions or kits provided, the tyrosine kinase inhibitor is a BTK inhibitor. In one embodiment, the BTK kinase inhibitor is any one of the BTK inhibitors provided herein such as any one of such BTK inhibitors defined in any one of the claims. In one embodiment of any one of the methods, compositions or kits provided, the tyrosine kinase inhibitor is a SRC protein tyrosine kinase inhibitor. In one embodiment, the SRC protein tyrosine kinase inhibitor is any one of the SRC protein tyrosine kinase inhibitors provided herein such as any one of such SRC protein tyrosine kinase inhibitors defined in any one of the claims.

In one embodiment of any one of the methods, compositions or kits provided, the anti IgM agent is a PI3K inhibitor. In one embodiment, the PI3K inhibitor is any one of the PI3K inhibitors provided herein such as any one of such PI3K inhibitors defined in any one of the claims.

In one embodiment of any one of the methods, compositions or kits provided, the anti IgM agent is a PKC inhibitor. In one embodiment, the PKC inhibitor is any one of the PKC inhibitors provided herein such as any one of such PKC inhibitors defined in any one of the claims.

In one embodiment of any one of the methods, compositions or kits provided, the anti IgM agent is a APRIL antagonist. In one embodiment, the APRIL antagonist is any one of the APRIL antagonists provided herein such as any one of such APRIL antagonists defined in any one of the claims.

In one embodiment of any one of the methods, compositions or kits provided, the anti IgM agent is a tetracycline. In one embodiment, the tetracycline is any one of the tetracyclines provided herein such as any one of such tetracyclines defined in any one of the claims.

In one embodiment of any one of the methods, compositions or kits provided, the anti IgM agent is mizoribine or tofacitinib.

In another aspect, compositions are provided, such as kits, comprising any one of the viral transfer vectors provided herein, any one of the synthetic nanocarriers provided herein and any one of the anti-IgM agents provided herein.

In another aspect, a kit comprising any one of the compositions or combinations of compositions provided herein is provided. In one embodiment of any one of the kits provided, the kit further comprises instructions for use. In one embodiment of any one of the kits provided, the instructions for use comprises instructions for carrying out any one of the methods provided herein.

In another aspect a method or composition as described in any one of the Examples is provided.

In another aspect, any one of the compositions is for use in any one of the methods provided.

In another aspect, any one of the method or compositions is for use in treating any one of the diseases or conditions described herein. In another aspect, any one of the methods or compositions is for use in attenuating an anti-viral transfer vector response (e.g., IgM response), establishing an attenuated anti-viral transfer vector response (e.g., IgM response), escalating transgene expression and/or for repeated administration of a viral transfer vector.

In another aspect, a method of administering any combination of the agents of the Examples is provided. In another aspect, a composition or kit comprising any one of these combinations of agents is also provided.

In one embodiment of any one of the methods, compositions or kits, the method, composition or kit is for attenuating an IgM response in addition to another immune response, such as an IgG response, humoral or cellular immune response.

In one embodiment of any one of the methods, compositions or kits, the method, composition or kit is for attenuating an IgM response in addition to increasing transgene expression.

In one embodiment of any one of the methods, compositions or kits, the method, composition or kit is for attenuating an IgM response in addition to another immune response, such as an IgG response, humoral or cellular immune response, as well as increasing transgene expression.

### BRIEF DESCRIPTION OF THE FIGURES

FIG. 1 shows serum anti-AAV IgM levels in mice 5, 9, 12, 16, and 21 days following administration of the indicated treatment (adeno-associated viral vector encoding secreted alkaline phosphatase (AAV-SEAP) alone, in combination with synthetic nanocarriers comprising rapamycin (AAV-SEAP+SVP[RAPA]), or in combination with anti-BAFF (AAV-SEAP+SVP[RAPA]+anti-BAFF)). Each treatment group contained six mice.

FIG. 2 shows SEAP expression level, measured using chemiluminescence, 5, 9, 12, and 16 days after administration of treatment from the same mice as described in FIG. 1.

FIG. 3 shows that both BAFF and APRIL support B cell survival and differentiation. Antibody to BAFF or a dual BAFF/APRIL inhibitor TACI-Fc (transmembrane activator & calcium modulator ligand interactor Fc-fusion) were used. This study layout relates to the data presented in FIGS. 1, 2, 4-10, and 15-17.

FIGS. 4A-4B show typical IgG levels and their complete suppression by SVP[Rapa] (FIG. 4B); BAFF inhibition seems to have an additional effect decreasing IgM response (FIG. 4A).

FIG. 5 shows IgG levels and their complete early suppression by SVP[Rapa] followed by 1/6 post-boost breakthrough. No breakthroughs in groups treated with aBAFF or TACI-Fc as of 18 days post-boost (shown by arrows).

FIGS. 6A-6D show IgM inhibition in [Rapa]− & [Rapa]+TACI-Fc-treated groups; more pronounced in [Rapa]+BAFF-treated mice.

FIG. 7 shows post-boost IgM dynamics in untreated group (post-boost elevation seen) and in SVP[Rapa]-treated group (high post-boost levels in a 1/6 breakthrough mouse); BAFF inhibition seems to have an additional effect decreasing IgM response; Fc-TACI does not add much to SVP[Rapa] at prime, but may give additional post-boost benefit.

FIG. 8 shows SEAP elevation by [Rapa]; further enhanced in presence of anti-BAFF.

FIGS. 9A-9D show consistent significant effects of a combo of [Rapa] and anti-BAFF for elevation of transgene (SEAP) expression.

FIG. 10 provides data from d21/28 pre-boost and then for up to 14 days after d37 boost. A combo of [Rapa] and anti-BAFF provides a consistent significant effect for elevation of transgene expression.

FIG. 11 shows the layout for another experiment. This study layout relates to the data presented in FIGS. 12-14 and 18-20.

FIGS. 12A-12B show early IgM and IgG dynamics for IgM suppression.

FIG. 13 demonstrates synergy with anti-BAFF and [Rapa] for IgM suppression.

FIG. 14 shows SEAP levels and the enhancement by [Rapa].

FIG. 15 shows AAV IgM levels in mice treated with AAV-SEAP alone, AAV-SEAP+SVP[RAPA], or AAV-SEAP+SVP[RAPA]+anti-BAFF at days 0, 37 and 155.

FIG. 16 shows AAV IgG levels in mice treated with AAV-SEAP alone, AAV-SEAP+SVP[RAPA], or AAV-SEAP+SVP[RAPA]+anti-BAFF at days 0, 37 and 155.

FIG. 17 shows SEAP levels in mice treated with AAV-SEAP alone, AAV-SEAP+SVP[RAPA], or AAV-SEAP+SVP[RAPA]+anti-BAFF at days 0, 37 and 155.

FIGS. 18A-18C show SEAP, IgM, and IgG levels in mice treated with AAV-SEAP alone, AAV-SEAP+SVP[RAPA], AAV-SEAP+anti-BAFF, or AAV-SEAP+SVP[RAPA]+anti-BAFF at days 0, 32 and 98. FIG. 18A shows SEAP levels. FIG. 18B shows IgM levels. FIG. 18C shows IgG levels.

FIGS. 19A-19F show SEAP, IgM, and IgG levels in mice treated with AAV-SEAP alone, AAV-SEAP+SVP[RAPA] (50 or 150 μg), or AAV-SEAP+SVP[RAPA] at days 0, 32, 98, and 160 with or without anti-BAFF either only on injection day or also given at 14 days after the 1st, the 3rd and the 4th AAV administrations. FIGS. 19A and 19B show SEAP levels at 50 μg (FIG. 19A) or 150 μg (FIG. 19B) rapamycin. FIGS. 19C and 19E show IgM levels. FIGS. 19D and 19F show IgG levels.

FIGS. 20A and 20B shows the correlation between SEAP and early d11 IgM levels in mice treated with AAV-SEAP+SVP[RAPA], or AAV-SEAP+SVP[RAPA]+anti-BAFF at days 0, 32, 98, and 160.

FIGS. 21A-21F show the proportion of different B cell populations in mice treated either with AAV-SEAP alone, AAV-SEAP+SVP[RAPA], AAV-SEAP+anti-BAFF, or AAV-SEAP+SVP[RAPA]+anti-BAFF (B, D, F), or the treatments w/o AAV, i.e., SVP[RAPA], anti-BAFF, or SVP[RAPA]+anti-BAFF (A, C, E).

FIGS. 22A-22F show IgM levels in mice treated with AAV-SEAP alone, AAV-SEAP+SVP[RAPA], or AAV-SEAP+SVP[RAPA]+ibritinub.

FIGS. 23A-23B show SEAP and its correlation with IgM levels in mice treated with AAV-SEAP alone, AAV-SEAP+SVP[RAPA], or AAV-SEAP+SVP[RAPA]+ibritinub. SEAP levels are shown in FIG. 23A Correlation of early day 6 IgM levels and late (d104/111) SEAP levels are shown in FIG. 23B.

FIGS. 24A-24B show IgM and IgG levels in mice treated with AAV-SEAP alone, AAV-SEAP+SVP[RAPA], AAV-SEAP+ibritinub, or AAV-SEAP+SVP[RAPA]+ibritinub. IgM levels are shown in FIG. 24A. IgG levels are shown in FIG. 24B.

FIG. 25 shows SEAP levels in mice treated with AAV-SEAP alone, AAV-SEAP+SVP[RAPA], AAV-SEAP+ibritinub, or AAV-SEAP+SVP[RAPA]+ibritinub.

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes. Such incorporation by reference is not intended to be an admission that any of the incorporated publications, patents and patent applications cited herein constitute prior art.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to “a DNA molecule” includes a mixture of two or more such DNA molecules or a plurality of such DNA molecules, reference to “an immunosuppressant” includes a mixture of two or more such immunosuppressant molecules or a plurality of such immunosuppressant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### A. Introduction

Viral transfer vectors are promising therapeutics for a variety of applications such as gene therapy, gene editing, gene expression modulation and exon skipping. Viral transfer vectors, therefore, may comprise transgenes that encode therapeutic proteins or nucleic acids. Unfortunately, the promise of these therapeutics has not yet been fully realized in a large part due to immune responses against the viral transfer vector. These immune responses include antibody, B cell and T cell responses and can be specific to viral antigens of the viral transfer vector, such as viral capsid or coat proteins or peptides thereof.

Surprisingly, it has been found that AAV induces an extremely strong and fast antibody production of both IgM and IgG, of which the latter is significantly blocked and the former delayed by synthetic nanocarriers comprising rapamycin. Also, surprisingly, treatment with a viral transfer vector in combination with synthetic nanocarriers comprising an immunosuppressant and an agent that suppresses the IgM response, e.g., an anti-IgM agent, such as an anti-BAFF monoclonal antibody, can have a synergistic effect on immune responses, such as IgM responses, and also results in a substantial increase in transgene expression after the first administration of a viral transfer vector.

Methods and compositions are provided that offer solutions to obstacles to effective use of viral transfer vectors for treatment. In particular, it has been unexpectedly discovered that IgM anti-viral transfer vector immune responses alone or in combination with other immune responses can be attenuated with the methods and related compositions provided herein. The methods and compositions can increase the efficacy of treatment with viral transfer vectors and provide for immune attenuation, even if the administration of the viral transfer vector need be repeated.

The invention will now be described in more detail below.

### B. Definitions

“Administering” or “administration” or “administer” means giving or dispensing a material to a subject in a manner that is pharmacologically useful. The term is intended to include “causing to be administered”. “Causing to be administered” means causing, urging, encouraging, aiding, inducing or directing, directly or indirectly, another party to administer the material. Any one of the methods provided herein may comprise or further comprise a step of administering concomitantly a viral transfer vector, synthetic nanocarriers comprising an immunosuppressant and an anti-IgM agent. In some embodiments, the concomitant administration is performed repeatedly. In still further embodiments, the concomitant administration is simultaneous administration.

“Amount effective” in the context of a composition or dosage form for administration to a subject as provided herein refers to an amount of the composition or dosage form that produces one or more desired results in the subject, for example, the reduction or elimination of an immune response, such as an IgM response, against a viral transfer vector or the generation of an anti-viral transfer vector attenuated response. The amount effective can be for in vitro or in vivo purposes. For in vivo purposes, the amount can be one that a clinician would believe may have a clinical benefit for a subject that may experience undesired immune responses as a result of administration of a viral transfer vector. In any one of the methods provided herein, the composition(s) administered may be in any one of the amounts effective as provided herein.

Amounts effective can involve reducing the level of an undesired immune response, although in some embodiments, it involves preventing an undesired immune response altogether. Amounts effective can also involve delaying the occurrence of an undesired immune response. An amount effective can also be an amount that results in a desired therapeutic endpoint or a desired therapeutic result. Amounts effective, preferably, result in a tolerogenic immune response in a subject to an antigen, such as a viral transfer vector antigen. Amounts effective, can also preferably result in increased transgene expression (a transgene being delivered by the viral transfer vector). This can be determined by measuring transgene expression in various tissues or systems of interest in the subject. This increased expression may be measured locally or systemically. The achievement of any of the foregoing can be monitored by routine methods.

In some embodiments of any one of the compositions and methods provided, the amount effective is one in which the desired immune response, such as the reduction or elimination of an immune response against a viral transfer vector or the generation of an anti-viral transfer vector attenuated response, persists in the subject for at least 1 week, at least 2 weeks or at least 1 month. In other embodiments of any one of the compositions and methods provided, the amount effective is one which produces a measurable desired immune response, such as the reduction or elimination of an immune response against a viral transfer vector or the generation of an anti-viral transfer vector attenuated response. In some embodiments, the amount effective is one that produces a measurable desired immune response (e.g., to a specific viral transfer vector antigen), for at least 1 week, at least 2 weeks or at least 1 month.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation.

“Anti-BAFF agent” refers to any agent, small molecules, antibodies, peptides, or nucleic acids, that is known to reduce the production, or levels of, or activity of BAFF. In some embodiments, an anti-BAFF agent is an anti-BAFF antibody. Exemplary anti-BAFF agents include, but are not limited to, TACI-Ig and soluble BAFF receptor.

“Anti-BAFF antibody” refers to any antibody that specifically binds to a BAFF polypeptide. For example, the anti-BAFF antibody may be a monoclonal antibody, such as Belimumab (Benlysta). In some instances, the anti-BAFF antibody can suppress the bioactivity of BAFF. Alternatively, or in addition, an anti-BAFF antibody may block the interaction between BAFF and its receptors, such as BAFF-R and BCMA (B cell maturation antigen). In some embodiments, a full intact antibody is used. In some embodiments, an antigen-binding fragment of the anti-BAFF antibody is instead used.

“Anti-IgM agent” refers to any agent, including but not limited to, small molecules, antibodies, peptides, or nucleic acids, that is known to reduce the production, or levels of, IgM, e.g., IgM antibodies. It will be appreciated by those of skill in the art that B cells generate antibodies. Thus, in some embodiments, an anti-IgM agent is any agent that is known to modulate or suppress B cell levels. In some embodiments, an anti-IgM agent is any agent that is known to modulate or suppress B cell maturation. In some embodiments, an anti-IgM agent is any agent that is known to modulate or suppress B cell activation. In some embodiments, an anti-IgM agent is any agent that is known to modulate or suppress T cell independent B cell activation.

Anti-IgM agents include, but are not limited to, IgM antagonist antibodies or antigen-binding fragments thereof that specifically bind to CD10, CD19, CD20, CD22, CD27, CD34, CD40, CD79a, CD79b, CD123, CD179b, FLT-3, ROR1, BR3, BAFF, or B7RP-1; IL21 modulating agents, e.g., IL-21 and IL-21 receptor antagonists; tyrosine kinase inhibitors, e.g., Syk inhibitors, BTK inhibitors, SRC protein tyrosine kinase inhibitors; PI3K inhibitors; PKC inhibitors; APRIL antagonists, e.g., TACI-Ig; mizoribine; tofacitinib; and tetracyclines.

“IgM antagonist antibodies” include, but are not limited to, antibodies that are known to reduce the production, or levels of, IgM, e.g., IgM antibodies. In some embodiments, an IgM antagonist antibody binds to and inhibits the activity of a protein or peptide involved in the production of, IgM, e.g., IgM antibodies, or in the modulation or stimulation immune pathway that leads to the production of, IgM, e.g., IgM antibodies.

In some embodiments, an IgM antagonist antibody is any antibody that is known to modulate B cell levels. In some embodiments, an IgM antagonist antibody is any antibody that is known to modulate B cell maturation. In some embodiments, an IgM antagonist antibody is any antibody that is known to modulate B cell activation. In some embodiments, an IgM antagonist antibody is any antibody that is known to modulate or suppress T cell independent B cell activation.

In some embodiments of any one of the methods, compositions or kits provided herein, an antigen-binding fragment of the antibody can be used in place of the antibody.

IgM antagonist antibodies or antigen-binding fragments thereof that specifically bind to CD10, CD19, CD20, CD22, CD27, CD34, CD40, CD79a, CD79b, CD123, CD179b, FLT-3, ROR1, BR3, BAFF, or B7RP-1 are examples of anti-IgM agents that can be used in any one of the methods, compositions or kits provided herein. Thus, such agents can also be antibodies or antigen-binding agents to B cell markers or other molecules that specifically bind such markers.

“APRIL antagonists” include, but are not limited to, any molecule that reduces or inhibits the function or the production of APRIL. A proliferation-inducing ligand (APRIL), also known as tumor necrosis factor ligand superfamily member 13 (TNFSF13), is a protein of the TNF superfamily recognized by the cell surface receptor TACI. APRIL is a ligand for TNFRSF17/BCMA, a member of the TNF receptor family. This protein and its receptor are both found to be important for B cell development. APRIL antagonists include small molecule inhibitors of APRIL, antibodies to APRIL, and antisense oligomers and RNAi inhibitors that reduce the expression of APRIL. Exemplary APRIL inhibitors include, but are not limited to, BION-1301 (Aduro Biotech, Inc.). In some embodiments, an APRIL antagonist is TACI-Ig. TACI-Ig is a recombinant fusion protein that combines the binding sites of BLyS and APRIL with the constant region of immunoglobin.

“Bruton's tyrosine kinase (BTK) inhibitors” include, but are not limited to, any molecule that reduces or inhibits the function or the production of a member of the BTK family of tyrosine kinases. A BTK inhibitor functions by inhibiting the tyrosine-protein kinase BTK enzyme, which plays an important role in B-cell development. BTK inhibitors include small molecule inhibitors of BTK, antibodies to BTK, and antisense oligomers and RNAi inhibitors that reduce the expression of BTK. Exemplary BTK inhibitors include, but are not limited to, AVL-292, CC-292, ONO-4059, ACP-196, PCI-32765, Acalabrutinib, GS-4059, spebrutinib, BGB-3111, and HM71224.

“IL-21 modulating agents” include, but are not limited to, any molecule that reduces or inhibits the function or the production of IL-21 or the IL-21 receptor. Interleukin-21 is a cytokine that has potent regulatory effects on cells of the immune system, including natural killer (NK) cells and cytotoxic T cells that can destroy virally infected or cancerous cells. IL-21 has been reported to contribute to the mechanism by which CD4+ T helper cells orchestrate the immune system response to viral infections. In some embodiments, an IL21 modulating agent is an IL-21 antagonist. IL-21 antagonists include small molecule inhibitors of IL-21, antibodies to IL-21, and antisense oligomers and RNAi inhibitors that reduce the expression of IL-21. Exemplary IL-21 inhibitors include, but are not limited to, NNC0114 (NovoNordisk). In some embodiments, and IL-21 modulating agent is an IL-21 receptor antagonist. IL-21 receptor antagonists include small molecule inhibitors of the IL-21 receptor, antibodies to the IL-21 receptor, and antisense oligomers and RNAi inhibitors that reduce the expression of the IL-21 receptor. Exemplary IL-21 receptor inhibitors include, but are not limited to, ATR-107(Pfizer).

“PI3K inhibitors” include, but are not limited to, any molecule that reduces or inhibits the function or the production of a member of the PI3K kinase family. PI3 kinases include, but are not limited to, PIK3CA, PIK3CB, PIK3CG, PIK3CD, PIK3R1, PIK3R2, PIK3R3, PIK3R4, PIK3R5, PIK3R6, PIK3C2A, PIK3C2B, PIK3C2G, and PIK3C3. PI3K inhibitors include small molecule inhibitors of PI3K, antibodies to PI3K, and antisense oligomers and RNAi inhibitors that reduce the expression of PI3K. Exemplary PI3K inhibitors include, but are not limited to, GS-1101, idelalisib, duvelisib, TGR-1202, AMG-319, copanlisib, wortmannin, LY294002, IC486068 and IC87114 (ICOS Corporation), and GDC-0941.

“PKC inhibitors” include, but are not limited to, any molecule that reduces or inhibits the function or the production of a member of the PKC kinase family. Protein Kinase C is a family of protein kinase enzymes that are involved in controlling the function of other proteins through the phosphorylation of hydroxyl groups of serine and threonine amino acid residues on these proteins, or a member of this family. PKC enzymes include, but are not limited to, PKC-α (PRKCA), PKC-β1 (PRKCB), PKC-β2 (PRKCB), PKC-γ (PRKCG), PKC-δ (PRKCD), PKC-ε (PRKCE), PKC-η (PRKCH), PKC-θ (PRKCQ), and PKC-ι (PRKCI), PKC-ζ (PRKCZ). PKC inhibitors include small molecule inhibitors of PKC, antibodies to PKC, and antisense oligomers and RNAi inhibitors that reduce the expression of PKC. Exemplary PKC inhibitors include, but are not limited to, enzastaurin, ruboxistaurin, chelerythrine, miyabenol C, myricitrin, gossypol, verbascoside, BIM-1, and bryostatin 1.

“SRC protein tyrosine kinase inhibitors” include, but are not limited to, any molecule that reduces or inhibits the function or the production of a member of the SRC kinase family. SRC inhibitors include small molecule inhibitors of SRC, antibodies to SRC, and antisense oligomers and RNAi inhibitors that reduce the expression of SRC. Exemplary Syk inhibitors include, but are not limited to, dasatinib.

“Syk inhibitors” include, but are not limited to, any molecule that reduces or inhibits the function or the production of a member of the Syk family of tyrosine kinases. Syk is involved in the transmission of signals from the B cell receptor and the T cell receptor. Syk inhibitors include small molecule inhibitors of Syk, antibodies to Syk, and antisense oligomers and RNAi inhibitors that reduce the expression of Syk. Exemplary Syk inhibitors include, but are not limited to, fostamatinib (R788), entospletinib (GS-9973), cerdulatinib (PRT062070), and TAK-659, entospletinib, and nilvadipine.

“Tetracyclines” are a group of broad-spectrum antibiotic compounds that have a common basic structure and can be isolated directly from several species of Streptomyces bacteria or produced at least semi-synthetically. Exemplary tetracyclines include, but are not limited to, chlortetracycline, oxytetracycline, demethylchlortetracycline, rolitetracycline, limecycline, clomocycline, methacycline, doxycycline, minocycline, and tertiary-butylglycylamidominocycline.

“Tyrosine kinase inhibitors” include, but are not limited to, any molecule that reduces or inhibits the function or the production of one or more tyrosine kinases. Tyrosine kinase inhibitors include small molecule inhibitors of tyrosine kinases, antibodies to tyrosine kinases, and antisense oligomers and RNAi inhibitors that reduce the expression of tyrosine kinases. Exemplary tyrosine kinase inhibitors include Syk inhibitors, BTK inhibitors, and SRC protein tyrosine kinase inhibitors. “Anti-viral transfer vector immune response” or “immune response against a viral transfer vector” or the like refers to any undesired immune response, such as an IgM response, against a viral transfer vector. In some embodiments, the undesired immune response is an antigen-specific immune response against the viral transfer vector or an antigen thereof. In some embodiments, the immune response is specific to a viral antigen of the viral transfer vector.

An anti-viral transfer vector immune response is said to be an “anti-viral transfer vector attenuated response” when it is in some manner reduced or eliminated in the subject or as compared to an expected or measured response in the subject or another subject. In some embodiments, the anti-viral transfer vector attenuated response in a subject comprises a reduced anti-viral transfer vector immune response (such as an IgM antibody response) measured using a biological sample obtained from the subject following a concomitant administration as provided herein as compared to an anti-viral transfer vector immune response measured using a biological sample obtained from another subject, such as a test subject, following administration to this other subject of the viral transfer vector without concomitant administration of the synthetic nanocarriers comprising an immunosuppressant and an anti-IgM agent. In some embodiments, the anti-viral transfer vector attenuated response is a reduced anti-viral transfer vector immune response (such as an IgM antibody response) in a biological sample obtained from the subject following a concomitant administration as provided herein upon a subsequent viral transfer vector in vitro challenge performed on the subject's biological sample as compared to the anti-viral transfer vector immune response detected upon viral transfer vector in vitro challenge performed on a biological sample obtained from another subject, such as a test subject, following administration to this other subject of the viral transfer vector without concomitant administration of synthetic nanocarriers comprising immunosuppressant and an anti-IgM agent.

“Antigen” means a B cell antigen or T cell antigen. “Type(s) of antigens” means molecules that share the same, or substantially the same, antigenic characteristics. In some embodiments, antigens may be proteins, polypeptides, peptides, lipoproteins, glycolipids, polynucleotides, polysaccharides, etc.

“Attach” or “Attached” or “Couple” or “Coupled” (and the like) means to chemically associate one entity (for example a moiety) with another. In some embodiments, the attaching is covalent, meaning that the attachment occurs in the context of the presence of a covalent bond between the two entities. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of attaching.

“Average”, as used herein, refers to the arithmetic mean unless otherwise noted.

“Concomitantly” means administering two or more materials/agents to a subject in a manner that is correlated in time, preferably sufficiently correlated in time so as to provide a modulation in an immune response, and even more preferably the two or more materials/agents are administered in combination. In embodiments, concomitant administration may encompass administration of two or more materials/agents within a specified period of time, preferably within 1 month, more preferably within 1 week, still more preferably within 1 day, and even more preferably within 1 hour. In embodiments, the materials/agents may be repeatedly administered concomitantly; that is concomitant administration on more than one occasion.

“Dosage form” means a pharmacologically and/or immunologically active material in a medium, carrier, vehicle, or device suitable for administration to a subject. Any one of the compositions or doses provided herein may be in a dosage form.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier.

“Escalating transgene expression” refers to increasing the level of a transgene expression product of a viral transfer vector in a subject, the transgene being delivered by the viral transfer vector. In some embodiments, the level of the transgene expression product may be determined by measuring transgene expression in various tissues or systems of interest in the subject. In some embodiments, the transgene expression product is a protein. In other embodiments, the transgene expression product is a nucleic acid. Escalating transgene expression can be determined, for example, by measuring the amount of the transgene expression product in a sample obtained from a subject and comparing it to a prior sample. The sample may be a tissue sample. In some embodiments, the transgene expression product can be measured using flow cytometry.

“Exon skipping transgene” means any nucleic acid that encodes an antisense oligonucleotide or other agent that can generate exon skipping. “Exon skipping” refers to an exon that is skipped and removed at the pre-mRNA level during protein production. Antisense oligonucleotides may interfere with splice sites or regulatory elements within an exon. This can lead to truncated, partially functional, protein despite the presence of a genetic mutation. Generally, the antisense oligonucleotides may be mutation-specific and bind to a mutation site in the pre-messenger RNA to induce exon skipping.

The subject may be one that has a disease or disorder in which exon skipping would be a benefit. The subject may have any one of the diseases or disorders provided herein in which generating exon skipping would be a benefit, such as a dystrophy. In addition, the exon skipping transgene may encode an agent that can generate exon skipping during the expression of any endogenous protein for which the result of exon skipping would confer a benefit. Examples of such proteins are the proteins associated with the diseases or disorders provided herein, such as any of the dystrophies provided herein. The proteins may also be the endogenous version of any one of the therapeutic proteins provided herein, in some embodiments.

“Gene editing transgene” means any nucleic acid that encodes an agent or component that is involved in a gene editing process. “Gene editing” generally refers to long-lasting or permanent modifications to genomic DNA, such as targeted DNA insertion, replacement, mutagenesis or removal. Gene editing may target DNA sequences that encode part or all of an expressed protein or target non-coding sequences of DNA that affect expression of a target gene(s). Gene editing may include the delivery of nucleic acids encoding a DNA sequence of interest and inserting the sequence of interest at a targeted site in genomic DNA using endonucleases. The endonucleases can create breaks in double-stranded DNA at desired locations in the genome and use the host cell's mechanisms to repair the break using homologous recombination, nonhomologous end-joining, etc. Classes of endonucleases that can be used for gene editing include, but are not limited to, meganucleases, zinc-finger nucleases (ZFNs), transcription activator-like effector nucleases (TALENs), clustered regularly interspaced short palindromic repeat(s) (CRISPR) and homing endonucleases.

The subject as provided herein may be one with any one of the diseases or disorders as provided herein, and the transgene is one that encodes a gene editing agent that may be used to correct a defect in any one of the proteins as provided herein, or an endogenous version thereof. Alternatively, in some embodiments a gene editing viral transfer vector may also include a transgene that encodes a therapeutic protein or portion thereof or nucleic acid as provided herein. In some embodiments, a gene editing viral transfer vector may be administered to a subject along with a viral transfer vector with a transgene that encodes a therapeutic protein or portion thereof or nucleic acid provided herein.

“Gene expression modulating transgene” refers to any nucleic acid that encodes a gene expression modulator. “Gene expression modulator” refers to a molecule that can enhance, inhibit or modulate the expression of one or more endogenous genes. Gene expression modulators, therefore, include DNA-binding proteins (e.g., artificial transcription factors) as well as molecules that mediate RNA interference. Gene expression modulators include RNAi molecules (e.g., dsRNAs or ssRNAs), miRNA, and triplex-forming oligonucleotides (TFOs). Gene expression modulators also may include modified RNAs, including modified versions of any of the foregoing RNA molecules.

The subject as provided herein may be one with any one of the diseases or disorders as provided herein, and the transgene is one that encodes a gene expression modulator that may be used to control expression of any one of the proteins provided herein. In some embodiments, the subject has a disease or disorder whereby the subject's endogenous version of the protein is defective or produced in limited amounts or not at all, and the gene expression modulator can control expression of such a protein. Thus, the gene expression modulator can, in some embodiments, control the expression of any one of the proteins as provided herein, or an endogenous version thereof (such as an endogenous version of a therapeutic protein as provided herein).

“Gene therapy transgene” refers to a nucleic acid that encodes an expression product such as a protein or nucleic acid and that when introduced into a cell can direct the expression of the protein or nucleic acid. When a protein, the protein can be a therapeutic protein. In some embodiments of any one of the methods or compositions provided herein, the subject to which the gene therapy transgene is administered by way of a viral transfer vector has a disease or disorder whereby the subject's endogenous version of the protein is defective or produced in limited amounts or not at all. In some embodiments, the encoded protein has no human counterpart but is predicted to provide therapeutically beneficial effects in the treatment of a disease or disorder.

“Immunosuppressant” means a compound that causes a tolerogenic effect, preferably through its effects on APCs. A tolerogenic effect generally refers to the modulation by the APC or other immune cells systemically and/or locally, that reduces, inhibits or prevents an undesired immune response to an antigen in a durable fashion. In one embodiment, the immunosuppressant is one that causes an APC to promote a regulatory phenotype in one or more immune effector cells. For example, the regulatory phenotype may be characterized by the inhibition of the production, induction, stimulation or recruitment of antigen-specific CD4+ T cells or B cells, the inhibition of the production of antigen-specific antibodies, the production, induction, stimulation or recruitment of Treg cells (e.g., CD4+CD25highFoxP3+ Treg cells), etc. This may be the result of the conversion of CD4+ T cells or B cells to a regulatory phenotype. This may also be the result of induction of FoxP3 in other immune cells, such as CD8+ T cells, macrophages and iNKT cells. In one embodiment, the immunosuppressant is one that affects the response of the APC after it processes an antigen. In another embodiment, the immunosuppressant is not one that interferes with the processing of the antigen. In a further embodiment, the immunosuppressant is not an apoptotic-signaling molecule. In another embodiment, the immunosuppressant is not a phospholipid.

In some embodiments, the immunosuppressant is an element that is in addition to the material that makes up the structure of the synthetic nanocarrier. For example, in one embodiment, where the synthetic nanocarrier is made up of one or more polymers, the immunosuppressant is a compound that is in addition and, in some embodiments, attached to the one or more polymers. As another example, in one embodiment, where the synthetic nanocarrier is made up of one or more lipids, the immunosuppressant is again in addition to and, in some embodiments, attached to the one or more lipids. In other embodiments, when the material of the synthetic nanocarrier also results in a tolerogenic effect, the immunosuppressant is an element present in addition to the material of the synthetic nanocarrier that results in a tolerogenic effect.

Immunosuppressants include, but are not limited to, statins; mTOR inhibitors, such as rapamycin or a rapamycin analog (i.e., rapalog); TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, retinoic acid, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide. Other exemplary immunosuppressants include, but are not limited, small molecule drugs, natural products, antibodies (e.g., antibodies against CD20, CD3, CD4), biologics-based drugs, carbohydrate-based drugs, RNAi, antisense nucleic acids, aptamers, methotrexate, NSAIDs; fingolimod; natalizumab; alemtuzumab; anti-CD3; tacrolimus (FK506), abatacept, belatacept, etc. “Rapalog” refers to a molecule that is structurally related to (an analog) of rapamycin (sirolimus). Examples of rapalogs include, without limitation, temsirolimus (CCI-779), everolimus (RAD001), ridaforolimus (AP-23573), and zotarolimus (ABT-578). Additional examples of rapalogs may be found, for example, in WO Publication WO 1998/002441 and U.S. Pat. No. 8,455,510, the rapalogs of which are incorporated herein by reference in their entirety.

Further immunosuppressants, are known to those of skill in the art, and the invention is not limited in this respect. In embodiments, the immunosuppressant may comprise any one of the agents provided herein.

“Load”, when coupled to a synthetic nanocarrier, is the amount of the immunosuppressant coupled to the synthetic nanocarrier based on the total dry recipe weight of materials in an entire synthetic nanocarrier (weight/weight). Generally, such a load is calculated as an average across a population of synthetic nanocarriers. In one embodiment, the load on average across the synthetic nanocarriers is between 0.1% and 50%. In another embodiment, the load is between 0.1% and 20%. In a further embodiment, the load is between 0.1% and 10%. In still a further embodiment, the load is between 1% and 10%. In still a further embodiment, the load is between 7% and 20%. In yet another embodiment, the load is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least at least 7%, at least 8%, at least 9%, at least 10%, at least 11%, at least 12%, at least 13%, at least 14%, at least 15%, at least 16%, at least 17%, at least 18%, at least 19%, at least 20% or at least 25% on average across the population of synthetic nanocarriers. In yet a further embodiment, the load is 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20% on average across the population of synthetic nanocarriers. In an embodiment of any one of the above embodiments, the load is no more than 25% on average across a population of synthetic nanocarriers. In embodiments, the load is calculated using any method known in the art. The load of an immunosuppressant comprise in synthetic nanocarriers may be any one of the loads provided herein.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., effective diameter) may be obtained, in some embodiments, by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicies of the sample. The effective diameter, or mean of the distribution, is then reported. Determining the effective sizes of high aspect ratio, or non-spheroidal, synthetic nanocarriers may require augmentative techniques, such as electron microscopy, to obtain more accurate measurements. “Dimension” or “size” or “diameter” of synthetic nanocarriers means the mean of a particle size distribution, for example, obtained using dynamic light scattering.

“Non-methoxy-terminated polymer” means a polymer that has at least one terminus that ends with a moiety other than methoxy. In some embodiments, the polymer has at least two termini that ends with a moiety other than methoxy. In other embodiments, the polymer has no termini that ends with methoxy. “Non-methoxy-terminated, pluronic polymer” means a polymer other than a linear pluronic polymer with methoxy at both termini. Polymeric nanoparticles as provided herein can comprise non-methoxy-terminated polymers or non-methoxy-terminated, pluronic polymers, in some embodiments. In other embodiments, polymeric nanoparticles do not comprise such polymers.

“Pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Protocol” means a pattern of administering to a subject and includes any dosing regimen of one or more substances to a subject. Protocols are made up of elements (or variables); thus a protocol comprises one or more elements. Such elements of the protocol can comprise dosing amounts (doses), dosing frequency, routes of administration, dosing duration, dosing rates, interval between dosing, combinations of any of the foregoing, and the like. In some embodiments, a protocol may be used to administer one or more compositions of the invention to one or more test subjects. Immune responses in these test subjects can then be assessed to determine whether or not the protocol was effective in generating a desired or desired level of an immune response or therapeutic effect. Any therapeutic and/or immunologic effect may be assessed. One or more of the elements of a protocol may have been previously demonstrated in test subjects, such as non-human subjects, and then translated into human protocols. For example, dosing amounts demonstrated in non-human subjects can be scaled as an element of a human protocol using established techniques such as alimetric scaling or other scaling methods. Whether or not a protocol had a desired effect can be determined using any of the methods provided herein or otherwise known in the art. For example, a sample may be obtained from a subject to which a composition provided herein has been administered according to a specific protocol in order to determine whether or not specific immune cells, cytokines, antibodies, etc. were reduced, generated, activated, etc. An exemplary protocol is one previously demonstrated to result in a tolerogenic immune response against a viral transfer vector antigen or to achieve any one of the beneficial results described herein. Useful methods for detecting the presence and/or number of immune cells include, but are not limited to, flow cytometric methods (e.g., FACS), ELISpot, proliferation responses, cytokine production, and immunohistochemistry methods. Antibodies and other binding agents for specific staining of immune cell markers, are commercially available. Such kits typically include staining reagents for antigens that allow for FACS-based detection, separation and/or quantitation of a desired cell population from a heterogeneous population of cells. In embodiments, a composition as provided herein is administered to a subject using one or more or all or substantially all of the elements of which a protocol is comprised, provided the selected element(s) are expected to achieve the desired result in the subject. Such expectation may be based on protocols determined in test subjects and scaling if needed. Any one of the methods provided herein may comprise or further comprise a step of administering a dose of a viral transfer vector in combination with synthetic nanocarriers comprising an immunosuppressant and an anti-IgM agent as described herein according to a protocol that has been shown to attenuate an anti-viral transfer vector immune response, such as an IgM response, and/or allow for the repeated administration of a viral transfer vector and/or result in the attenuation of one or more other immune responses against the viral transfer vector and/or result in increased transgene expression. Any one of the methods provided herein may comprise or further comprise determining such a protocol that achieves any one or more of the beneficial results described herein. Any one of the methods provided herein may comprise or further comprise a step of administering according to a protocol that achieves any one or more of the beneficial results described herein.

“Repeat dose” or “repeat dosing” or the like means at least one additional dose or dosing that is administered to a subject subsequent to an earlier dose or dosing of the same material. For example, a repeated dose of a viral transfer vector is at least one additional dose of the viral transfer vector after a prior dose of the same material. While the material may be the same, the amount of the material in the repeated dose may be different from the earlier dose. A repeat dose may be administered as provided herein, such as in the intervals of the Examples. Repeat dosing is considered to be efficacious if it results in a beneficial effect for the subject. Preferably, efficacious repeat dosing results in a beneficial effect, such as a therapeutic effect, in conjunction with an attenuated anti-viral transfer vector response.

“Simultaneous” means administration at the same time or substantially at the same time where a clinician would consider any time between administrations virtually nil or negligible as to the impact on the desired therapeutic outcome. In some embodiments, simultaneous means that the administrations occur with 5, 4, 3, 2, 1 or fewer minutes.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like. As used herein, a subject may be one in need of any one of the methods or compositions provided herein.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, synthetic nanocarriers do not comprise chitosan. In other embodiments, synthetic nanocarriers are not lipid-based nanoparticles. In further embodiments, synthetic nanocarriers do not comprise a phospholipid.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid attached virus-like particles disclosed in published US Patent Application 20060251677 to Bachmann et al., (9) the virus-like particles disclosed in WO2010047839A1 or WO2009106999A2, (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), (11) apoptotic cells, apoptotic bodies or the synthetic or semisynthetic mimics disclosed in U.S. Publication 2002/0086049, or (12) those of Look et al., Nanogel-based delivery of mycophenolic acid ameliorates systemic lupus erythematosus in mice” J. Clinical Investigation 123(4):1741-1749(2013).

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, synthetic nanocarriers may possess an aspect ratio greater than 1:1, 1:1.2, 1:1.5, 1:2, 1:3, 1:5, 1:7, or greater than 1:10.

“Therapeutic protein” means any protein that may be expressed from a gene therapy transgene as provided herein. The therapeutic protein may be one used for protein replacement or protein supplementation. Therapeutic proteins include, but are not limited to, enzymes, enzyme cofactors, hormones, blood clotting factors, cytokines, growth factors, etc. Examples of other therapeutic proteins are provided elsewhere herein. A subject may be one in need of treatment with any one of the therapeutic proteins provided herein.

“Transgene of the viral transfer vector” refers to the nucleic acid material the viral transfer vector is used to transport into a cell and, once in the cell, may be expressed to produce a protein or nucleic acid molecule, such as for a therapeutic application as described herein. The transgene may be a gene therapy transgene, a gene editing transgene, a gene expression modulating transgene or an exon skipping transgene. “Expressed” or “expression” or the like refers to the synthesis of a functional (i.e., physiologically active for the desired purpose) gene product after the transgene is transduced into a cell and processed by the transduced cell. Such a gene product is also referred to herein as a “transgene expression product”. The expressed products include, therefore, the resultant protein or nucleic acid, such as an antisense oligonucleotide or a therapeutic RNA, encoded by the transgene.

“Viral transfer vector” means a viral vector that has been adapted to deliver a nucleic acid, such as a transgene, as provided herein and includes such nucleic acid. “Viral vector” refers to all of the viral components of a viral transfer vector. Accordingly, “viral antigen” refers to an antigen of the viral components of the viral transfer vector, such as a capsid or coat protein, but not to the nucleic acid, such as a transgene, that it delivers, or any product it encodes. “Viral transfer vector antigen” refers to any antigen of the viral transfer vector including its viral components as well as delivered nucleic acid, such as a transgene, or any expression product thereof. The transgene may be a gene therapy transgene, a gene editing transgene, a gene expression modulating transgene or an exon skipping transgene. In some embodiments, the transgene is one that encodes a protein provided herein, such as a therapeutic protein, a DNA-binding protein or an endonuclease. In other embodiments, the transgene is one that encodes guide RNA, an antisense nucleic acid, snRNA, an RNAi molecule (e.g., dsRNAs or ssRNAs), miRNA, or triplex-forming oligonucleotides (TFOs), etc. Viral vectors can be based on, without limitation, retroviruses (e.g., murine retrovirus, avian retrovirus, Moloney murine leukemia virus (MoMuLV), Harvey murine sarcoma virus (HaMuSV), murine mammary tumor virus (MuMTV), gibbon ape leukemia virus (GaLV) and Rous Sarcoma Virus (RSV)), lentiviruses, herpes viruses, adenoviruses, adeno-associated viruses, alphaviruses, etc. Other examples are provided elsewhere herein or are known in the art. The viral vectors may be based on natural variants, strains, or serotypes of viruses, such as any one of those provided herein. The viral vectors may also be based on viruses selected through molecular evolution. The viral vectors may also be engineered vectors, recombinant vectors, mutant vectors, or hybrid vectors. In some embodiments, the viral vector is a “chimeric viral vector”. In such embodiments, this means that the viral vector is made up of viral components that are derived from more than one virus or viral vector.

### C. Compositions for Use in the Inventive Methods

Importantly, the methods and compositions provided herein have been found to attenuate immune responses, such as IgM responses, against viral transfer vectors. Additionally, the methods and compositions provided herein have been found to enable a substantial increase in transgene expression. The methods and compositions provided herein are useful for the treatment of subjects with a viral transfer vector. Viral transfer vectors can be used to deliver nucleic acids, such as transgenes, for a variety of purposes, including for gene therapy, gene editing, gene expression modulation and exon skipping, the methods and compositions provided herein are also so applicable.

### Transgenes

The transgene of the viral transfer vectors provided herein may be a gene therapy transgene and may encode any protein or portion thereof beneficial to a subject, such as one with a disease or disorder. The protein may be an extracellular, intracellular or membrane-bound protein. The protein can be a therapeutic protein, and the subject to which the gene therapy transgene is administered by way of a viral transfer vector can have a disease or disorder whereby the subject's endogenous version of the protein is defective or produced in limited amounts or not at all. Thus, the subject may be one with any one of the diseases or disorders as provided herein, and the transgene may be one that encodes any one of the therapeutic proteins or portion thereof as provided herein.

Examples of therapeutic proteins include, but are not limited to, infusible or injectable therapeutic proteins, enzymes, enzyme cofactors, hormones, blood or blood coagulation factors, cytokines and interferons, growth factors, adipokines, etc.

Examples of infusible or injectable therapeutic proteins include, for example, Tocilizumab (Roche/Actemra®), alpha-1 antitryp sin (Kamada/AAT), Hematide® (Affymax and Takeda, synthetic peptide), albinterferon alfa-2b (Novartis/Zalbin™), Rhucin® (Pharming Group, C1 inhibitor replacement therapy), tesamorelin (Theratechnologies/Egrifta, synthetic growth hormone-releasing factor), ocrelizumab (Genentech, Roche and Biogen), belimumab (GlaxoSmithKline/Benlysta®), pegloticase (Savient Pharmaceuticals/Krystexxa™), taliglucerase alfa (Protalix/Uplyso), agalsidase alfa (Shire/Replagal®), and velaglucerase alfa (Shire).

Examples of enzymes include lysozyme, oxidoreductases, transferases, hydrolases, lyases, isomerases, asparaginases, uricases, glycosidases, proteases, nucleases, collagenases, hyaluronidases, heparinases, heparanases, kinases, phosphatases, lysins and ligases. Other examples of enzymes include those that used for enzyme replacement therapy including, but not limited to, imiglucerase (e.g., CEREZYME™), a-galactosidase A (a-gal A) (e.g., agalsidase beta, FABRYZYME™), acid a-glucosidase (GAA) (e.g., alglucosidase alfa, LUMIZYME™, MYOZYME™), and arylsulfatase B (e.g., laronidase, ALDURAZYME™, idursulfase, ELAPRASE™, arylsulfatase B, NAGLAZYME™).

Examples of hormones include, but are not limited to, gonadotropins, thyroid-stimulating hormone, melanocortins, pituitary hormones, vasopressin, oxytocin, growth hormones, prolactin, orexins, natriuretic hormones, parathyroid hormone, calcitonins, erythropoietin, and pancreatic hormones.

Examples of blood or blood coagulation factors include Factor I (fibrinogen), Factor II (prothrombin), tissue factor, Factor V (proaccelerin, labile factor), Factor VII (stable factor, proconvertin), Factor VIII (antihemophilic globulin), Factor IX (Christmas factor or plasma thromboplastin component), Factor X (Stuart-Prower factor), Factor Xa, Factor XI, Factor XII (Hageman factor), Factor XIII (fibrin-stabilizing factor), von Willebrand factor, von Heldebrant Factor, prekallikrein (Fletcher factor), high-molecular weight kininogen (HMWK) (Fitzgerald factor), fibronectin, fibrin, thrombin, antithrombin, such as antithrombin III, heparin cofactor II, protein C, protein S, protein Z, protein Z-related protease inhibitot (ZPI), plasminogen, alpha 2-antiplasmin, tissue plasminogen activator (tPA), urokinase, plasminogen activator inhibitor-1 (PAI1), plasminogen activator inhibitor-2 (PAI2), cancer procoagulant, and epoetin alfa (Epogen, Procrit).

Examples of cytokines include lymphokines, interleukins, and chemokines, type 1 cytokines, such as IFN-γ, TGF-β, and type 2 cytokines, such as IL-4, IL-10, and IL-13.

Examples of growth factors include Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Transforming growth factor beta(TGF-β), Tumour necrosis factor-alpha(TNF-α), Vascular endothelial growth factor (VEGF), Wnt Signaling Pathway, placental growth factor (PlGF), [(Foetal Bovine Somatotrophin)] (FBS), IL-1, IL-2, IL-3, IL-4, IL-5, IL-6, and IL-7.

Examples of adipokines, include leptin and adiponectin.

Additional examples of therapeutic proteins include, but are not limited to, receptors, signaling proteins, cytoskeletal proteins, scaffold proteins, transcription factors, structural proteins, membrane proteins, cytosolic proteins, binding proteins, nuclear proteins, secreted proteins, golgi proteins, endoplasmic reticulum proteins, mitochondrial proteins, and vesicular proteins, etc.

The transgene of the gene therapy viral transfer vectors provided herein may encode a functional version of any protein that through some defect in the endogenous version of which in a subject (including a defect in the expression of the endogenous version) results in a disease or disorder in the subject. Examples of such diseases or disorders include, but are not limited to, lysosomal storage diseases/disorders, such as Santavuori-Haltia disease (Infantile Neuronal Ceroid Lipofuscinosis Type 1), Jansky-Bielschowsky Disease (late infantile neuronal ceroid lipofuscinosis, Type 2), Batten disease (juvenile neuronal ceroid lipofuscinosis, Type 3), Kufs disease (neuronal ceroid lipofuscinosis, Type 4), Von Gierke disease (glycogen storage disease, Type Ia), glycogen storage disease, Type Ib, Pompe disease (glycogen storage disease, Type II), Forbes or Cori disease (glycogen storage disease, Type III), mucolipidosis II (I-Cell disease), mucolipidosis III (Pseudo-Hurler polydystrophy), mucolipdosis IV (sialolipidosis), cystinosis (adult nonnephropathic type), cystinosis (infantile nephropathic type), cystinosis (juvenile or adolescent nephropathic), Salla disease/infantile sialic acid storage disorder, and saposin deficiencies; disorders of lipid and sphingolipid degradation, such as GM1 gangliosidosis (infantile, late infantile/juvenile, and adult/chronic), Tay-Sachs disease, Sandhoff disease, GM2 gangliodisosis, Ab variant, Fabry disease, Gaucher disease, Types I, II and III, metachromatic leukidystrophy, Krabbe disease (early and late onset), Neimann-Pick disease, Types A, B, C1, and C2, Farber disease, and Wolman disease (cholesteryl esther storage disease); disorders of mucopolysaccharide degradation, such as Hurler syndrome (MPSI), Scheie syndrome (MPS IS), Hurler-Scheie syndrome (MPS IH/S), Hunter syndrome (MPS II), Sanfillippo A syndrome (MPS IIIA), Sanfillippo B syndrome (MPS IIIB), Sanfillippo C syndrome (MPS IIIC), Sanfillippo D syndrome (MPS IIID), Morquio A syndrome (MPS IVA), Morquio B syndrome (MPS IVB), Maroteaux-Lamy syndrome (MPS VI), and Sly syndrome (MPS VII); disorders of glycoprotein degradation, such as alpha mannosidosis, beta mannosidosis, fucosidosis, asparylglucosaminuria, mucolipidosis I (sialidosis), galactosialidosis, Schindler disease, and Schindler disease, Type II/Kanzaki disease; and leukodystrophy diseases/disorders, such as abetalipoproteinemia, neonatal adrenoleukodystrophy, Canavan disease, cerebrotendinous xanthromatosis, Pelizaeus Merzbacher disease, Tangier disease, Refum disease, infantile, and Refum disease, classic.

Additional examples of such diseases/disorders of a subject as provided herein include, but are not limited to, acid maltase deficiency (e.g., Pompe disease, glycogenosis type 2, lysosomal storage disease); carnitine deficiency; carnitine palmityl transferase deficiency; debrancher enzyme deficiency (e.g., Cori or Forbes disease, glycogenosis type 3); lactate dehydrogenase deficiency (e.g., glycogenosis type 11); myoadenylate deaminase deficiency; phosphofructokinase deficiency (e.g., Tarui disease, glycogenosis type 7); phosphogylcerate kinase deficiency (e.g., glycogenosis type 9); phosphogylcerate mutase deficiency (e.g., glycogenosis type 10); phosphorylase deficiency (e.g., McArdle disease, myophosphorylase deficiency, glycogenosis type 5); Gaucher's Disease (e.g., chromosome 1, enzyme glucocerebrosidase affected); Achondroplasia (e.g., chromosome 4, fibroblast growth factor receptor 3 affected); Huntington's Disease (e.g., chromosome 4, huntingtin); Hemochromatosis (e.g., chromosome 6, HFE protein); Cystic Fibrosis (e.g., chromosome 7, CFTR); Friedreich's Ataxia (chromosome 9, frataxin); Best Disease (chromosome 11, VMD2); Sickle Cell Disease (chromosome 11, hemoglobin); Phenylketoniuria (chromosome 12, phenylalanine hydroxylase); Marfan's Syndrome (chromosome 15, fibrillin); Myotonic Dystophy (chromosome 19, dystophia myotonica protein kinase); Adrenoleukodystrophy (x-chromosome, lignoceroyl-CoA ligase in peroxisomes); Duchene's Muscular Dystrophy (x-chromosome, dystrophin); Rett Syndrome (x-chromosome, methylCpG-binding protein 2); Leber's Hereditary Optic Neuropathy (mitochondria, respiratory proteins); Mitochondria Encephalopathy, Lactic Acidosis and Stroke (MELAS) (mitochondria, transfer RNA); and Enzyme deficiencies of the Urea Cycle.

Still additional examples of such diseases or disorders include, but are not limited to, Sickle Cell Anemia, Myotubular Myopathy, Hemophilia B, Lipoprotein lipase deficiency, Ornithine Transcarbamylase Deficiency, Crigler-Najjar Syndrome, Mucolipidosis IV, Niemann-Pick A, Sanfilippo A, Sanfilippo B, Sanfilippo C, Sanfilippo D, b-thalassaemia and Duchenne Muscular Dystrophy. Still further examples of diseases or disorders include those that are the result of defects in lipid and sphingolipid degradation, mucopolysaccharide degradation, glycoprotein degradation, leukodystrophies, etc.

The functional versions of the defective proteins of any one of the disease or disorders provided herein may be encoded by the transgene of a gene therapy viral transfer vector and are also considered therapeutic proteins. It follows that therapeutic proteins also include Myophosphorylase, glucocerebrosidase, fibroblast growth factor receptor 3, huntingtin, HFE protein, CFTR, frataxin, VMD2, hemoglobin, phenylalanine hydroxylase, fibrillin, dystophia myotonica protein kinase, lignoceroyl-CoA ligase, dystrophin, methylCpG-binding protein 2, Beta hemoglobin, Myotubularin, Cathepsin A, Factor IX, Lipoprotein lipase, Beta galactosidase, Ornithine Transcarbamylase, Iduronate-2-Sulfatase, Acid-Alpha Glucosidase, UDP-glucuronosyltransferase 1-1, GlcNAc-1-phosphotransferase, GlcNAc-1-phosphotransferase, Mucolipin-1, Microsomal triglyceride transfer protein, Sphingomyelinase, Acid ceramidase, Lysosomal acid lipase, Alpha-L-iduronidase, Heparan N-sulfatase, alpha-N-acetylglucosaminidase, acetyl-CoA alpha-glucosaminide acetyltransferase, N-acetylglucosamine 6-sulfatase, N-acetylgalactosamine-6 sulfatase, Alpha-mannosidase, Alpha-galactosidase A, Cystic fibrosis conductance transmembrane regulator, and respiratory proteins.

As further examples, therapeutic proteins also include functional versions of proteins associated with disorders of lipid and sphingolipid degradation (e.g., β-Galactosidase-1, β-Hexosaminidase A, β-Hexosaminidases A and B, GM2 Activator Protein, 8-Galactosidase A, Glucocerebrosidase, Glucocerebrosidase, Glucocerebrosidase, Arylsulfatase A, Galactosylceramidase, Sphingomyelinase, Sphingomyelinase, NPC1, HE1 protein (Cholesterol Trafficking Defect), Acid Ceramidase, Lysosomal Acid Lipase); disorders of mucopolysaccharide degradation (e.g., L-Iduronidase, L-Iduronidase, L-Iduronidase, Iduronate Sulfatase, Heparan N-Sulfatase, N-Acetylglucosaminidase, Acetyl-CoA-Glucosaminidase, Acetyltransferase, Acetylglucosamine-6-Sulfatase, Galactosamine-6-Sulfatase, Arylsulfatase B, Glucuronidase); disorders of glycoprotein degradation (e.g., Mannosidase, mannosidase, 1-fucosidase, Aspartylglycosaminidase, Neuraminidase, Lysosomal protective protein, Lysosomal 8-N-acetylgalactosaminidase, Lysosomal 8-N-acetylgalactosaminidase); lysosomal storage disorders (e.g., Palmitoyl-protein thioesterase, at least 4 subtypes, Lysosomal membrane protein, Unknown, Glucose-6-phosphatase, Glucose-6-phosphate translocase, Acid maltase, Debrancher enzyme amylo-1,6 glucosidase, N-acetylglucosamine-1-phosphotransferase, N-acetylglucosamine-1-phosphotransferase, Ganglioside sialidase (neuraminidase), Lysosomal cystine transport protein, Lysosomal cystine transport protein, Lysosomal cystine transport protein, Sialic acid transport protein Saposins, A, B, C, D) and leukodystrophies (e.g., Microsomal triglyceride transfer protein/apolipoprotein B, Peroxisomal membrane transfer protein, Peroxins, Aspartoacylase, Sterol-27-hydroxlase, Proteolipid protein, ABC1 transporter, Peroxisome membrane protein 3 or Peroxisome biogenesis factor 1, Phytanic acid oxidase).

The viral transfer vectors provided herein may be used for gene editing. In such embodiments, the transgene of the viral transfer vector is a gene editing transgene. Such a transgene encodes an agent or component that is involved in a gene editing process. Generally, such a process results in long-lasting or permanent modifications to genomic DNA, such as targeted DNA insertion, replacement, mutagenesis or removal. Gene editing may include the delivery of nucleic acids encoding a DNA sequence of interest and inserting the sequence of interest at a targeted site in genomic DNA using endonucleases. Thus, gene editing transgenes may comprise these nucleic acids encoding a DNA sequence of interest for insertion. In some embodiments, the DNA sequence for insertion is a DNA sequence encoding any one of the therapeutic proteins provided herein. Alternatively, or in addition to, the gene editing transgene may comprise nucleic acids that encode one of more components that can alone or in combination with other components carry out the gene editing process. The gene editing transgenes provided herein may encode an endonuclease and/or a guide RNA, etc.

Endonucleases can create breaks in double-stranded DNA at desired locations in a genome and use the host cell's mechanisms to repair the break using homologous recombination, nonhomologous end-joining, etc. Classes of endonucleases that can be used for gene editing include, but are not limited to, meganucleases, zinc-finger nucleases (ZFNs), transcription activator-like effector nucleases (TALENs), clustered regularly interspaced short palindromic repeat(s) (CRISPR) and homing endonucleases. The gene editing transgene of the viral transfer vectors provided herein may encode any one of the endonucleases provided herein.

Meganucleases are generally characterized by their capacity to recognize and cut DNA sequences (˜14-40 base pairs). In addition, known techniques, such as mutagenesis and high-throughput screening and combinatorial assembly, can be used to create custom meganucleases, where protein subunits can be associated or fused. Examples of meganucleases can be found in U.S. Pat. Nos. 8,802,437, 8,445,251 and 8,338,157; and U.S. Publication Nos. 20130224863, 20110113509 and 20110033935, the meganucleases of which are incorporated herein by reference.

A zinc finger nuclease typically comprises a zinc finger domain that binds a specific target site within a nucleic acid molecule, and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Zinc finger nucleases can be designed to target virtually any desired sequence in a given nucleic acid molecule for cleavage. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich N P, Pabo C O (May 1991). “Zinc finger-DNA recognition: crystal structure of a Zif268-DNA complex at 2.1 A”. Science 252 (5007): 809-17, the entire contents of which are incorporated herein). In some embodiments, bacterial or phage display is employed to develop a zinc finger domain that recognizes a desired nucleic acid sequence, for example, a desired endonuclease target site. Zinc finger nucleases, in some embodiments, comprise a zinc finger binding domain and a cleavage domain fused or otherwise conjugated to each other via a linker, for example, a polypeptide linker. The length of the linker can determine the distance of the cut from the nucleic acid sequence bound by the zinc finger domain. Examples of zinc finger nucleases can be found in U.S. Pat. Nos. 8,956,828; 8,921,112; 8,846,578; 8,569,253, the zinc finger nucleases of which are incorporated herein by reference.

Transcription activator-like effector nucleases (TALENs) are artificial restriction enzymes produced by fusing specific DNA binding domains to generic DNA cleaving domains. The DNA binding domains, which can be designed to bind any desired DNA sequence, come from transcription activator-like (TAL) effectors, DNA-binding proteins excreted by certain bacteria that infect plants. Transcription activator-like effectors (TALEs) can be engineered to bind practically any DNA sequence or joined together into arrays in combination with a DNA cleavage domain. TALENs can be used similarly to design zinc finger nucleases. Examples of TALENS can be found in U.S. Pat. No. 8,697,853; as well as U.S. Publication Nos. 20150118216, 20150079064, and 20140087426, the TALENS of which are incorporated herein by reference.

The CRISPR (clustered regularly interspaced short palindromic repeats)/Cas system can also be used for gene editing. In a CRISPR/Cas system, guide RNA (gRNA) is encoded genomically or episomally (e.g., on a plasmid). The gRNA forms a complex with an endonuclease, such as Cas9 endonuclease, following transcription. The complex is then guided by the specificity determining sequence (SDS) of the gRNA to a DNA target sequence, typically located in the genome of a cell. Cas9 or Cas9 endonuclease refers to an RNA-guided endonuclease comprising a Cas9 protein, or a fragment thereof (e.g., a protein comprising an active or inactive DNA cleavage domain of Cas9 or a partially inactive DNA cleavage domain (e.g., a Cas9 nickase), and/or the gRNA binding domain of Cas9). Cas9 recognizes a short motif in the CRISPR repeat sequences (the PAM or protospacer adjacent motif) to help distinguish self versus non-self. Cas9 endonuclease sequences and structures are well known to those of skill in the art (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L. expand/collapse author list McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012)). Single guide RNAs (“sgRNA”, or simply “gNRA”) can be engineered so as to incorporate aspects of both the crRNA and tracrRNA into a single RNA species. See e.g., Jinek M., Chylinski K., Fonfara I., Hauer M., Doudna J. A., Charpentier E. Science 337:816-821(2012).

Cas9 orthologs have been described in various species, including, but not limited to, S. pyogenes and S. thermophilus. Additional suitable Cas9 endonucleases and sequences will be apparent to those of skill in the art, and such Cas9 endonucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737. In some embodiments, a gene editing transgene encodes a wild-type Cas9, fragment or a Cas9 variant. A “Cas9 variant” is any protein with a Cas9 function that is not identical to a Cas9 wild-type endonuclease as it occurs in nature. In some embodiments, a Cas9 variant shares homology to a wild-type Cas9, or a fragment thereof. A Cas9 variant in some embodiments has at least 40% sequence identity to Streptococcus pyogenes or S. thermophilus Cas9 protein and retains the Cas9 functionality. Preferably, the sequence identity is at least 90%, 95%, or more. More preferably, the sequence identity is at least 98% or 99% sequence identity. In some embodiments of any one of the Cas9 variants for use in any one of the methods provided herein the sequence identity is amino acid sequence identity. Cas9 variants also include Cas9 dimers, Cas9 fusion proteins, Cas9 fragments, minimized Cas9 proteins, Cas9 variants without a cleavage domain, Cas9 variants without a gRNA domain, Cas9-recombinase fusions, fCas9, FokI-dCas9, etc. Examples of such Cas9 variants can be found, for example, in U.S. Publication Nos. 20150071898 and 20150071899, the description of Cas9 proteins and Cas9 variants of which is incorporated herein by reference. Cas9 variants also include Cas9 nickases, which comprise mutation(s) which inactivate a single endonuclease domain in Cas9. Such nickases can induce a single strand break in a target nucleic acid as opposed to a double strand break. Cas9 variants also include Cas9 null nucleases, a Cas9 variant in which one nuclease domain is inactivated by a mutation. Examples of additional Cas9 variants and/or methods of identifying further Cas9 variants can be found in U.S. Publication Nos. 20140357523, 20150165054 and 20150166980, the contents of which pertaining to Cas9 proteins, Cas9 variants and methods of their identification being incorporated herein by reference.

Still other examples of Cas9 variants include a mutant form, known as Cas9D10A, with only nickase activity. Cas9D10A is appealing in terms of target specificity when loci are targeted by paired Cas9 complexes designed to generate adjacent DNA nicks. Another example of a Cas9 variant is a nuclease-deficient Cas9 (dCas9). Mutations H840A in the HNH domain and D10A in the RuvC domain inactivate cleavage activity, but do not prevent DNA binding. Therefore, this variant can be used to sequence-specifically target any region of the genome without cleavage. Instead, by fusing with various effector domains, dCas9 can be used either as a gene silencing or activation tool. The gene editing transgene, in some embodiments, may encode any one of the Cas9 variants provided herein.

Methods of using RNA-programmable endonucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013)).

Homing endonucleases can catalyze, at few or singular locations, the hydrolysis of the genomic DNA used to synthesize them, thereby transmitting their genes horizontally within a host, increasing their allele frequency. Homing endonucleases generally have long recognition sequences, they thereby have low probability of random cleavage. One allele carries the gene (homing endonuclease gene+, HEG+), prior to transmission, while the other does not (HEG−), and is susceptible to enzyme cleavage. The enzyme, once synthesized, breaks the chromosome in the HEG− allele, initiating a response from the cellular DNA repair system which takes the pattern of the opposite, using recombination, undamaged DNA allele, HEG+, that contains the gene for the endonuclease. Thus, the gene is copied to another allele that initially did not have it, and it is propagated through successively. Examples of homing endonucleases can be found, for example, in U.S. Publication No. 20150166969; and U.S. Pat. No. 9,005,973, the homing endonucleases of which are incorporated herein by reference.

The viral transfer vectors provided herein may be used for gene expression modulation. In such embodiments, the transgene of the viral transfer vector is a gene expression modulating transgene. Such a transgene encodes a gene expression modulator that can enhance, inhibit or modulate the expression of one or more endogenous genes. The endogenous gene may encode any one of the proteins as provided herein provided the protein is an endogenous protein of the subject. Accordingly, the subject may be one with any one of the diseases or disorders provided herein where there would be a benefit provided by gene expression modulation.

Gene expression modulators include DNA-binding proteins (e.g., artificial transcription factors, such as those of U.S. Publication No. 20140296129, the artificial transcription factors of which are incorporated herein by reference; and transcriptional silencer protein NRF of U.S. Publication No. 20030125286, the transcriptional silencer protein NRF of which is incorporated herein by reference) as well as therapeutic RNAs. Therapeutic RNAs include, but are not limited to, inhibitors of mRNA translation (antisense), agents of RNA interference (RNAi), catalytically active RNA molecules (ribozymes), transfer RNA (tRNA) and RNAs that bind proteins and other molecular ligands (aptamers). Gene expression modulators include any agents of the foregoing and include antisense nucleic acids, RNAi molecules (e.g., double-stranded RNAs (dsRNAs), single-stranded RNAs (ssRNAs), micro RNAs (miRNAs), short interfering RNAs (siRNAs), short hairpin RNAs (shRNAs)) and triplex-forming oligonucleotides (TFOs). Gene expression modulators also may include modified versions of any of the foregoing RNA molecules and, thus, include modified mRNAs, such as synthetic chemically modified RNAs.

The gene expression modulator may be an antisense nucleic acid. Antisense nucleic acids can provide for the targeted inhibition of gene expression (e.g., the expression of mutant protein, a dominantly active gene product, a protein associated with toxicity or gene products that are introduced into a cell by an infectious agent, such as a virus). Thus, gene expression modulating viral transfer vectors can be used for treating diseases or disorders associated with dominant-negative or gain-of-function pathogenetic mechanisms, cancer, or infection. The subject of any one of the methods provided herein may be a subject that has a viral infection, inflammatory disorder, cardiovascular disease, cancer, genetic disorder or autoimmune disease. Antisense nucleic acids may also interfere with mRNA splicing machinery and disrupt normal cellular mRNA processing. Accordingly, the gene expression modulating transgene may encode elements that interact with spliceosome proteins. Examples of antisense nucleic acids (and related constructs) can be found in, for example, U.S. Publication Nos. 20050020529 and 20050271733, the antisense nucleic acids and constructs of which are incorporated herein by reference.

The gene expression modulator may also be a ribozyme (i.e., a RNA molecule that can cleave other RNAs, such as single-stranded RNA). Such molecules may be engineered to recognize specific nucleotide sequences in a RNA molecule and cleave it (Cech, J. Amer. Med. Assn., 260:3030, 1988). For example, ribozymes can be engineered so that only mRNAs with sequences complementary to a construct containing the ribozyme are inactivated. Types of ribozymes and how to prepare related constructs are known in the art (Hasselhoff, et al., Nature, 334:585, 1988; and U.S. Publication No. 20050020529, the teachings of which pertaining to such ribozymes and methods are incorporated herein by reference).

The gene expression modulator may be an interfering RNA (RNAi). RNA interference refers to the process of sequence-specific post-transcriptional gene silencing mediated by interfering RNAs. Generally, the presence of dsRNA can trigger an RNAi response. RNAi has been studied in a variety of systems. Fire et al., 1998, Nature, 391, 806, RNAi in C. elegans; Bahramian and Zarbl, 1999, Molecular and Cellular Biology, 19, 274-283 and Wianny and Goetz, 1999, Nature Cell Biol., 2, 70, RNAi mediated by dsRNA in mammalian systems; Hammond et al., 2000, Nature, 404, 293, RNAi in Drosophila cells; Elbashir et al., 2001, Nature, 411, 494, RNAi induced by introduction of duplexes of synthetic 21-nucleotide RNAs in cultured mammalian cells. Such work, along with others, has provided guidance as to the length, structure, chemical composition, and sequence that are helpful in the construction of RNAi molecules in order to mediate RNAi activity. Various publications provide examples of RNAi molecules that can be used as gene expression modulators. Such publications include, U.S. Pat. Nos. 8,993,530, 8,877,917, 8,293,719, 7,947,659, 7,919,473, 7,790,878, 7,737,265, 7,592,322; and U.S. Publication Nos. 20150197746, 20140350071, 20140315835, 20130156845 and 20100267805, the teaching related to the types of RNAi molecules as well as their production are incorporated herein by reference.

Aptamers can bind various protein targets and disrupt the interactions of those proteins with other proteins. Accordingly, the gene expression modulator may be an aptamer, and the gene expression modulating transgene can encode such an aptamer. Aptamers may be selected for their ability to prevent transcription of a gene by specifically binding the DNA-binding sites of regulatory proteins. PCT Publication Nos. WO 98/29430 and WO 00/20040 provides examples of aptamers that were used to modulate gene expression; and U.S. Publication No. 20060128649 also provide examples of such aptamers, the aptamers of each of which are incorporated herein by reference.

As a further example, the gene expression modulatory may be a triplex oligomer. Such a molecule can stall transcription. Generally, this is known as the triplex strategy as the oligomer winds around double-helical DNA, forming a three-strand helix. Such molecules can be designed to recognize a unique site on a chosen gene (Maher, et al., Antisense Res. and Dev., 1(3):227, 1991; Helene, C., Anticancer Drug Design, 6(6):569, 1991).

The viral transfer vectors provided herein may also be used for exon skipping. In such embodiments, the transgene of the viral transfer vector is an exon skipping transgene. Such a transgene encodes an antisense oligonucleotide or other agent that can generate exon skipping. Antisense oligonucleotides may interfere with splice sites or regulatory elements within an exon to lead to truncated, partially functional, protein despite the presence of a genetic mutation. Additionally, antisense oligonucleotides may be mutation-specific and bind to a mutation site in the pre-messenger RNA to induce exon skipping. Antisense oligonucleotides for exon skipping are known in the art and are generally referred to as AONs. Such AONs include snRNA. Examples of antisense oligonucleotides, methods to design them and related production methods can be found, for example, in U.S. Publication Nos. 20150225718, 20150152415, 20150140639, 20150057330, 20150045415, 20140350076, 20140350067, and 20140329762, the AONs of which as well as the described related methods, such as methods of designing and producing the AONs, are incorporated herein by reference in their entirety.

Any one of the methods provided herein may be used to result in exon skipping in cells of a subject in need thereof. The subject may have any disease or disorder in which exon skipping would provide a benefit, and an antisense oligonucleotide can be designed based on an appropriate protein (where exon skipping during its expression would be a benefit) related to such a disease or disorder. Examples of disease and disorders and related proteins are provided herein. In some embodiments of any one of the methods or compositions provided herein, the subject has any one of the dystrophies described herein, such as muscular dystrophy (e.g., Duchenne's muscular dystrophy). Accordingly, in some embodiments of any one of the methods or compositions provided herein the exon skipping transgene encodes an antisense oligonucleotide or other agent that can result in exon skipping in any one of the proteins provided herein that are associated with any one of the dystrophies also provided herein. In some embodiments of any one of the methods or compositions provided herein, the antisense oligonucleotide or other agent can result in exon skipping in dystrophin.

The sequence of a transgene may also include an expression control sequence. Expression control DNA sequences include promoters, enhancers, and operators, and are generally selected based on the expression systems in which the expression construct is to be utilized. In some embodiments, promoter and enhancer sequences are selected for the ability to increase gene expression, while operator sequences may be selected for the ability to regulate gene expression. The transgene may also include sequences that facilitate, and preferably promote, homologous recombination in a host cell. The transgene may also include sequences that are necessary for replication in a host cell.

Exemplary expression control sequences include promoter sequences, e.g., cytomegalovirus promoter; Rous sarcoma virus promoter; and simian virus 40 promoter; as well as any other types of promoters that are disclosed elsewhere herein or are otherwise known in the art. Generally, promoters are operatively linked upstream (i.e., 5′) of the sequence coding for a desired expression product. The transgene also may include a suitable polyadenylation sequence (e.g., the SV40 or human growth hormone gene polyadenylation sequence) operably linked downstream (i.e., 3′) of the coding sequence.

### Viral Vectors

Viruses have evolved specialized mechanisms to transport their genomes inside the cells that they infect; viral vectors based on such viruses can be tailored to transduce cells to specific applications. Examples of viral vectors that may be used as provided herein are known in the art or described herein. Suitable viral vectors include, for instance, retroviral vectors, lentiviral vectors, herpes simplex virus (HSV)-based vectors, adenovirus-based vectors, adeno-associated virus (AAV)-based vectors, and AAV-adenoviral chimeric vectors.

The viral transfer vectors provided herein may be based on a retrovirus. Retrovirus is a single-stranded positive sense RNA virus capable of infecting a wide variety of host cells. Upon infection, the retroviral genome integrates into the genome of its host cell, using its own reverse transcriptase enzyme to produce DNA from its RNA genome. The viral DNA is then replicated along with host cell DNA, which translates and transcribes the viral and host genes. A retroviral vector can be manipulated to render the virus replication-incompetent. As such, retroviral vectors are thought to be particularly useful for stable gene transfer in vivo. Examples of retroviral vectors can be found, for example, in U.S. Publication Nos. 20120009161, 20090118212, and 20090017543, the viral vectors and methods of their making being incorporated by reference herein in their entirety.

Lentiviral vectors are examples of retroviral vectors that can be used for the production of a viral transfer vector as provided herein. Lentiviruses have the ability to infect non-dividing cells, a property that constitute a more efficient method of a gene delivery vector (see, e.g., Durand et al., Viruses. 2011 February; 3(2): 132-159). Examples of lentiviruses include HIV (humans), simian immunodeficiency virus (SIV), feline immunodeficiency virus (FIV), equine infectious anemia virus (EIAV) and visna virus (ovine lentivirus). Unlike other retroviruses, HIV-based vectors are known to incorporate their passenger genes into non-dividing cells. Examples of lentiviral vectors can be found, for example, in U.S. Publication Nos. 20150224209, 20150203870, 20140335607, 20140248306, 20090148936, and 20080254008, the viral vectors and methods of their making being incorporated by reference herein in their entirety.

Herpes simplex virus (HSV)-based viral vectors are also suitable for use as provided herein. Many replication-deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb. For a description of HSV-based vectors, see, for example, U.S. Pat. Nos. 5,837,532, 5,846,782, 5,849,572, and 5,804,413, and International Patent Applications WO 91/02788, WO 96/04394, WO 98/15637, and WO 99/06583, the description of which viral vectors and methods of their making being incorporated by reference in its entirety.

Adenoviruses (Ads) are nonenveloped viruses that can transfer DNA in vivo to a variety of different target cell types. The virus can be made replication-deficient by deleting select genes required for viral replication. The expendable non-replication-essential E3 region is also frequently deleted to allow additional room for a larger DNA insert. Viral transfer vectors can be based on adenoviruses. Adenoviral transfer vectors can be produced in high titers and can efficiently transfer DNA to replicating and non-replicating cells. Unlike lentivirus, adenoviral DNA does not integrate into the genome and therefore is not replicated during cell division, instead they replicate in the nucleus of the host cell using the host's replication machinery.

The adenovirus on which a viral transfer vector may be based may be from any origin, any subgroup, any subtype, mixture of subtypes, or any serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087. Any adenovirus, even a chimeric adenovirus, can be used as the source of the viral genome for an adenoviral vector. For example, a human adenovirus can be used as the source of the viral genome for a replication-deficient adenoviral vector. Further examples of adenoviral vectors can be found in U.S. Publication Nos. 20150093831, 20140248305, 20120283318, 20100008889, 20090175897 and 20090088398, the description of which viral vectors and methods of their making being incorporated by reference in its entirety.

The viral transfer vectors provided herein can also be based on adeno-associated viruses (AAVs). AAV vectors have been of particular interest for use in therapeutic applications such as those described herein. AAV is a DNA virus, which is not known to cause human disease. Generally, AAV requires co-infection with a helper virus (e.g., an adenovirus or a herpes virus), or expression of helper genes, for efficient replication. AAVs have the ability to stably infect host cell genomes at specific sites, making them more predictable than retroviruses; however, generally, the cloning capacity of the vector is 4.9 kb. AAV vectors that have been used in gene therapy applications generally have had approximately 96% of the parental genome deleted, such that only the terminal repeats (ITRs), which contain recognition signals for DNA replication and packaging, remain. For a description of AAV-based vectors, see, for example, U.S. Pat. Nos. 8,679,837, 8,637,255, 8,409,842, 7,803,622, and 7,790,449, and U.S. Publication Nos. 20150065562, 20140155469, 20140037585, 20130096182, 20120100606, and 20070036757, the viral vectors of which and methods or their making being incorporated herein by reference in their entirety. The AAV vectors may be recombinant AAV vectors. The AAV vectors may also be self-complementary (sc) AAV vectors, which are described, for example, in U.S. Patent Publications 2007/01110724 and 2004/0029106, and U.S. Pat. Nos. 7,465,583 and 7,186,699, the vectors and methods of production of which are herein incorporated by reference.

The adeno-associated virus on which a viral transfer vector may be of any serotype or a mixture of serotypes. AAV serotypes include AAV1, AAV 2, AAV3, AAV4, AAV5, AAV6, AAV7, AAV8, AAV9, AAV10, and AAV11. For example, when the viral transfer vector is based on a mixture of serotypes, the viral transfer vector may contain the capsid signal sequences taken from one AAV serotype (for example selected from any one of AAV serotypes 1, 2, 3, 4, 5, 6, 7, 8, 9, 10, and 11) and packaging sequences from a different serotype (for example selected from any one of AAV serotypes 1, 2, 3, 4, 5, 6, 7, 8, 9, 10, and 11). In some embodiments of any one of the methods or compositions provided herein, therefore, the AAV vector is an AAV 2/8 vector. In other embodiments of any one of the methods or compositions provided herein, the AAV vector is an AAV 2/5 vector.

The viral transfer vectors provided herein may also be based on an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral transfer vectors for transgene delivery. Pseudotyped viruses may be formed by combining alphaviral envelope glycoproteins and retroviral capsids. Examples of alphaviral vectors can be found in U.S. Publication Nos. 20150050243, 20090305344, and 20060177819; the vectors and methods of their making are incorporated herein by reference in their entirety.

### Anti-IgM Agents

Anti-IgM agents are any agent that reduces the production of IgM, e.g., IgM antibodies. IgM antibodies are produced by B cells. While IgG antibodies are primarily produced in response to T cell-dependent activation of B cells, IgM antibodies are primarily produced in response to T cell-independent B cell activation, such as occurs in response to infection with viral vectors.

Anti-IgM agents include, but are not limited to, IgM antagonist antibodies or antigen-binding fragments thereof that specifically bind to CD10, CD19, CD20, CD22, CD27, CD34, CD40, CD79a, CD79b, CD123, CD179b, FLT-3, ROR1, BR3, BAFF, or B7RP-1; IL21 modulating agents, e.g., IL-21 and IL-21 receptor antagonists; tyrosine kinase inhibitors, e.g., Syk inhibitors, BTK inhibitors, SRC protein tyrosine kinase inhibitors; PI3K inhibitors; PKC inhibitors; APRIL antagonists, e.g., TACI-Ig; mizoribine; tofacitinib; and tetracyclines.

### IgM Antagonist Antibodies

In some embodiments, the anti-IgM agent is an IgM antagonist antibody or antigen-binding fragment thereof. In some embodiments, the antibody targets a cell surface molecule on a B cell and binding of the antibody recruits the subject's immune system to attack and kill the B cell. In some embodiments, the antibody or antigen-binding fragment thereof specifically binds to CD10, CD19, CD20, CD22, CD27, CD34, CD40, CD79a, CD79b, CD123, CD179b, FLT-3, ROR1, BR3, BAFF, or B7RP-1.

In some embodiments, the antibody is an anti-CD10 antibody, e.g., an antibody that specifically binds CD10. Exemplary anti-CD10 antibodies include, but are not limited to, J5. In some embodiments, the antibody is an anti-CD27 antibody, e.g., an antibody that specifically binds CD27. CD27 is a member of the TNF receptor superfamily. In some embodiments, the antibody is an anti-CD34 antibody, e.g., an antibody that specifically binds CD34. In some embodiments, the antibody is an anti-CD79a antibody, e.g., an antibody that specifically binds CD79a. In some embodiments, the antibody is an anti-CD79b antibody, e.g., an antibody that specifically binds CD79b. Exemplary anti-CD79b antibodies include, but are not limited to, polatuzumab vedotin. In some embodiments, the antibody is an anti-CD123 antibody, e.g., an antibody that specifically binds CD123. Exemplary anti-CD123 antibodies include, but are not limited to, KHK2823 and CSL362. In some embodiments, the antibody is an anti-CD179b antibody, e.g., an antibody that specifically binds CD179b. In some embodiments, the antibody is an anti-FLT-3 antibody, e.g., an antibody that specifically binds FLT-3. Exemplary anti-FLT-3 antibodies include, but are not limited to, sorafenib and quizartinib. In some embodiments, the antibody is an anti-ROR1 antibody, e.g., an antibody that specifically binds ROR1. Exemplary anti-ROR1 antibodies include, but are not limited to, cirmtuzumab. In some embodiments, the antibody is an anti-BR3 antibody, e.g., an antibody that specifically binds BR3. In some embodiments, the antibody is an anti-B7RP-1 antibody, e.g., an antibody that specifically binds B7RP-1. Exemplary anti-B7RP-1 antibodies include, but are not limited to, prezalumab.

In some embodiments, the antibody is an anti-CD19 antibody, e.g., an antibody that specifically binds CD19. Exemplary anti-CD19 antibodies include, but are not limited to, MOR00208 (MorphoSysAG).

In some embodiments, the antibody is an anti-CD20 antibody, e.g., an antibody that specifically binds CD20. Exemplary anti-CD20 antibodies include, but are not limited to, rituximab, obinutuzumab, ocrelizumab, ofatumumab, iodine 131 tositumomab (Bexxar), ibritumomab, hyaluronidase/rituximab, and ibritumomab.

In some embodiments, the antibody is an anti-CD22 antibody, e.g., an antibody that specifically binds CD22. Exemplary anti-CD22 antibodies include, but are not limited to, epratuzumab and moxetumomab.

In some embodiments, the antibody is an anti-CD40 antibody, e.g., an antibody that specifically binds CD40. Exemplary anti-CD40 antibodies include, but are not limited to, ABBV-927 (Abbvie) and APX005M (Apexigen).

In some embodiments, the antibody is an anti-BAFF antibody or antigen-binding fragment thereof. BAFF, B cell activation factor (B lymphocyte stimulator), is an important cytokine for the generation and maintenance of B cells. BAFF has multiple receptors, which play a role in transmitting signals to different classes of B cells, such as BAFF-R, which is selective and important in early B-cell homeostasis and T-reg function and B-cell maturation antigen (BCMA), which is restricted to antibody-producing cells and is important for plasma cell longevity. Anti-BAFF antibodies, such as Belimumab, can include agents that specifically bind BAFF. Anti-BAFF antibodies may interfere with the interaction between BAFF and its receptors, such as BAFF-R and BCMA (B cell maturation antigen). Anti-BAFF antibodies are commercially available and one skilled in the art would be able to acertain whether a certain agent is an anti-BAFF antibody. Any one of the anti-BAFF antibodies described herein or otherwise known, or antigen-binding fragments thereof, may be used in any one of the methods provided or be comprised in any one of the compositions or kits provided.

In some embodiments, the antibody or antigen-binding fragment thereof as described herein can bind and inhibit the activity of its target at least 50% (e.g., 60%, 70%, 80%, 90%, 95% or greater). The inhibitory activity of any of the antibodies or antigen-binding fragments thereof described herein can be determined by routine methods known in the art, for example, with an ELISA. Furthermore, binding affinity (or binding specificity) can be determined by a variety of methods including equilibrium dialysis, equilibrium binding, gel filtration, ELISA, surface plasmon resonance, or spectroscopy (e.g., using a fluorescence assay).

As used herein, “antibody” refers to a glycoprotein comprising at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “antigen-binding fragment” of an antibody refers to one or more portions of an antibody that retain the ability to bind specifically to an antigen. The antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, V and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference, as well as by other techniques known to those with skill in the art. The fragments can be screened for utility in the same manner as are intact antibodies.

In embodiments of any one of the methods or compositions or kits provided herein, the antibody or antigen-binding fragment thereof may be those produced by engineered sequences based on an antibody or antigen-binding fragment thereof.

Examples of antibodies described herein are commercially available and one skilled in the art would be able to acertain whether a certain agent is a CD10, CD19, CD20, CD22, CD27, CD34, CD40, CD79a, CD79b, CD123, CD179b, FLT-3, ROR1, BR3, BAFF, or B7RP-1 antibody. Any one of the antibodies described herein or otherwise known, or antigen-binding fragments thereof, may be used in any one of the methods provided or be comprised in any one of the compositions or kits provided.

### Tyrosine Kinase Inhibitors

In some embodiments, the anti-IgM agent is a tyrosine kinase inhibitor, e.g., a syk inhibitor, a BTK inhibitor, or a SRC protein tyrosine kinase inhibitor.

In some embodiments, the anti-IgM agent is a syk inhibitor. Exemplary syk inhibitors include, but are not limited to, fostamatinib (R788), entospletinib (GS-9973), cerdulatinib (PRT062070), TAK-659, entospletinib, and nilvadipine.

In some embodiments, the anti-IgM agent is a BTK inhibitor. BTK inhibitors include small molecule inhibitors of BTK, antibodies to BTK, and antisense oligomers and RNAi inhibitors that reduce the expression of BTK. Exemplary BTK inhibitors include, but are not limited to, ibrutinib, AVL-292, CC-292, ONO-4059, ACP-196, PCI-32765, Acalabrutinib, GS-4059, spebrutinib, BGB-3111, and HM71224.

In some embodiments, the anti-IgM agent is a SRC protein tyrosine kinase inhibitor. SRC inhibitors include small molecule inhibitors of SRC, antibodies to SRC, and antisense oligomers and RNAi inhibitors that reduce the expression of SRC. Exemplary SRC protein tyrosine kinase inhibitors include, but are not limited to, dasatinib.

In some embodiments, the anti-IgM agent is an anti-BAFF agent. An anti-BAFF agent refers to any agent, small molecules, antibodies, peptides, or nucleic acids, that is known to reduce the production, or levels of, or activity of BAFF. In some embodiments, an anti-BAFF agent is an anti-BAFF antibody described herein. Exemplary anti-BAFF agents include, but are not limited to, TACI-Ig and soluble BAFF receptor.

In some embodiments, the anti-IgM agent is a PI3K inhibitor. PI3 kinases include, but are not limited to, PIK3CA, PIK3CB, PIK3CG, PIK3CD, PIK3R1, PIK3R2, PIK3R3, PIK3R4, PIK3R5, PIK3R6, PIK3C2A, PIK3C2B, PIK3C2G, and PIK3C3. PI3K inhibitors include small molecule inhibitors of PI3K, antibodies to PI3K, and antisense oligomers and RNAi inhibitors that reduce the expression of PI3K. Exemplary PI3K inhibitors include, but are not limited to, GS-1101, idelalisib, duvelisib, TGR-1202, AMG-319, copanlisib, wortmannin, LY294002, IC486068 and IC87114 (ICOS Corporation), and GDC-0941.

In some embodiments, the anti-IgM agent is a PKC inhibitor. PKC inhibitors include small molecule inhibitors of PKC, antibodies to PKC, and antisense oligomers and RNAi inhibitors that reduce the expression of PKC. Exemplary PKC inhibitors include, but are not limited to, enzastaurin.

In some embodiments, the anti-IgM agent is an APRIL antagonist. APRIL antagonists include small molecule inhibitors of APRIL, antibodies to APRIL, and antisense oligomers and RNAi inhibitors that reduce the expression of APRIL. In some embodiments, the APRIL antagonist is an antibody. Exemplary anti-APRIL antibodies include, but are not limited to, BION-1301 (Aduro Biotech, Inc.) In some embodiments, the anti-IgM agent is TACI-Ig, Atacicept.

In some embodiments, the anti-IgM agent is an IL-21 modulating agent. Exemplary IL-21 inhibitors include, but are not limited to, NNC0114 (NovoNordisk). In some embodiments, an IL-21 modulating agent is an IL-21 receptor antagonist. IL-21 receptor antagonists include small molecule inhibitors of the IL-21 receptor, antibodies to the IL-21 receptor, and antisense oligomers and RNAi inhibitors that reduce the expression of the IL-21 receptor. Exemplary IL-21 receptor inhibitors include, but are not limited to, ATR-107(Pfizer). Exemplary IL-21 antagonists include, but are not limited to, NNC0114 (NovoNordisk). In some embodiments, the anti-IgM agent is an IL-21 receptor antagonist. Exemplary IL-21 receptor antagonists include, but are not limited to ATR-107(Pfizer).

In some embodiments, the anti-IgM agent is mizoribine.

In some embodiments, the anti-IgM agent is tofacitinib.

In some embodiments, the anti-IgM agent is a tetracycline. Exemplary tetracyclines include, but are not limited to, chlortetracycline, oxytetracycline, demethylchlortetracycline, rolitetracycline, limecycline, clomocycline, methacycline, doxycycline, minocycline, and tertiary-butylglycylamidominocycline.

### Synthetic Nanocarriers Comprising an Immunosuppressant

A wide variety of other synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size or shape so that each synthetic nanocarrier has similar properties. For example, at least 80%, at least 90%, or at least 95% of the synthetic nanocarriers of any one of the compositions or methods provided, based on the total number of synthetic nanocarriers, may have a minimum dimension or maximum dimension that falls within 5%, 10%, or 20% of the average diameter or average dimension of the synthetic nanocarriers.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, synthetic nanocarriers can comprise one or more polymers. In some embodiments, the synthetic nanocarriers comprise one or more polymers that is a non-methoxy-terminated, pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated, pluronic polymers. In some embodiments, all of the polymers that make up the synthetic nanocarriers are non-methoxy-terminated, pluronic polymers. In some embodiments, the synthetic nanocarriers comprise one or more polymers that is a non-methoxy-terminated polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated polymers. In some embodiments, all of the polymers that make up the synthetic nanocarriers are non-methoxy-terminated polymers. In some embodiments, the synthetic nanocarriers comprise one or more polymers that do not comprise pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers do not comprise pluronic polymer. In some embodiments, all of the polymers that make up the synthetic nanocarriers do not comprise pluronic polymer. In some embodiments, such a polymer can be surrounded by a coating layer (e.g., liposome, lipid monolayer, micelle, etc.). In some embodiments, elements of the synthetic nanocarriers can be attached to the polymer.

Immunosuppressants can be coupled to the synthetic nanocarriers by any of a number of methods. Generally, the attaching can be a result of bonding between the immunosuppressants and the synthetic nanocarriers. This bonding can result in the immunosuppressants being attached to the surface of the synthetic nanocarriers and/or contained (encapsulated) within the synthetic nanocarriers. In some embodiments, however, the immunosuppressants are encapsulated by the synthetic nanocarriers as a result of the structure of the synthetic nanocarriers rather than bonding to the synthetic nanocarriers. In preferable embodiments, the synthetic nanocarrier comprises a polymer as provided herein, and the immunosuppressants are attached to the polymer.

When attaching occurs as a result of bonding between the immunosuppressants and synthetic nanocarriers, the attaching may occur via a coupling moiety. A coupling moiety can be any moiety through which an immunosuppressant is bonded to a synthetic nanocarrier. Such moieties include covalent bonds, such as an amide bond or ester bond, as well as separate molecules that bond (covalently or non-covalently) the immunosuppressant to the synthetic nanocarrier. Such molecules include linkers or polymers or a unit thereof. For example, the coupling moiety can comprise a charged polymer to which an immunosuppressant electrostatically binds. As another example, the coupling moiety can comprise a polymer or unit thereof to which it is covalently bonded.

In preferred embodiments, the synthetic nanocarriers comprise a polymer as provided herein. These synthetic nanocarriers can be completely polymeric or they can be a mix of polymers and other materials.

In some embodiments, the polymers of a synthetic nanocarrier associate to form a polymeric matrix. In some of these embodiments, a component, such as an immunosuppressant, can be covalently associated with one or more polymers of the polymeric matrix. In some embodiments, covalent association is mediated by a linker. In some embodiments, a component can be noncovalently associated with one or more polymers of the polymeric matrix. For example, in some embodiments, a component can be encapsulated within, surrounded by, and/or dispersed throughout a polymeric matrix. Alternatively or additionally, a component can be associated with one or more polymers of a polymeric matrix by hydrophobic interactions, charge interactions, van der Waals forces, etc. A wide variety of polymers and methods for forming polymeric matrices therefrom are known conventionally.

Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments, the polymer comprises a polyester, polycarbonate, polyamide, or polyether, or unit thereof. In other embodiments, the polymer comprises poly(ethylene glycol) (PEG), polypropylene glycol, poly(lactic acid), poly(glycolic acid), poly(lactic-co-glycolic acid), or a polycaprolactone, or unit thereof. In some embodiments, it is preferred that the polymer is biodegradable. Therefore, in these embodiments, it is preferred that if the polymer comprises a polyether, such as poly(ethylene glycol) or polypropylene glycol or unit thereof, the polymer comprises a block-co-polymer of a polyether and a biodegradable polymer such that the polymer is biodegradable. In other embodiments, the polymer does not solely comprise a polyether or unit thereof, such as poly(ethylene glycol) or polypropylene glycol or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids. Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids. In embodiments, the synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

Any immunosuppressant as provided herein can be, in some embodiments, coupled to synthetic nanocarriers. Immunosuppressants include, but are not limited to, statins; mTOR inhibitors, such as rapamycin or a rapamycin analog (“rapalog”); TGF-β signaling agents; TGF-β receptor agonists; histone deacetylase (HDAC) inhibitors; corticosteroids; inhibitors of mitochondrial function, such as rotenone; P38 inhibitors; NF-κβ inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cyclosporine A, siRNAs targeting cytokines or cytokine receptors and the like.

Examples of mTOR inhibitors include rapamycin and analogs thereof (e.g., CCL-779, RAD001, AP23573, C20-methallylrapamycin (C20-Marap), C16-(S)-butylsulfonamidorapamycin (C16-BSrap), C16-(S)-3-methylindolerapamycin (C16-iRap) (Bayle et al. Chemistry & Biology 2006, 13:99-107)), AZD8055, BEZ235 (NVP-BEZ235), chrysophanic acid (chrysophanol), deforolimus (MK-8669), everolimus (RAD0001), KU-0063794, PI-103, PP242, temsirolimus, and WYE-354 (available from Selleck, Houston, Tex., USA).

Examples of NF (e.g., NK-κβ) inhibitors include IFRD1, 2-(1,8-naphthyridin-2-yl)-Phenol, 5-aminosalicylic acid, BAY 11-7082, BAY 11-7085, CAPE (Caffeic Acid Phenethylester), diethylmaleate, IKK-2 Inhibitor IV, IMD 0354, lactacystin, MG-132 [Z-Leu-Leu-Leu-CHO], NFκB Activation Inhibitor III, NF-κB Activation Inhibitor II, JSH-23, parthenolide, Phenylarsine Oxide (PAO), PPM-18, pyrrolidinedithiocarbamic acid ammonium salt, QNZ, RO 106-9920, rocaglamide, rocaglamide AL, rocaglamide C, rocaglamide I, rocaglamide J, rocaglaol, (R)-MG-132, sodium salicylate, triptolide (PG490), and wedelolactone.

“Rapalog”, as used herein, refers to a molecule that is structurally related to (an analog) of rapamycin (sirolimus). Examples of rapalogs include, without limitation, temsirolimus (CCI-779), everolimus (RAD001), ridaforolimus (AP-23573), and zotarolimus (ABT-578). Additional examples of rapalogs may be found, for example, in WO Publication WO 1998/002441 and U.S. Pat. No. 8,455,510, the rapalogs of which are incorporated herein by reference in their entirety.

Further immunosuppressants are known to those of skill in the art, and the invention is not limited in this respect. In embodiments of any one of the methods, compositions or kits provided, the immunosuppressant may comprise any one of the agents as provided herein.

Compositions according to the invention can comprise pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, compositions are suspended in sterile saline solution for injection together with a preservative.

### D. Methods of Using and Making the Compositions

Viral transfer vectors can be made with methods known to those of ordinary skill in the art or as otherwise described herein. For example, viral transfer vectors can be constructed and/or purified using the methods set forth, for example, in U.S. Pat. No. 4,797,368 and Laughlin et al., Gene, 23, 65-73 (1983).

As an example, replication-deficient adenoviral vectors can be produced in complementing cell lines that provide gene functions not present in the replication-deficient adenoviral vectors, but required for viral propagation, at appropriate levels in order to generate high titers of viral transfer vector stock. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons). Construction of complementing cell lines involve standard molecular biology and cell culture techniques, such as those described by Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Complementing cell lines for producing adenoviral vectors include, but are not limited to, 293 cells (described in, e.g., Graham et al., J. Gen. Virol., 36, 59-72 (1977)), PER.C6 cells (described in, e.g., International Patent Application WO 97/00326, and U.S. Pat. Nos. 5,994,128 and 6,033,908), and 293-ORF6 cells (described in, e.g., International Patent Application WO 95/34671 and Brough et al., J. Virol., 71, 9206-9213 (1997)). In some instances, the complementing cell will not complement for all required adenoviral gene functions. Helper viruses can be employed to provide the gene functions in trans that are not encoded by the cellular or adenoviral genomes to enable replication of the adenoviral vector. Adenoviral vectors can be constructed, propagated, and/or purified using the materials and methods set forth, for example, in U.S. Pat. Nos. 5,965,358, 5,994,128, 6,033,908, 6,168,941, 6,329,200, 6,383,795, 6,440,728, 6,447,995, and 6,475,757, U.S. Patent Application Publication No. 2002/0034735 A1, and International Patent Applications WO 98/53087, WO 98/56937, WO 99/15686, WO 99/54441, WO 00/12765, WO 01/77304, and WO 02/29388, as well as the other references identified herein. Non-group C adenoviral vectors, including adenoviral serotype 35 vectors, can be produced using the methods set forth in, for example, U.S. Pat. Nos. 5,837,511 and 5,849,561, and International Patent Applications WO 97/12986 and WO 98/53087.

AAV vectors may be produced using recombinant methods. Typically, the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, the viral transfer vector may comprise inverted terminal repeats (ITR) of AAV serotypes selected from the group consisting of: AAV1, AAV2, AAV5, AAV6, AAV6.2, AAV7, AAV8, AAV9, AAV10, AAV11 and variants thereof.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell can contain the required component(s) under the control of an inducible promoter. However, the required component(s) may be under the control of a constitutive promoter. The recombinant AAV vector, rep sequences, cap sequences, and helper functions required for producing the rAAV of the invention may be delivered to the packaging host cell using any appropriate genetic element. The selected genetic element may be delivered by any suitable method, including those described herein. Methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

In some embodiments, recombinant AAV vectors may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (comprising a transgene) to be packaged into AAV particles, an AAV helper function vector, and an accessory function vector. Generally, an AAV helper function vector encodes AAV helper function sequences (rep and cap), which function in trans for productive AAV replication and encapsidation. Preferably, the AAV helper function vector supports efficient AAV vector production without generating any detectable wild-type AAV virions (i.e., AAV virions containing functional rep and cap genes). The accessory function vector can encode nucleotide sequences for non-AAV derived viral and/or cellular functions upon which AAV is dependent for replication. The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Lentiviral vectors may be produced using any of a number of methods known in the art. Examples of lentiviral vectors and/or methods of their production can be found, for example, in U.S. Publication Nos. 20150224209, 20150203870, 20140335607, 20140248306, 20090148936, and 20080254008, such lentiviral vectors and methods of production are incorporated herein by reference. As an example, when the lentiviral vector is integration-incompetent, the lentiviral genome further comprises an origin of replication (ori), whose sequence is dependent on the nature of cells where the lentiviral genome has to be expressed. Said origin of replication may be from eukaryotic origin, preferably of mammalian origin, most preferably of human origin. Since the lentiviral genome does not integrate into the cell host genome (because of the defective integrase), the lentiviral genome can be lost in cells undergoing frequent cell divisions; this is particularly the case in immune cells, such as B or T cells. The presence of an origin of replication can be beneficial in some instances. Vector particles may be produced after transfection of appropriate cells, such as 293 T cells, by said plasmids, or by other processes. In the cells used for the expression of the lentiviral particles, all or some of the plasmids may be used to stably express their coding polynucleotides, or to transiently or semi-stably express their coding polynucleotides.

Methods for producing other viral vectors as provided herein are known in the art and may be similar to the exemplified methods above. Moreover, viral vectors are available commercially.

In embodiments, when preparing certain synthetic nanocarriers comprising an immunosuppressant, methods for attaching an immunosuppressant to synthetic nanocarriers may be useful.

In certain embodiments, the attaching can be a covalent linker. In embodiments, immunosuppressants according to the invention can be covalently attached to the external surface via a 1,2,3-triazole linker formed by the 1,3-dipolar cycloaddition reaction of azido groups with immunosuppressant containing an alkyne group or by the 1,3-dipolar cycloaddition reaction of alkynes with immunosuppressants containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component such as an immunosuppressant with the carboxylic acid group of a second component such as the nanocarrier. The amide bond in the linker can be made using any of the conventional amide bond forming reactions with suitably protected amino acids and activated carboxylic acid such N-hydroxysuccinimide-activated ester.

A disulfide linker is made via the formation of a disulfide (S—S) bond between two sulfur atoms of the form, for instance, of R1-S—S—R2. A disulfide bond can be formed by thiol exchange of a component containing thiol/mercaptan group (—SH) with another activated thiol group or a component containing thiol/mercaptan groups with a component containing activated thiol group.

A triazole linker, specifically a 1,2,3-triazole of the form

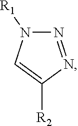

wherein R1 and R2 may be any chemical entities, is made by the 1,3-dipolar cycloaddition reaction of an azide attached to a first component with a terminal alkyne attached to a second component such as the immunosuppressant. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

A thioether linker is made by the formation of a sulfur-carbon (thioether) bond in the form, for instance, of R1-S-R2. Thioether can be made by either alkylation of a thiol/mercaptan (—SH) group on one component with an alkylating group such as halide or epoxide on a second component. Thioether linkers can also be formed by Michael addition of a thiol/mercaptan group on one component to an electron-deficient alkene group on a second component containing a maleimide group or vinyl sulfone group as the Michael acceptor. In another way, thioether linkers can be prepared by the radical thiol-ene reaction of a thiol/mercaptan group on one component with an alkene group on a second component.

A hydrazone linker is made by the reaction of a hydrazide group on one component with an aldehyde/ketone group on the second component.

A hydrazide linker is formed by the reaction of a hydrazine group on one component with a carboxylic acid group on the second component. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component with an aldehyde or ketone group on the second component.

An urea or thiourea linker is prepared by the reaction of an amine group on one component with an isocyanate or thioisocyanate group on the second component.

An amidine linker is prepared by the reaction of an amine group on one component with an imidoester group on the second component.

An amine linker is made by the alkylation reaction of an amine group on one component with an alkylating group such as halide, epoxide, or sulfonate ester group on the second component. Alternatively, an amine linker can also be made by reductive amination of an amine group on one component with an aldehyde or ketone group on the second component with a suitable reducing reagent such as sodium cyanoborohydride or sodium triacetoxyborohydride.

A sulfonamide linker is made by the reaction of an amine group on one component with a sulfonyl halide (such as sulfonyl chloride) group on the second component.

A sulfone linker is made by Michael addition of a nucleophile to a vinyl sulfone. Either the vinyl sulfone or the nucleophile may be on the surface of the nanocarrier or attached to a component.

The component can also be conjugated via non-covalent conjugation methods. For example, a negative charged immunosuppressant can be conjugated to a positive charged component through electrostatic adsorption. A component containing a metal ligand can also be conjugated to a metal complex via a metal-ligand complex.

In embodiments, the component can be attached to a polymer, for example polylactic acid-block-polyethylene glycol, prior to the assembly of a synthetic nanocarrier or the synthetic nanocarrier can be formed with reactive or activatable groups on its surface. In the latter case, the component may be prepared with a group which is compatible with the attachment chemistry that is presented by the synthetic nanocarriers' surface. In other embodiments, a peptide component can be attached to VLPs or liposomes using a suitable linker. A linker is a compound or reagent that capable of coupling two molecules together. In an embodiment, the linker can be a homobifuntional or heterobifunctional reagent as described in Hermanson 2008. For example, an VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a peptide component containing an acid group via the other end of the ADH linker on nanocarrier to produce the corresponding VLP or liposome peptide conjugate.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The component is prepared with the presence of either an alkyne (if the polymer contains an azide) or an azide (if the polymer contains an alkyne) group. The component is then allowed to react with the nanocarrier via the 1,3-dipolar cycloaddition reaction with or without a catalyst which covalently attaches the component to the particle through the 1,4-disubstituted 1,2,3-triazole linker.

If the component is a small molecule it may be of advantage to attach the component to a polymer prior to the assembly of synthetic nanocarriers. In embodiments, it may also be an advantage to prepare the synthetic nanocarriers with surface groups that are used to attach the component to the synthetic nanocarrier through the use of these surface groups rather than attaching the component to a polymer and then using this polymer conjugate in the construction of synthetic nanocarriers.

For detailed descriptions of available conjugation methods, see Hermanson G T “Bioconjugate Techniques”, 2nd Edition Published by Academic Press, Inc., 2008. In addition to covalent attachment the component can be attached by adsorption to a pre-formed synthetic nanocarrier or it can be attached by encapsulation during the formation of the synthetic nanocarrier.

Synthetic nanocarriers may be prepared using a wide variety of methods known in the art. For example, synthetic nanocarriers can be formed by methods such as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

Materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. No. 6,632,671 to Unger issued Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be attached to the synthetic nanocarriers and/or the composition of the polymer matrix.

If synthetic nanocarriers prepared by any of the above methods have a size range outside of the desired range, synthetic nanocarriers can be sized, for example, using a sieve.

Elements of the synthetic nanocarriers may be attached to the overall synthetic nanocarrier, e.g., by one or more covalent bonds, or may be attached by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be attached to components directly or indirectly via non-covalent interactions. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such attachments may be arranged to be on an external surface or an internal surface of a synthetic nanocarrier. In embodiments, encapsulation and/or absorption is a form of attaching.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention may comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, compositions are suspended in sterile saline solution for injection with a preservative.

It is to be understood that the compositions of the invention can be made in any suitable manner, and the invention is in no way limited to compositions that can be produced using the methods described herein. Selection of an appropriate method of manufacture may require attention to the properties of the particular moieties being associated.

In some embodiments, compositions are manufactured under sterile conditions or are terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection.

Administration according to the present invention may be by a variety of routes, including but not limited to intravenous and intraperitoneal routes. The compositions referred to herein may be manufactured and prepared for administration, in some embodiments concomitant administration, using conventional methods.

The compositions of the invention can be administered in effective amounts, such as the effective amounts described elsewhere herein. In some embodiments, the viral transfer vectors and/or synthetic nanocarriers comprising an immunosuppressant and/or anti-IgM agent are present in dosage forms in an amount effective to attenuate an anti-viral transfer vector immune response, such as an IgM response, and/or allow for readministration of a viral transfer vector to a subject and/or increase transgene expression of the viral transfer vector. Dosage forms may be administered at a variety of frequencies. In some embodiments, repeated administration of a viral transfer vector with synthetic nanocarriers comprising an immunosuppressant and an anti-IgM agentis undertaken.

Aspects of the invention relate to determining a protocol for the methods of administration as provided herein. A protocol can be determined by varying at least the frequency, dosage amount of the viral transfer vector, of the synthetic nanocarriers comprising an immunosuppressant and/or of the anti-IgM agent and subsequently assessing a desired or undesired immune response. A preferred protocol for practice of the invention attenuates an immune response against the viral transfer vector, such as an IgM response and/or attenuates another undesired immune response against the viral transfer vector and/or escalates transgene expression. The protocol can comprise at least the frequency of the administration and doses of the viral transfer vector, synthetic nanocarriers comprising an immunosuppressant and anti-IgM agent in some embodiments.

Another aspect of the disclosure relates to kits. In some embodiments, the kit comprises any one or more of the compositions provided herein or any one of the combinations of the compositions provided herein. In some embodiments, the kit comprises one or more compositions comprising a viral transfer vector and/or one or more compositions comprising synthetic nanocarriers comprising an immunosuppressant and/or one or more compositions comprising an anti-IgM agent. Preferably, the composition(s) is/are in an amount to provide any one or more doses as provided herein. The composition(s) can be in one container or in more than one container in the kit. In some embodiments of any one of the kits provided, the container is a vial or an ampoule. In some embodiments of any one of the kits provided, the composition(s) are in lyophilized form each in a separate container or in the same container, such that they may be reconstituted at a subsequent time. In some embodiments of any one of the kits provided, the kit further comprises instructions for reconstitution, mixing, administration, etc. In some embodiments of any one of the kits provided, the instructions include a description of any one of the methods described herein. Instructions can be in any suitable form, e.g., as a printed insert or a label. In some embodiments of any one of the kits provided herein, the kit further comprises one or more syringes or other device(s) that can deliver the composition(s) in vivo to a subject.

### EXAMPLES

### Example 1: Synthetic Nanocarriers Comprising an Immunosuppressant

Synthetic nanocarriers comprising an immunosuppressant, such as rapamycin, can be produced using any method known to those of ordinary skill in the art. Preferably, in some embodiments of any one of the methods, compositions or kits provided herein the synthetic nanocarriers comprising an immunosuppressant are produced by any one of the methods of US Publication No. US 2016/0128986 A1 and US Publication No. US 2016/0128987 A1, the described methods of such production and the resulting synthetic nanocarriers being incorporated herein by reference in their entirety. In any one of the methods, compositions or kits provided herein, the synthetic nanocarriers comprising an immunosuppressant are such incorporated synthetic nanocarriers. Synthetic nanocarriers comprising rapamycin were produced with methods at least similar to these incorporated methods and used in the following Example.

### Example 2: Combination Delivery of Adeno-Associated Virus (AAV) with Synthetic Nanocarriers Comprising an Immunosuppressant and Anti-BAFF Antibody

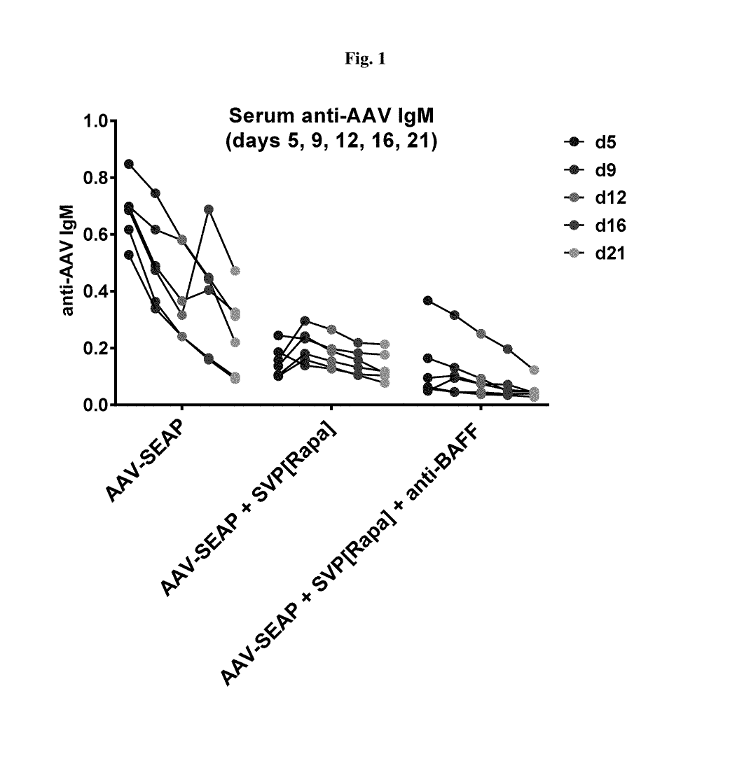

The effect of administering an adeno-associated virus vector with a synthetic nanocarrier comprising an immunosuppressant (rapamycin) and an anti-BAFF antibody was examined. Three treatments were tested: adeno-associated viral vector encoding for secreted alkaline phosphatase (AAV-SEAP) alone, in combination with synthetic nanocarriers comprising rapamycin (AAV-SEAP+SVP[RAPA]), and in combination with an anti-BAFF antibody (AAV-SEAP+SVP[RAPA]+anti-BAFF]). Three groups of six mice were injected one time with identical amounts of one of the three treatments described above. Injections were administered intravenously (i.v.) for AAV-SEAP and SVP[RAPA] and intraperitoneally (i.p.) for anti-BAFF. Whole blood was collected and processed to isolate serum from each subject on days 5, 9, 12, 16, and 21 post-injection. Serum IgM directed toward plate-bound AAV was determined using an ELISA. Naïve serum was used as the negative baseline level. As shown in FIG. 1, the administration of AAV-SEAP in combination with synthetic nanocarriers comprising rapamycin and the anti-BAFF antibody resulted in a reduction of serum anti-AAV IgM levels compared to the other two groups. By days 16 and 21, anti-AAV immunity was nearly abolished in a number of mice receiving the combination of AAV vector and synthetic nanocarriers comprising rapamycin and the anti-BAFF antibody.

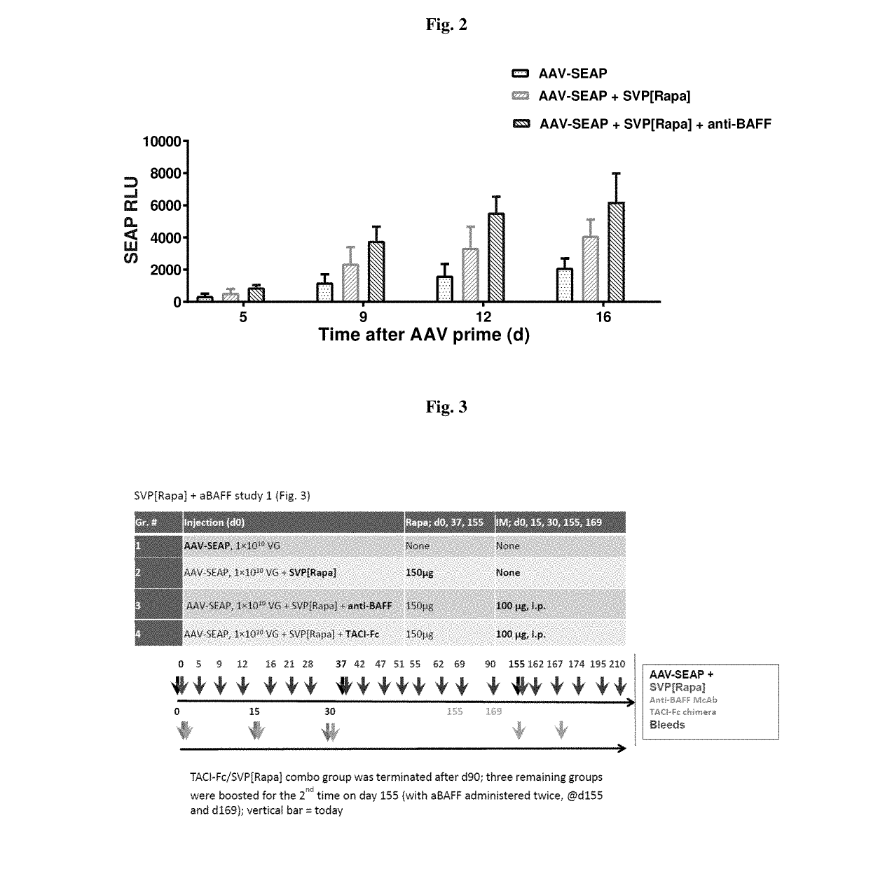

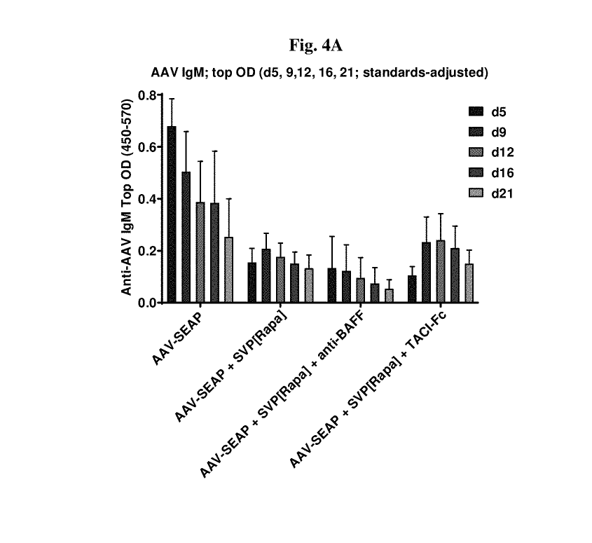

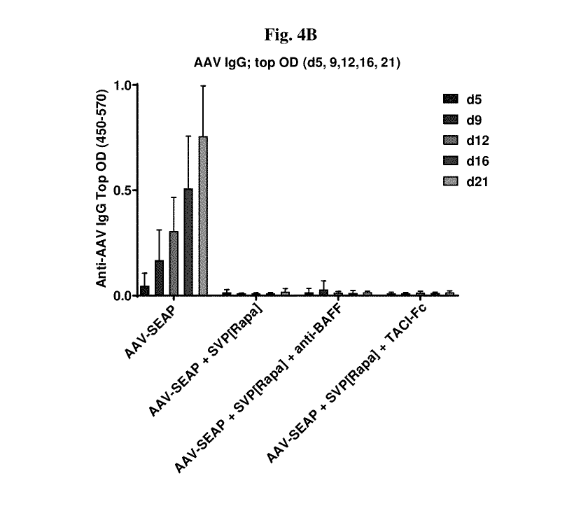

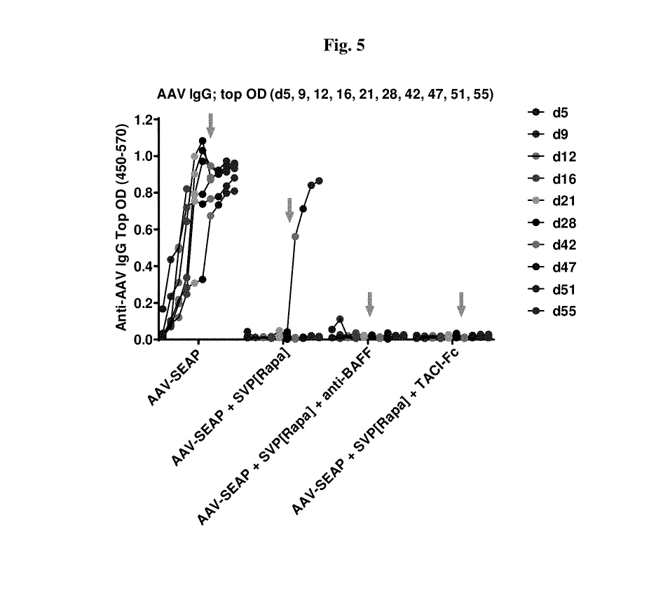

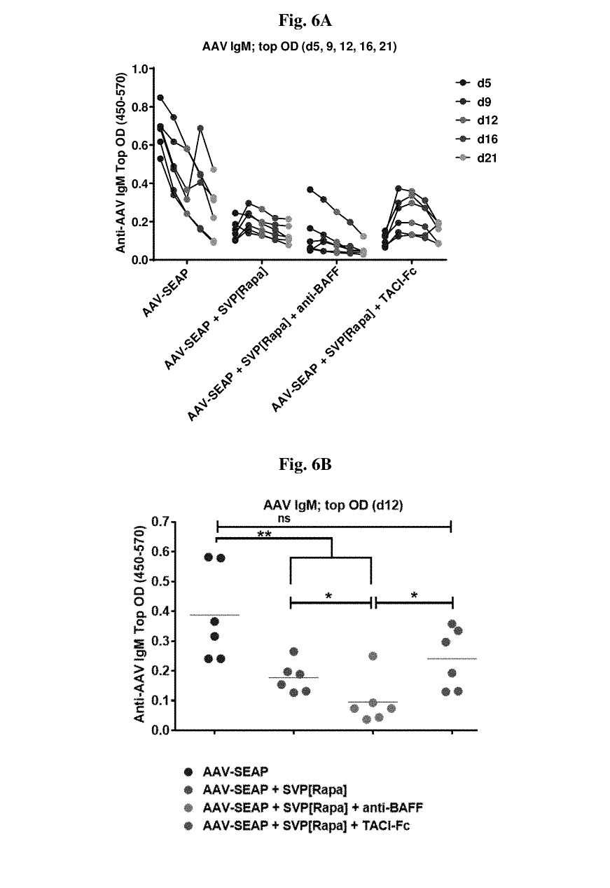

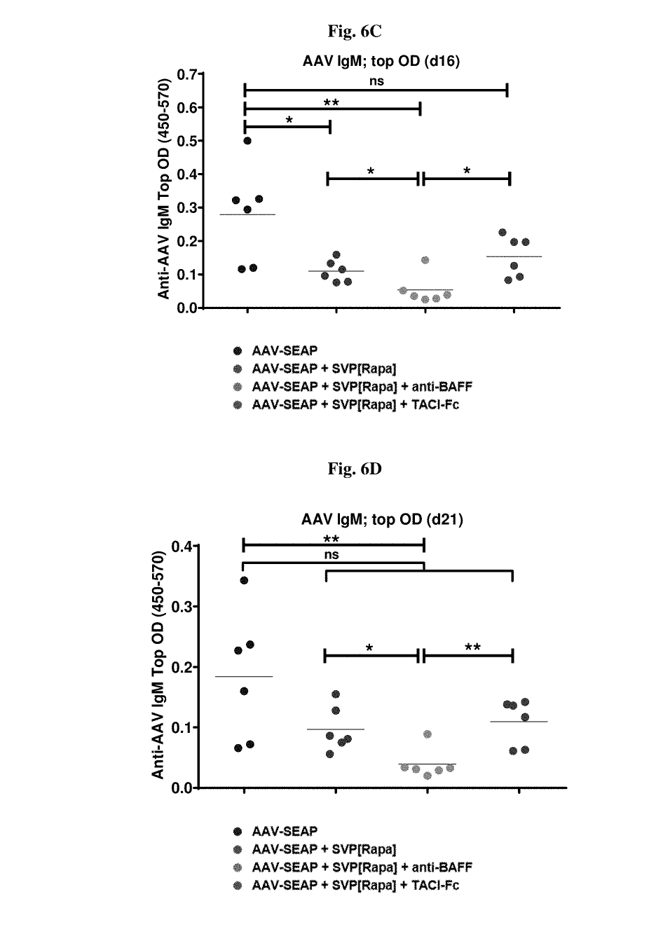

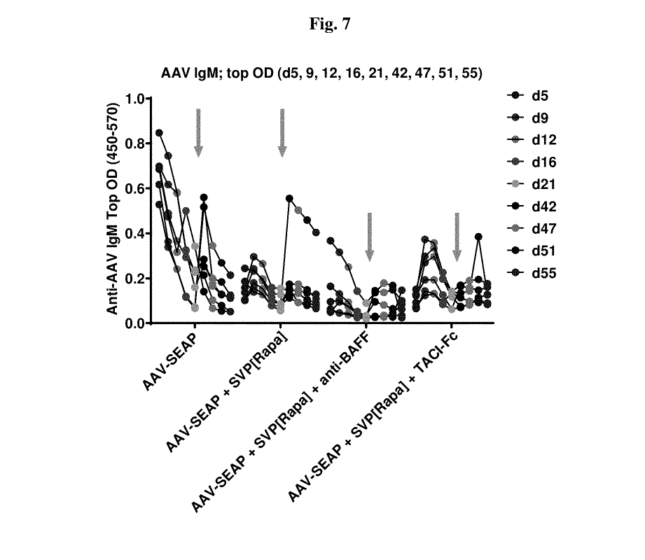

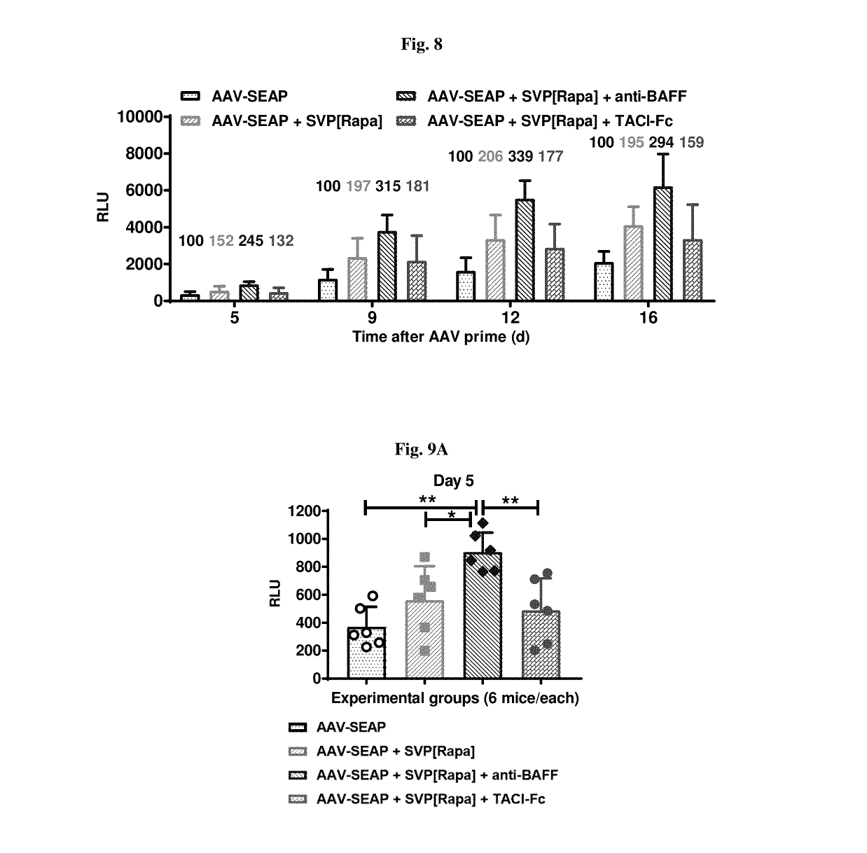

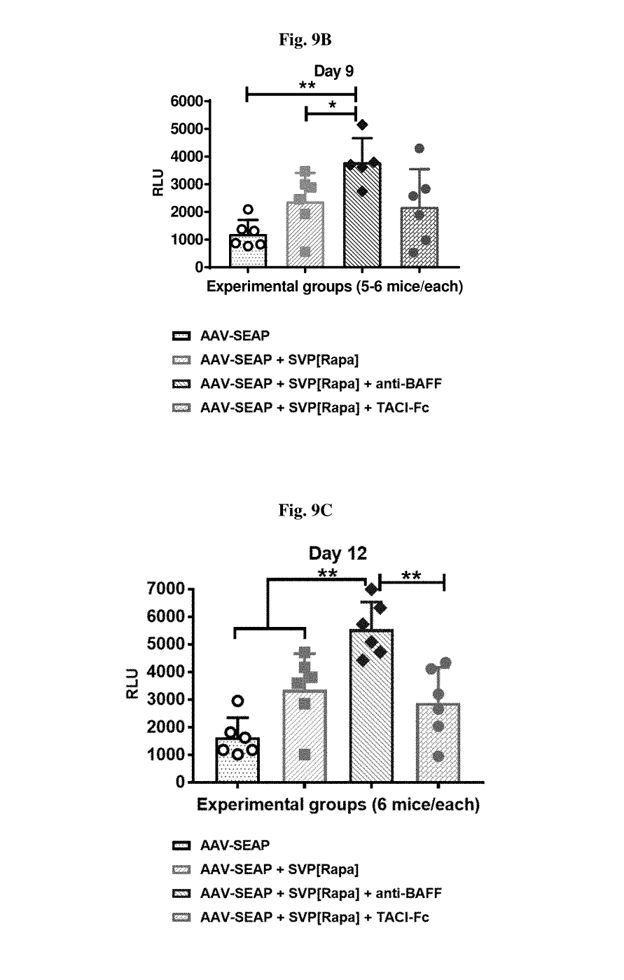

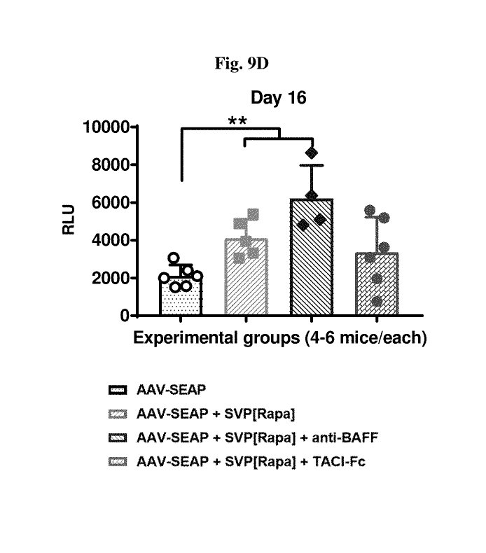

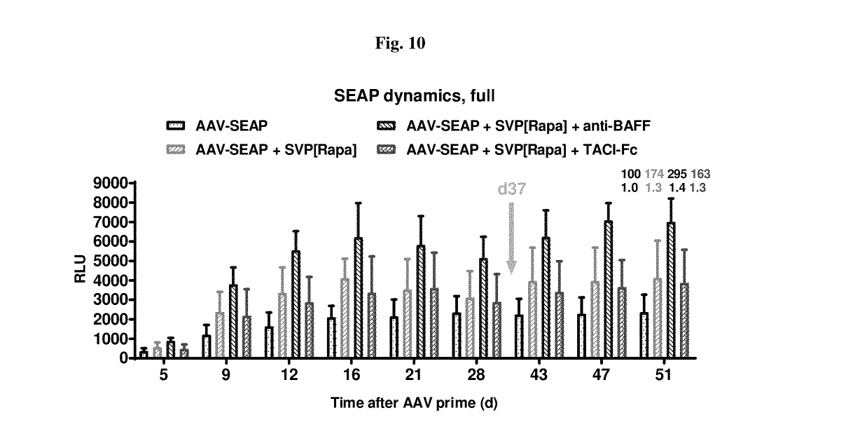

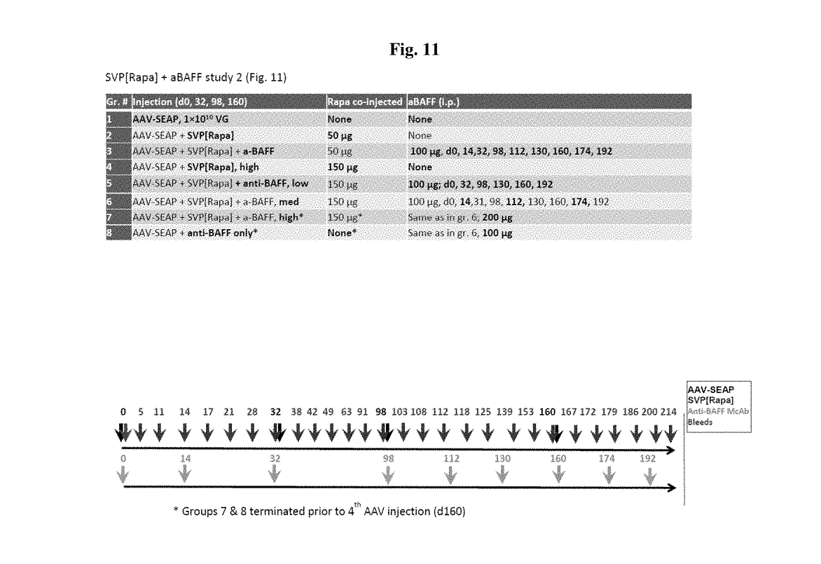

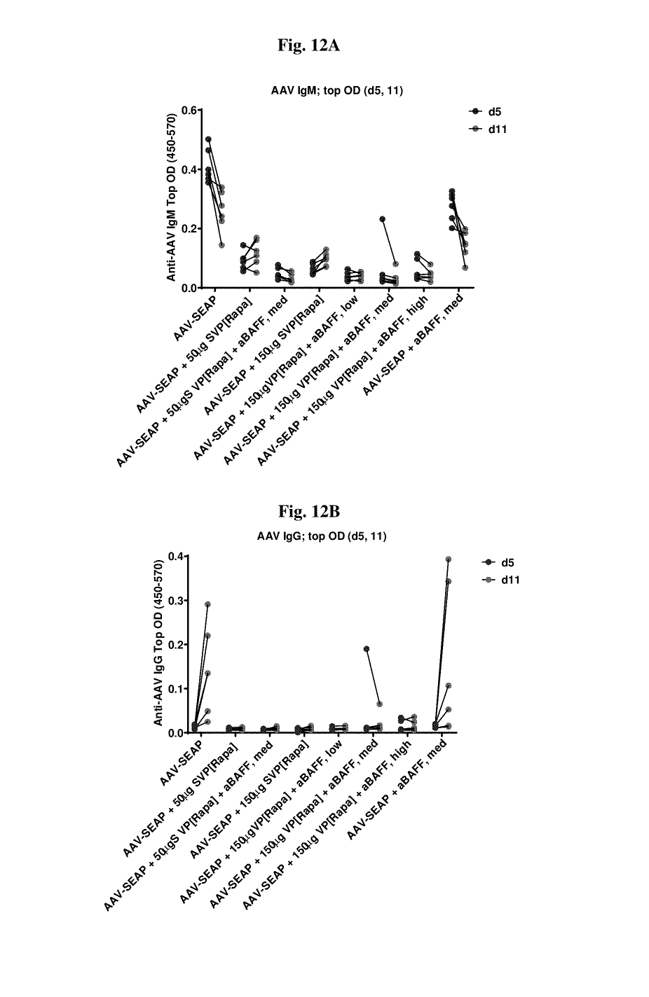

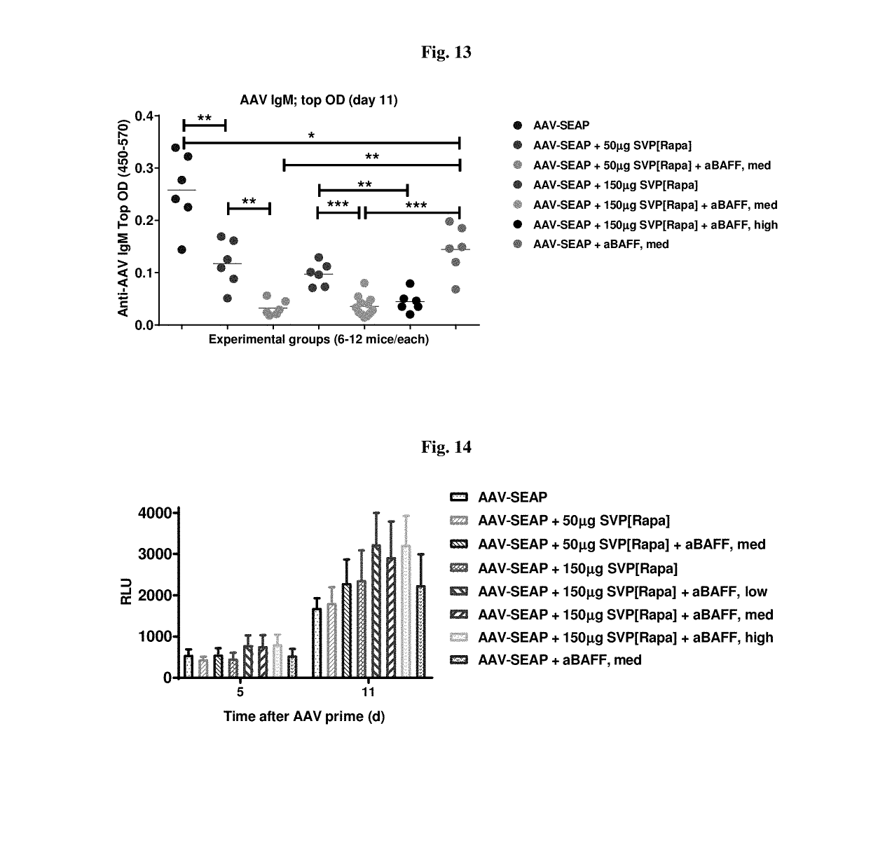

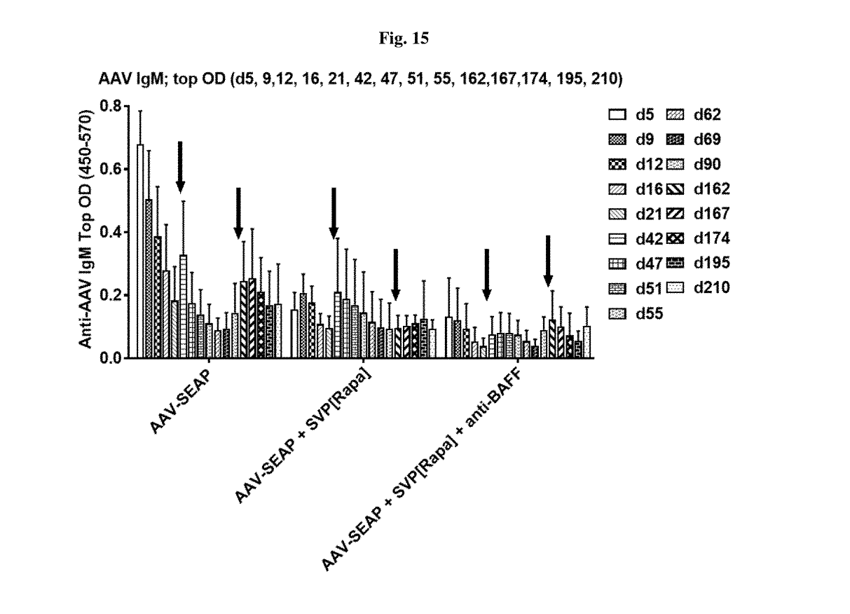

Serum from the mice described above was also analyzed to determine SEAP expression level. As shown in FIG. 2, on days 5, 9, 12, and 16, the administration of AAV-SEAP in combination with synthetic nanocarriers comprising rapamycin and the anti-BAFF antibody yielded greater expression levels of SEAP compared to the two other groups. Additionally, the magnitude of SEAP expression was enhanced at each time point, indicating that the combination leads to improved target transgene expression both initially and over time.

### Example 3: A Synergistic Decrease of In Vivo IgM Immune Response to AAV by Combination of Synthetic Nanocarrier-Encapsulated Rapamycin and Systemic Anti-BAFF

Three groups of C57BL/6 female mice (6 mice each) were injected (i.v., tail vein) three times on days 0, 37 and 155 with 1×10¹⁰ VG of AAV8-SEAP without any nanocarriers (one group) or with SVP[Rapa] at 150 μg (two groups). Of the latter two, one group was additionally treated with systemic anti-BAFF (i.p. 100 μg) (clone Sandy-2 from Adipogen Corp., San Diego, Calif., USA) on days 0, 15, 37, 155 and 169, i.e. at every AAV8 injection and also 14 days after prime and the 2^{nd} boost.

At time indicated (days 5, 9, 12, 16, 21, 42, 47, 51, 55, 162,167,174, 195 and 210) mice were bled, serum separated from whole blood and stored at −20±5° C. until analysis. Then IgM antibodies to AAV was measured in ELISA: 96-well plates coated o/n with the AAV, washed and blocked on the following day, then diluted serum samples (1:40) added to the plate and incubated; plates washed, donkey anti-mouse IgM specific-HRP added and after another incubation and wash, the presence of IgM antibodies to AAV detected by adding TMB substrate and measuring at an absorbance of 450 nm with a reference wavelength of 570 nm (the intensity of the signal presented as top optical density, OD, is directly proportional to the quantity of IgM antibody in the sample).

As is shown in FIG. 15, SVP[Rapa] co-administered with AAV suppressed early induction of AAV IgM and delayed its appearance, especially after prime. However, this was less noticeable after boosts (indicated by arrows), especially after the first of them on d37, resulting in noticeable IgM elevation in the group treated only with SVP[Rapa] during d42-55 interval. At the same time, IgM production in the group treated with SVP[Rapa] and systemic anti-BAFF showed even stronger and statistically more pronounced suppression of IgM response, which was lower than in the group treated only with SVP[Rapa] after first two injections (d0 and 37) and did not statistically exceed it after the 3^{rd} one (d155).

### Example 4: Lower Levels of AAV IgG Breakthroughs are Induced by Combination of Nanocarrier-Encapsulated Rapamycin and Systemic Anti-BAFF

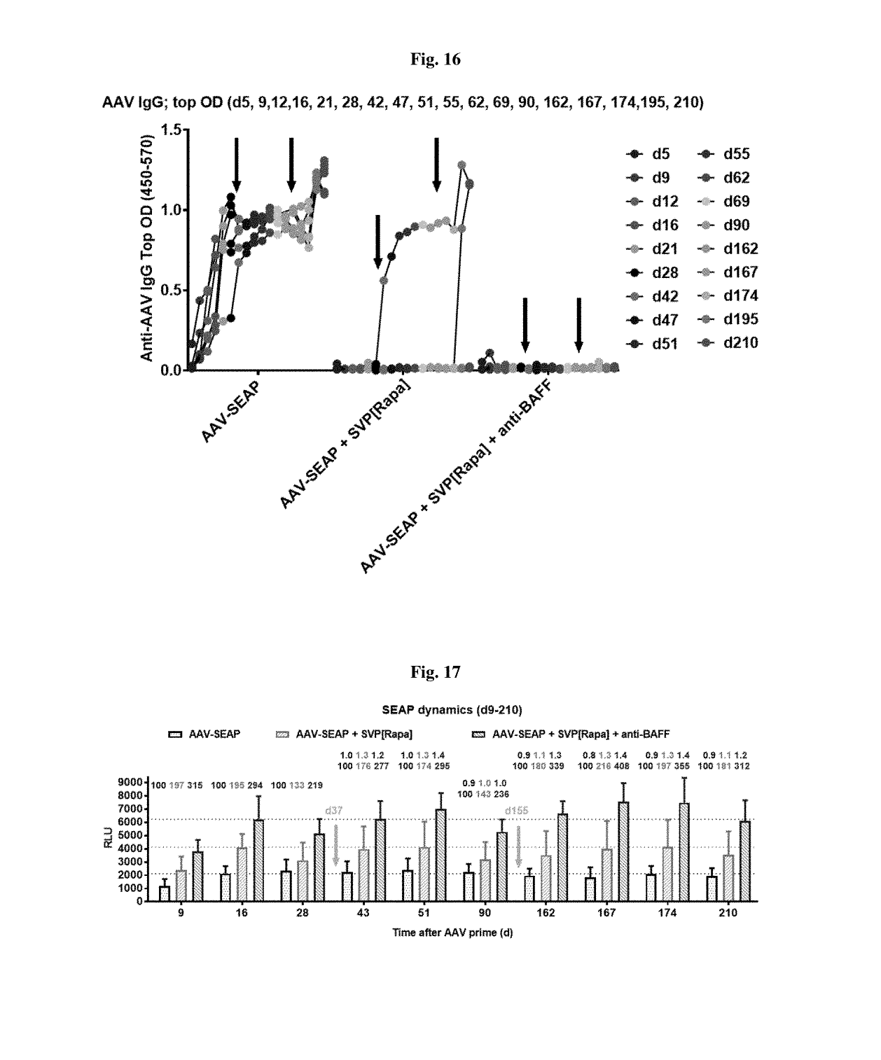

Same serum samples from Example 3 were also tested for AAV IgG measured by ELISA along the same lines as IgM with the exception of goat anti-mouse IgG specific-HRP being used. As has been shown earlier, FIG. 16 shows SVP[Rapa] co-administered with AAV suppressed induction of AAV IgG in the majority of experimental animals, although a few of them started to develop IgG later in the experiment (which correlates with delayed IgM kinetics in this group). Notably, there were no IgG breakthroughs in the group treated with combination of SVP[Rapa] and anti-BAFF, which also correlated with an even lower levels of IgM and more pronounced delay in its production.

### Example 5: A Synergistic Long-Term Enhancement of AAV-Driven Transgene Expression In Vivo by Combination of Nanocarrier-Encapsulated Rapamycin and Systemic Anti-BAFF is Seen after Each AAV Re-Administration

In the same study as Examples 3 and 4, SEAP levels in serum were measured using an assay kit from ThermoFisher Scientific (Waltham, Mass., USA). Sera samples and positive controls were diluted in dilution buffer, incubated at 65° C. for 30 minutes, then cooled to room temperature, plated into 96-well format, assay buffer (5 minutes) and then substrate (20 minutes) added and plates read on luminometer (477 nm).

As is shown in FIG. 17, there was an immediate increase in transgene expression in groups treated with SVP[Rapa]. Of these, serum SEAP elevation in group treated with combination of SVP[Rapa] and anti-BAFF was higher and statistically different from levels generated by treatment with SVP[Rapa] only (relative expression levels for each time point are shown within the graph calculated against levels in untreated group, which were assigned a score of one hundred, 100). Moreover, at every subsequent AAV administration (d37 and 155, shown by arrows in FIG. 17), group administered SVP[Rapa] and anti-BAFF combination showed a further boost in SEAP expression, which was never inferior to one seen in group treated only with SVP[Rapa] and mostly was higher, especially after the 2^{nd} boost (as described earlier, there was no boost in untreated mice; post-to-pre-boost expression levels are shown for all post-boost time points in the top line above the relative expression levels). This resulted in stable and the highest levels of SEAP expression seen in the study. Note that on multiple occasions over more than half a year of the study duration SEAP expression in group treated with SVP[Rapa] and anti-BAFF combination exceed levels seen early on day 16, while these were never exceeded neither in group treated only with SVP[Rapa] or left untreated. Collectively, at multiple time-points SEAP expression levels in group treated with SVP[Rapa] and anti-BAFF combination 3-fold or higher than in group that was treated with AAV only.

### Example 6: A Synergistic Increase of AAV-Driven Transgene Expression and Decrease of IgM and IgG Immune Response to AAV by Combination of Nanocarrier-Encapsulated Rapamycin and Systemic Anti-BAFF is not Seen if Anti-BAFF is Used Alone, without SVP[Rapa]

Four groups of C57BL/6 female mice (6 mice each) were injected (i.v., tail vein) three times on days 0, 32 and 98 with 1×10¹⁰ VG of AAV8-SEAP without any nanocarriers (two groups) or with SVP[Rapa] at 150 μg (two groups). In both arms, one group was left without any additional intervention (i.e., one was completely untreated and one was treated with SVP[Rapa] only) and the other one was additionally treated with systemic anti-BAFF (i.p. 100 μg) on the days of AAV administration (d0, 32, and 98).

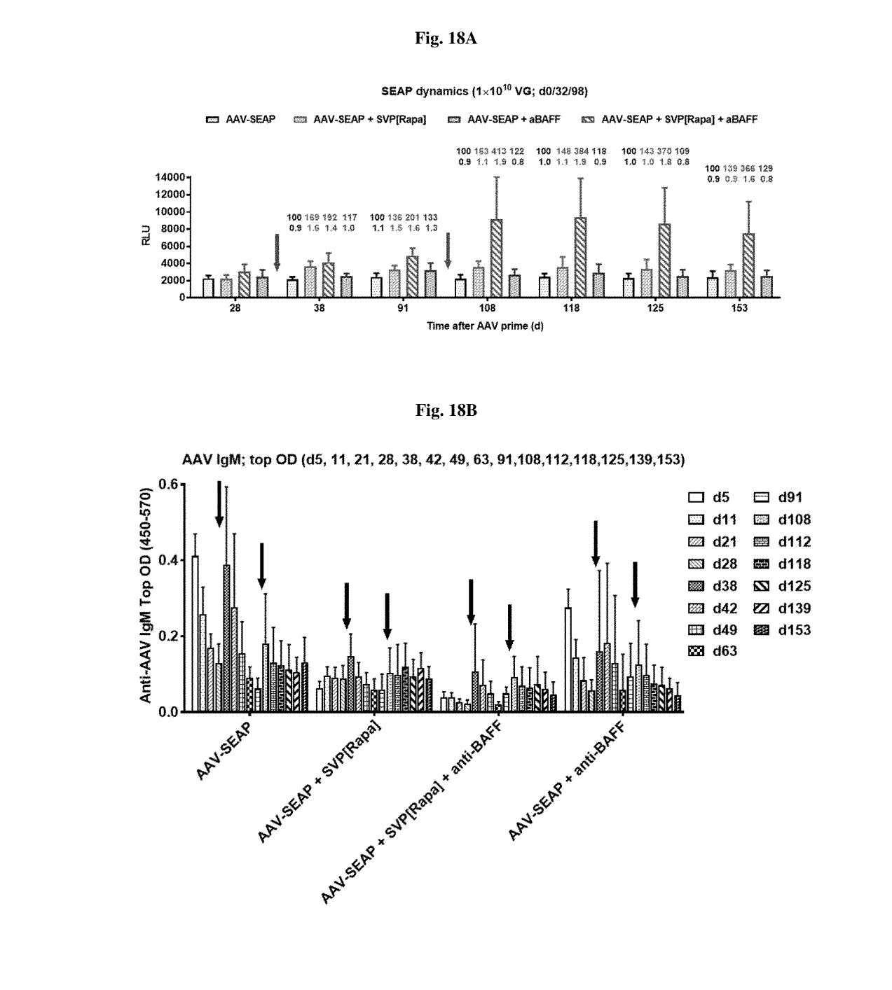

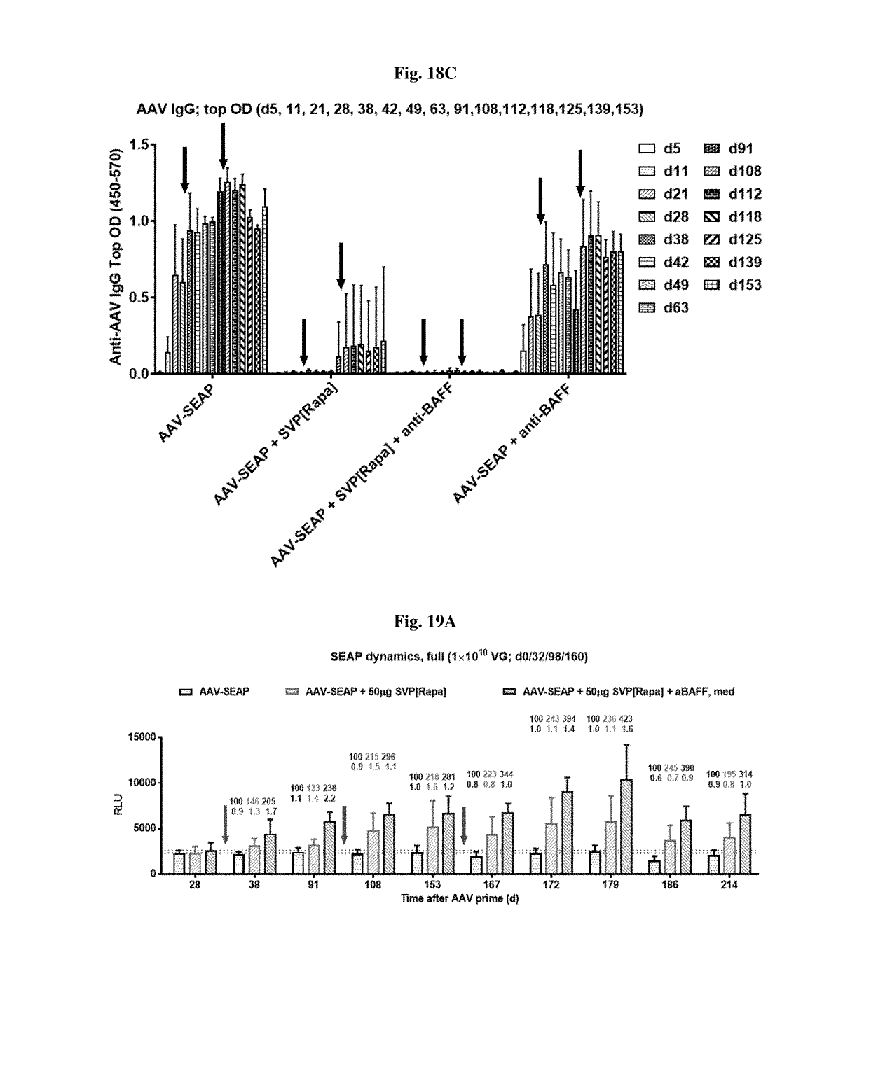

At times indicated (days 5, 11, 21, 28, 38, 42, 49, 63, 91, 108, 112, 118, 125, 139 and 153) mice were bled, serum separated from whole blood and used for determination of SEAP levels (FIG. 18A) as well as IgM and IgG antibodies to AAV as described above (FIGS. 18B-18C).

As is shown in FIG. 18A, while SVP[Rapa] alone provided a certain benefit for transgene expression, there was much higher and statistically different increase of SEAP activity in the group treated with combination of SVP[Rapa] and anti-BAFF, especially after the 2^{nd} boost on day 98 (relative expression levels for each time point are shown calculated against levels in untreated group, assigned a score of ‘100’; post-to-pre-boost expression levels shown for all post-boost time points below the relative expression levels). This collectively resulted in 3.5-4-fold elevation of SEAP expression in the group treated with the combination of SVP[Rapa] and anti-BAFF compared to untreated mice. Importantly, no statistically significant elevation of transgene expression was seen in group treated singly with anti-BAFF, especially after the 2^{nd} boost (the 3^{rd} AAV-SEAP administration).

Conversely, the lowest levels of AAV IgM (and no IgG breakthroughs) were seen in the group treated with the combination of SVP[Rapa] and anti-BAFF compared to other groups. IgM response in this group was especially low after the 1^{st} and 3^{rd} AAV administrations and at multiple time-points was statistically different from all other groups, including that treated only with SVP[Rapa] (FIG. 18B).

While IgM levels were initially slightly delayed and decreased in the group treated only with anti-BAFF, they were always higher than in both groups treated with SVP[Rapa], especially the one treated with the combination of SVP[Rapa] and anti-BAFF (FIG. 18B). Similarly, IgG kinetics was only marginally delayed in this group with majority of mice becoming seropositive by day 21 and all of them converting by day 38 (untreated mice completely converted by day 21), while no mouse in groups treated with SVP[Rapa] has converted up to day 91 and no mouse in group treated with the combination of SVP[Rapa] and anti-BAFF became IgG-positive for the duration of the study (FIG. 18C).

Collectively, while SVP[Rapa] alone showed a benefit for AAV-driven transgene expression and IgM/IgG suppression and anti-BAFF alone demonstrated a certain ability to delay generation of AAV-specific IgM and IgG, the combination of both treatments was far superior in elevating SEAP expression as well as in AAV-specific IgM/IgG suppression, especially after repeated AAV administrations.

### Example 7: A Synergistic Increase of AAV-Driven Transgene Expression Coupled with Continued Suppression of IgM and IgG Immune Response to AAV by Combination of Nanocarrier-Encapsulated Rapamycin and Systemic Anti-BAFF is Seen after Multiple AAV Administrations

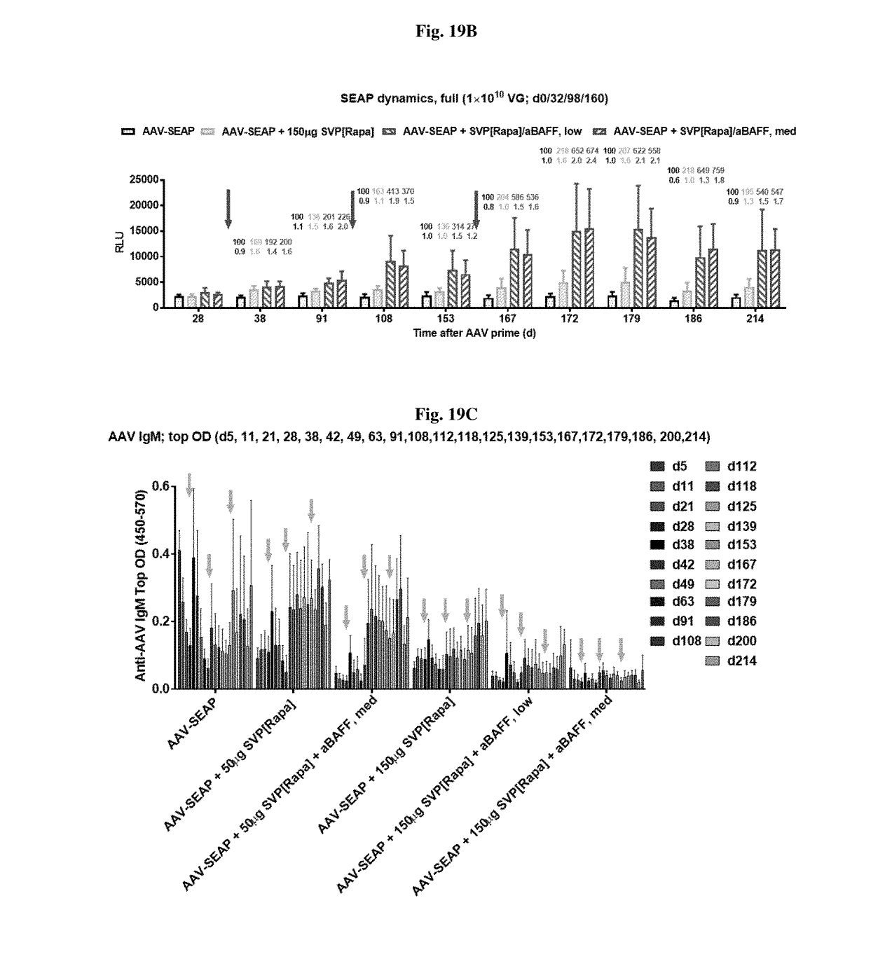

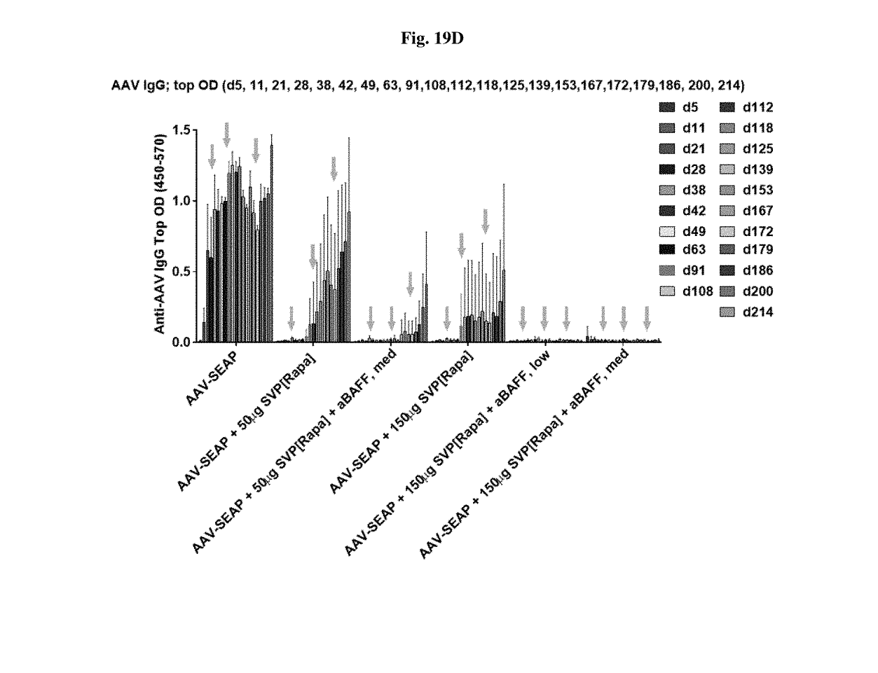

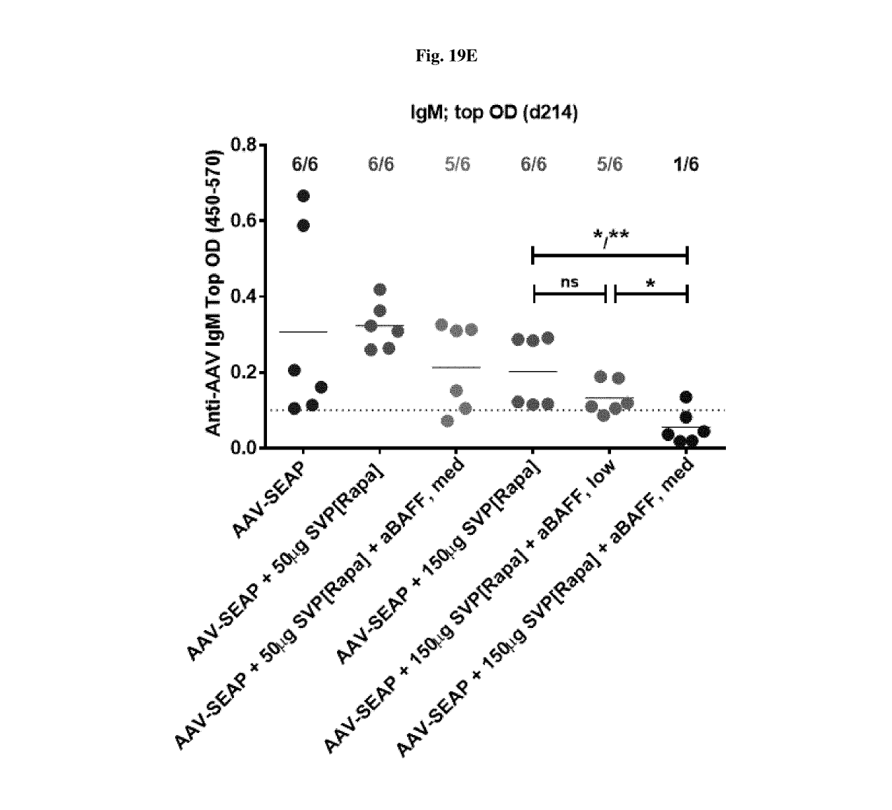

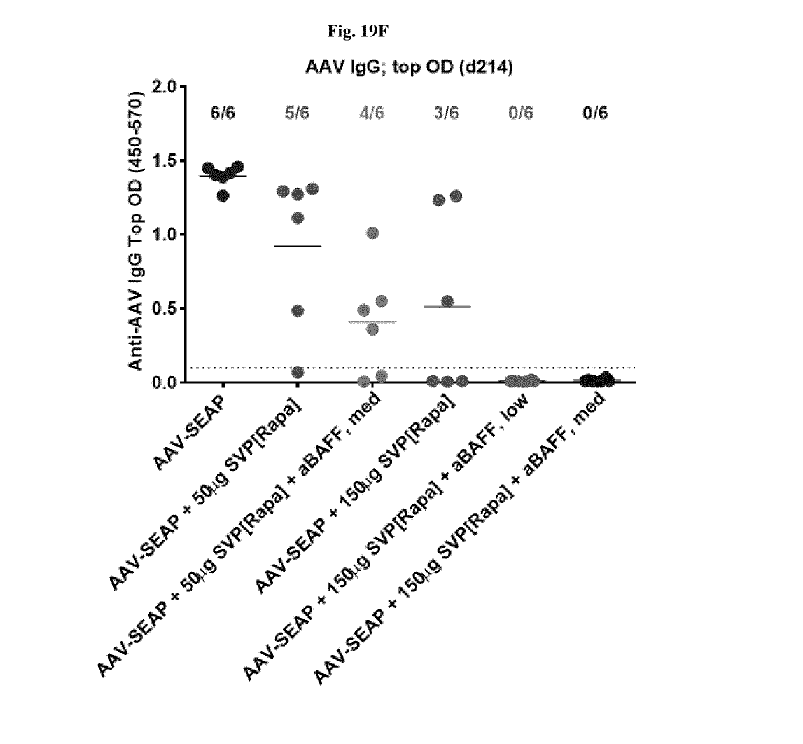

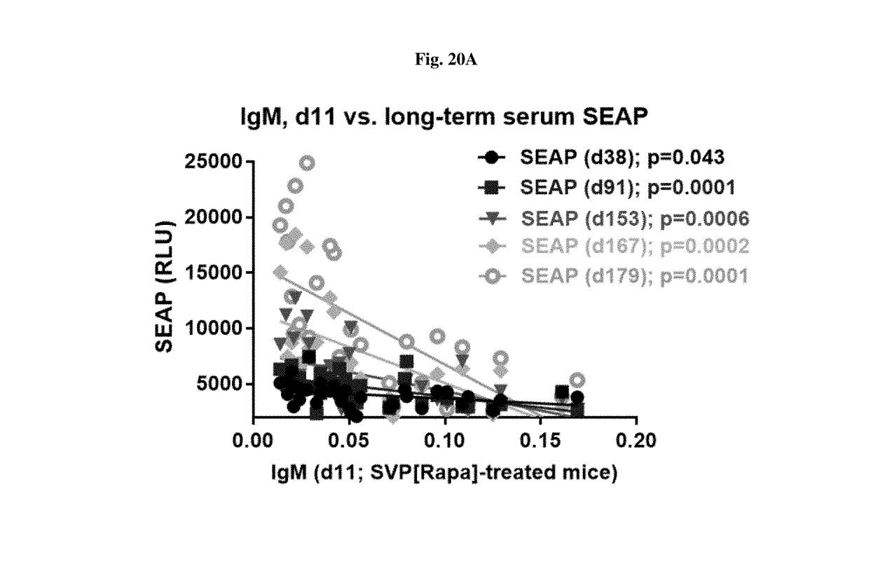

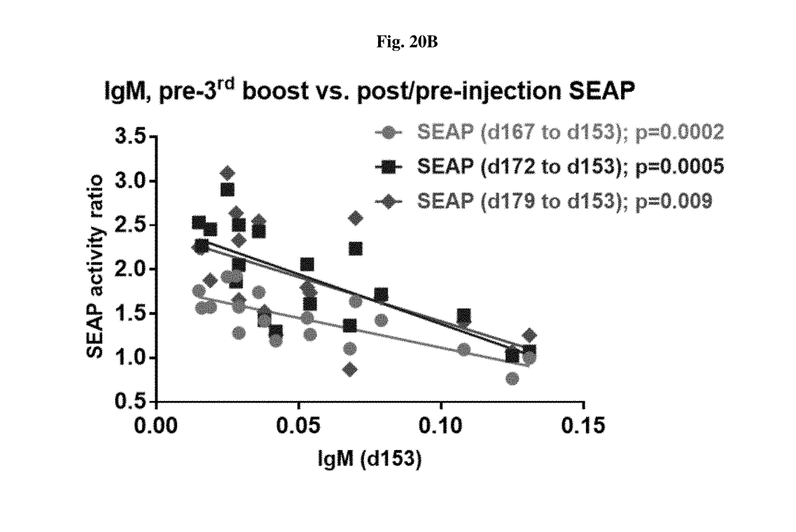

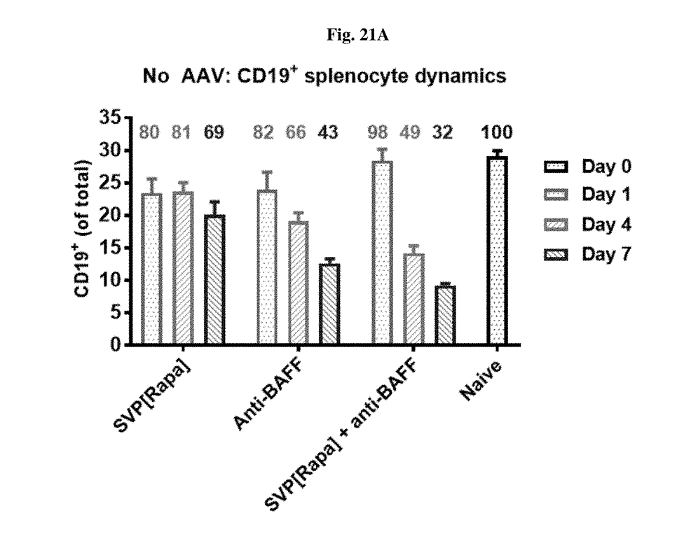

Six groups of C57BL/6 female mice (6 mice each) were injected (i.v., tail vein) four times on days 0, 32, 98, and 160 with 1×10¹⁰ VG of AAV8-SEAP either alone or combined with different doses of SVP[Rapa] (50 or 150 μg) with or without additional treatment with systemic anti-BAFF (i.p., 100 μg), administered either only on injection day, thus equalling four treatments total and defined as ‘low’ or also given at 14 days after the 1^{st}, the 3^{rd} and the 4^{th} AAV administrations, i.e., days 14, 112 and 174 of the study thus equalling seven total treatments and defined as ‘medium’. At times indicated (days 28, 38, 91, 108, 153, 167, 172, 179, 186 and 214) mice were bled, serum separated from whole blood and used for determination of SEAP levels (FIGS. 19A-19B) as well as IgM and IgG antibodies to AAV as described above (FIGS. 19C-19F).

Notably, at both SVP[Rapa] doses, administering anti-BAFF provided a significant late boost in SEAP expression, which was well-manifested after the last AAV injection at day 160 with anti-BAFF and 50 μg SVP[Rapa] combination showing considerable elevation for nearly three weeks post injection (FIG. 19A) and the same combination with 150 μg SVP[Rapa] demonstration continuous transgene elevation up to 8 weeks post injection (FIG. 19B), which in both cases was much more pronounced and statistically different from benefit attained by SVP[Rapa] used alone (relative expression levels for each time point are shown calculated against levels in untreated group, assigned a score of ‘100’; post-to-pre-boost expression levels shown for all post-boost time points below the relative expression levels). At every subsequent injection groups treated with SVP[Rapa] and, more so, with SVP[Rapa] and anti-BAFF combination showed an increase in transgene activity, while untreated mice did not (see day 28 SEAP activity levels for each group marked by dotted lines in FIG. 19A) and thus collectively at several time-points the cumulative effect of SVP[Rapa] and anti-BAFF was close or more than 7-fold compared to the group injected 4 times with AAV-SEAP without any additional treatment (FIG. 19B).

Both IgM and IgG to AAV continued to be profoundly suppressed for the duration of the study with IgM to AAV especially well suppressed in the group treated with combination of 150 μg SVP[Rapa] and medium anti-BAFF (FIG. 19C and FIG. 19E). IgM response in this group stayed at the baseline in the majority of mice till day 214 of the study (FIG. 19E), becoming statistically different from all other groups (number of IgM and IgG breakthroughs in each group, defined as top OD of >0.1 is shown in FIG. 19C and FIG. 19D). Both groups treated with 150 μg SVP[Rapa] combined with anti-BAFF showed no IgG breakthroughs till the end of the study (FIG. 19D and FIG. 19F)

### Example 8: Early and Late IgM Levels in Mice Administered SVP[Rapa] with or without Anti-BAFF Inversely Correlate with a Long-Term Expression of AAV-Driven Transgene

Five groups of C57BL/6 female mice (6 mice each) were injected (i.v., tail vein) four times on days 0, 32, 98, and 160 with 1×10¹⁰ VG of AAV8-SEAP combined with different doses of SVP[Rapa] (50 or 150 μg) with or without additional treatment with systemic anti-BAFF (i.p., 100 μg). As is shown in FIG. 20, all of these mice demonstrated a delay in forming AAV IgM, which was markedly suppressed at day 11 (mice non-treated with SVP[Rapa] are uniformly IgM-positive by day 5, see earlier examples), although a few mice have seroconverted by that time. When day 11 IgM values were plotted against serum SEAP levels determined prior to and after each of three subsequent AAV boosts administered on days 32, 98 and 160, all of these datasets showed a statistically significant inverse correlation, which strengthened with time (from p=0.043 on day 38 to p=0.0001 on day 179, see FIG. 20A), therefore indicating that early IgM response can be determinative of AAV transduction and subsequent long-term transgene expression.

Similarly, when IgM levels on d153 (one week prior to 4^{th} AAV inoculation=the 3^{rd} boost) seen in mice treated with 150 μg SVP[Rapa] with or without anti-BAFF were plotted against post-boost SEAP elevation (as the ratio of post- to pre-boost expression levels), similarly strong inverse correlation was seen (FIG. 20B).

Collectively, this indicates that both early and long-term IgM responses to AAV can be determinative of AAV-driven transgene expression levels, especially after repeated AAV administrations and that antigen-specific IgM suppression as attained by the combination of SVP[Rapa] and anti-BAFF can be beneficial and can result in long-term and stable transgene expression in vivo.

### Example 9: Combination of SVP[Rapa] with Anti-BAFF Decreases Suppresses General and Specific Splenic B Cell Populations in Naïve and AAV-Injected Mice

Seven groups of C57BL/6 female mice (9 mice each, 3 mice per each time-point) were either injected (i.v., tail vein) with 1×10¹⁰ VG of AAV8-SEAP (four groups) or left virus-naive (three groups). Of the former, one group received no further treatment, one was co-injected with 150 μg of SVP[Rapa], one was additionally treated with anti-BAFF (i.p., 100 μg) and the last one was treated with combination of SVP[Rapa] and systemic anti-BAFF. Similarly, three groups not injected with AAV, were treated with 150 μg of SVP[Rapa], anti-BAFF (i.p., 100 μg) and with their combination. Mice receiving no injection served as baseline control (day 0).

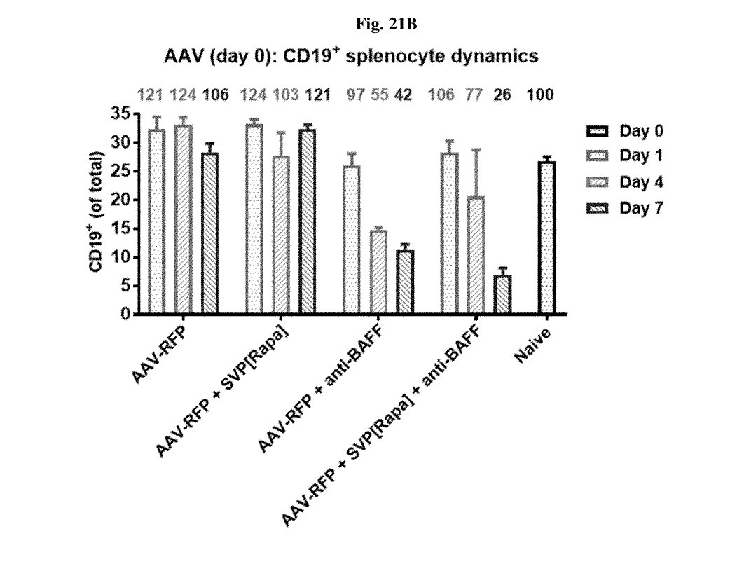

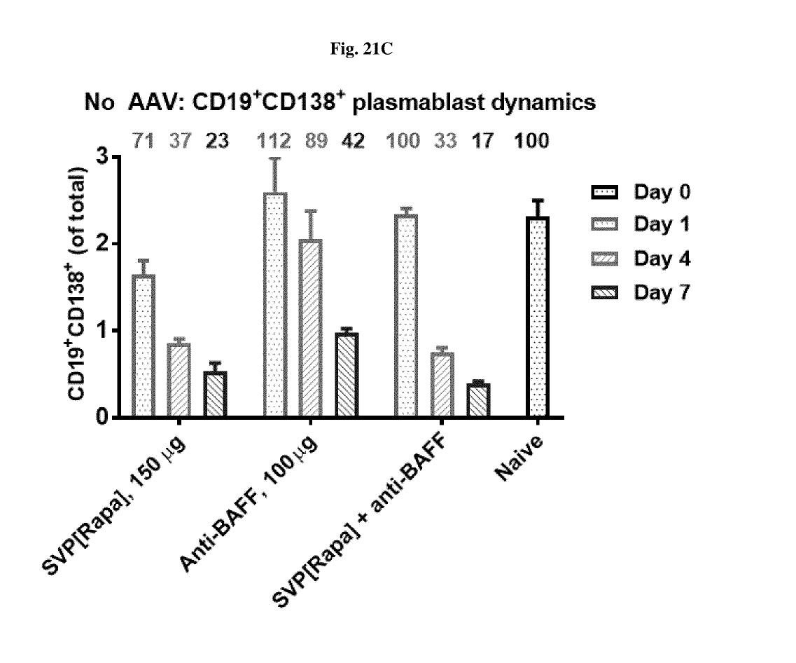

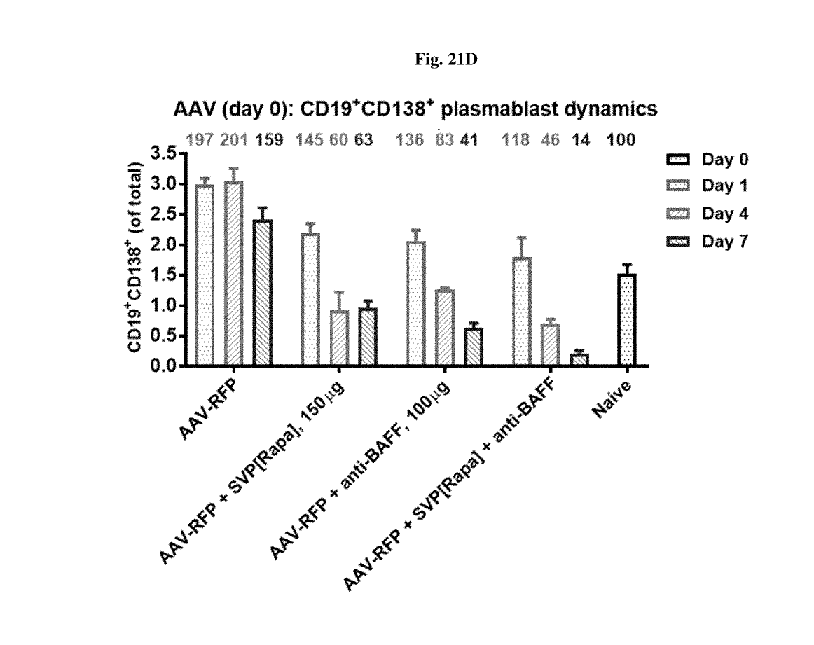

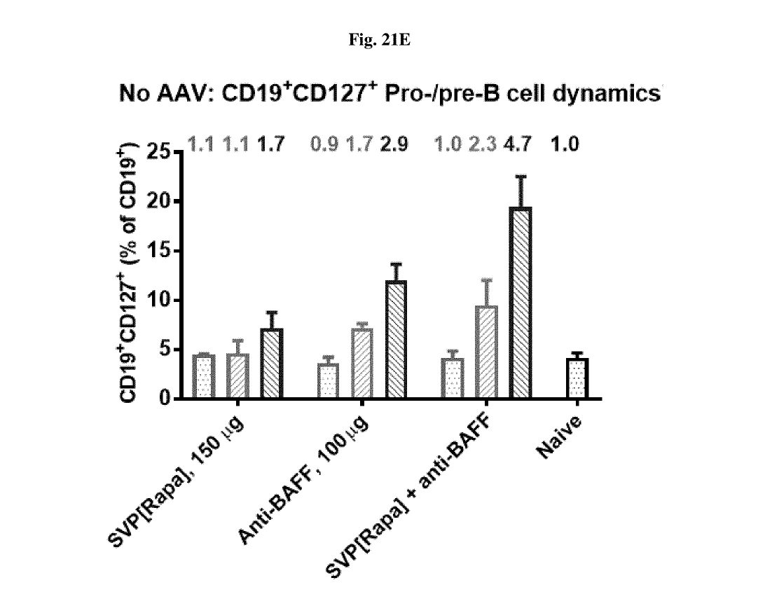

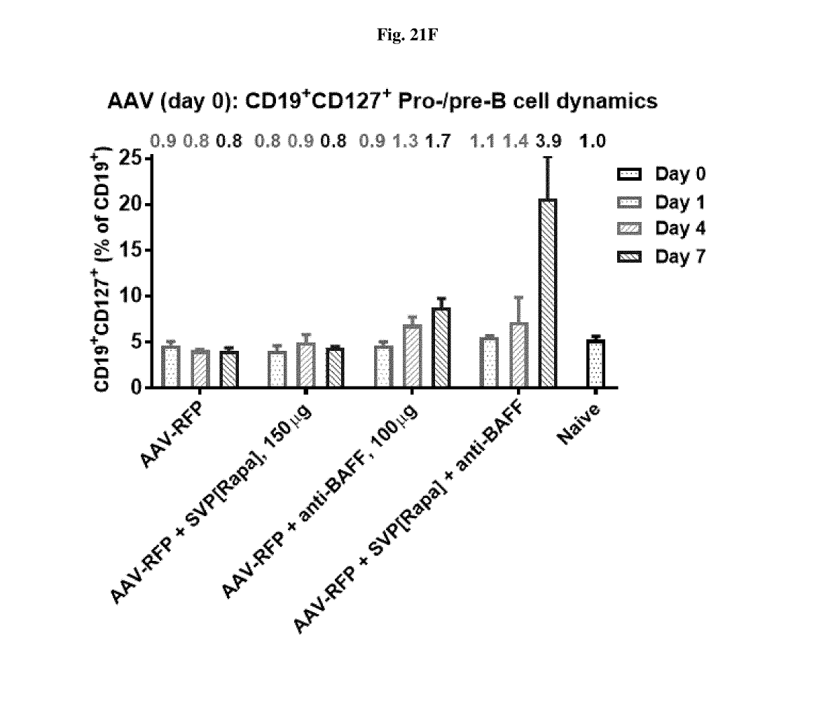

At times indicated (1,4 and 7 days after injection) mice were sacrificed, spleens taken, meshed to single cell suspensions and then stained with antibodies to B cell surface markers CD19, CD138, and CD127. As seen in graphs FIG. 21A and FIG. 21B, AAV-injected mice, untreated or treated with SVP[Rapa], did not experience any decrease in total number of splenocytes of B cell origin (defined as CD19⁺). Similarly, virus-naïve mice treated with SVP[Rapa] showed only a minor decrease in number of CD19⁺ cells. Conversely, mice treated with anti-BAFF (whether AAV-injected or virus-naïve) showed a profound and time-dependent drop in CD19⁺splenic cells (at least by a factor of 2), which was even more pronounced if SVP[Rapa] was also used (by a factor of 3-4).

This effect was even more salient if the fraction of plasmablast cells (defined as CD19⁺CD138⁺), direct precursors of antibody-secreting long-lived plasma cells was evaluated (FIG. 21C and FIG. 21D). In this case, SVP[Rapa] treatment led to time-dependent splenic plasmablast decrease as did anti-BAFF treatment (by a factor of 2-3; there were virtually no changes in untreated AAV-injected mice). However, cumulative effect of combination treatment with SVP[Rapa] and anti-BAFF was even stronger resulting in more than 7-fold decrease in plasmablast fraction, showing that this combination can act specifically against antibody-producing cells of B cell lineage.

This was reciprocally reflected in relative increase of pre-/pro-B cell fraction (i.e., immediate precursors of immature B cells, defined as CD19⁺CD127⁺) as shown in graphs FIG. 21E and FIG. 21F. In this case, untreated and SVP[Rapa]-treated AAV-injected mice showed no changes in pre-/pro-B cell dynamics and the effect of SVP[Rapa] on virus-naïve mice was less than 2-fold and seen only by day 7. Anti-BAFF exhibited a stronger effect, which was seen both in virus-naïve and AAV-injected mice, being noticeably less profound in the former. Notably, the combination treatment with SVP[Rapa] and anti-BAFF again exhibited a synergistic effect (being higher than arithmetic sum of effects of single treatments with SVP[Rapa] and anti-BAFF), elevating the fraction of immature B cell precursors nearly 4-fold in AAV-injected mice and even higher in virus-naïve ones. Collectively, it appeared that combination treatment with SVP[Rapa] and anti-BAFF led to specific and early block in B cell maturation both in virus-naïve mice and, more importantly, even in case of AAV infection, which correlated with a profound suppression of virus-specific IgM and IgG production accomplished by this combination treatment.

### Example 10: A Synergistic Decrease of In Vivo IgM Immune Response to AAV by Combination of Nanocarrier-Encapsulated Rapamycin and Systemic Administration of Bruton Tyrosine Kinase Inhibitor PCI-32765 (Ibrutinib)

Five groups of C57BL/6 female mice (6 mice each) were injected (i.v., tail vein) twice on days 0 and 93 with 1×10¹⁰ VG of AAV8-SEAP without any nanocarriers (one group) or with SVP[Rapa] at 100 μg (four groups). Of the latter, three groups were treated with systemic ibrutinib (i.p. 200 μL) for 17 consecutive days daily at the following doses: 20, 100 or 500 μg/mouse starting with 2 days prior to AAV-SEAP and SVP[Rapa] injection (days −2 to 14 and days 91 to 107).

At time indicated (days 6, 9, 14, 21, 28, 49, 63, 91, 97, 100, 104, and 111) mice were bled, serum separated from whole blood and stored at −20±5° C. until analysis. Then IgM antibody to AAV was measured in ELISA: 96-well plates coated o/n with the AAV, washed and blocked on the following day, then diluted serum samples (1:40) added to the plate and incubated; plates washed, donkey anti-mouse IgM specific-HRP added and after another incubation and wash, the presence of IgM antibodies to AAV detected by adding TMB substrate and measuring at an absorbance of 450 nm with a reference wavelength of 570 nm (the intensity of the signal presented as top optical density, OD, is directly proportional to the quantity of IgM antibody in the sample).

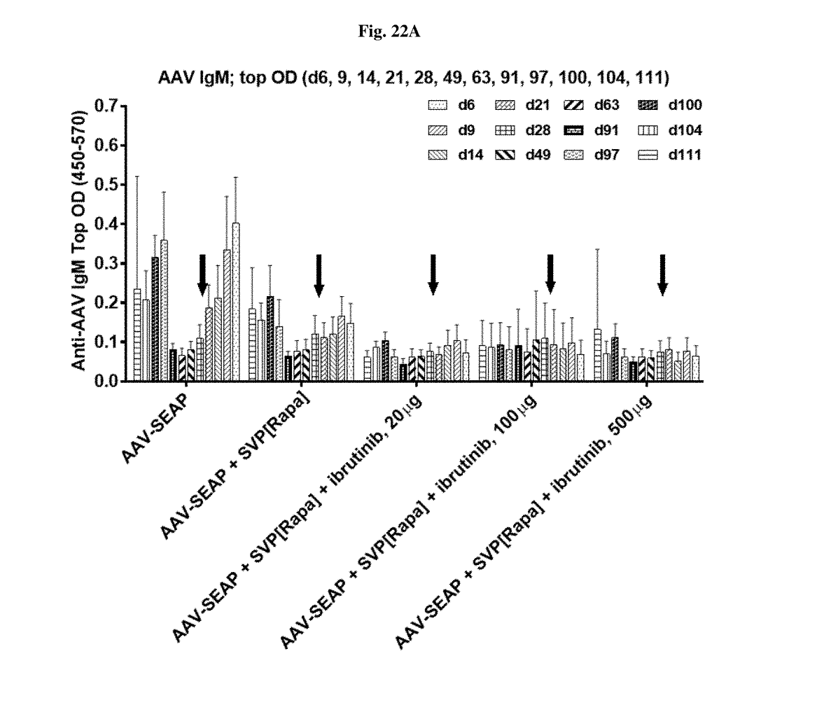

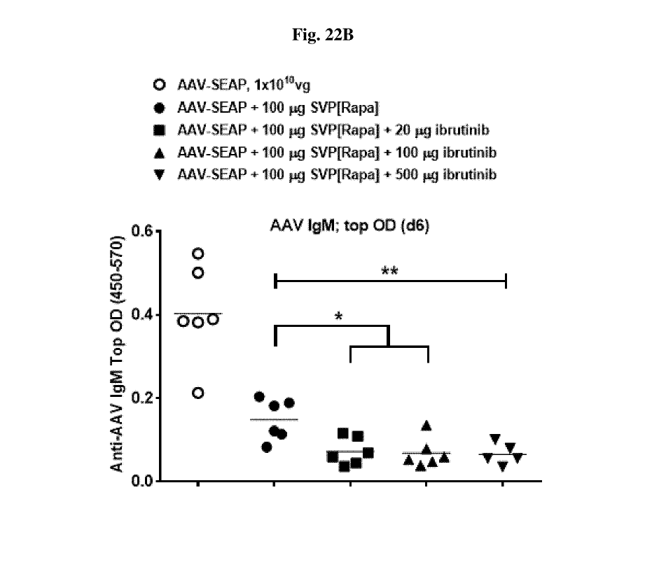

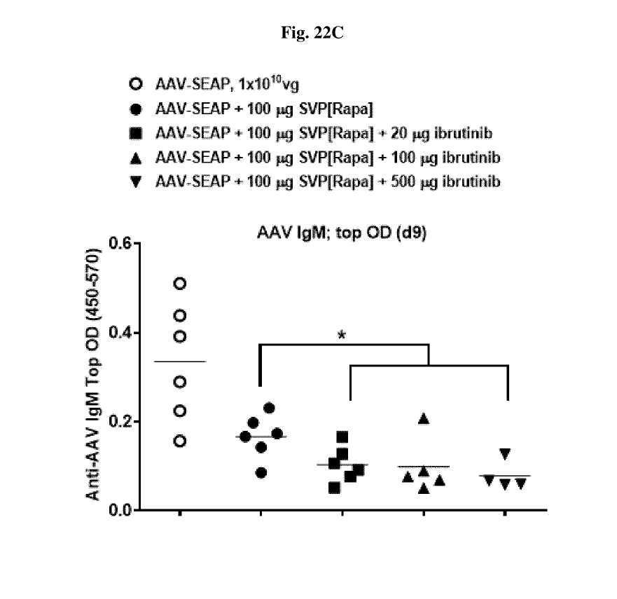

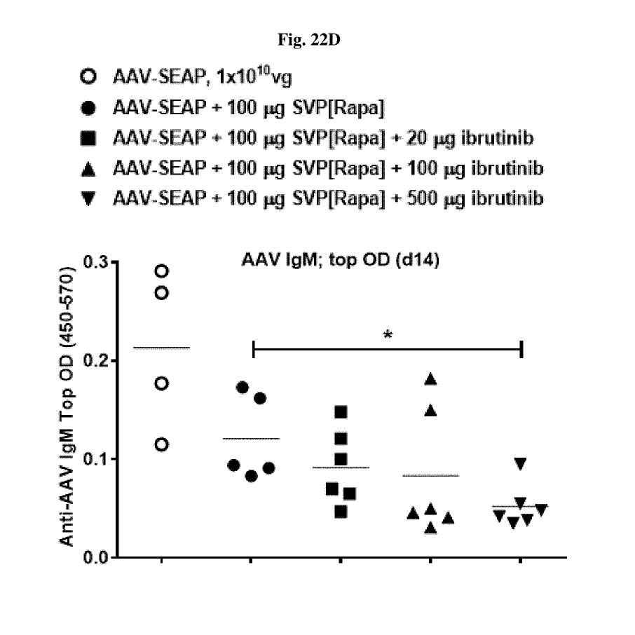

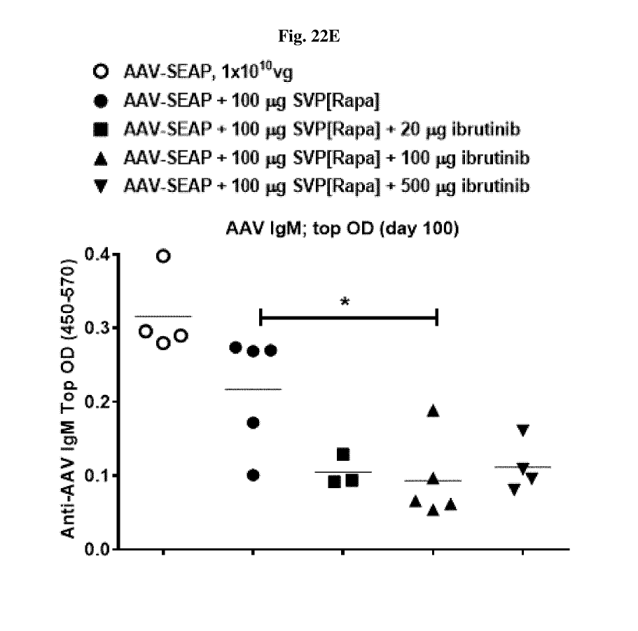

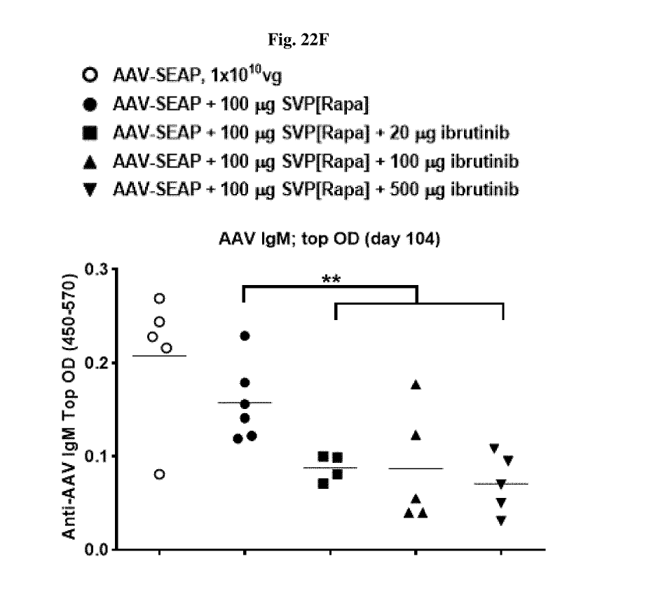

As is shown in FIG. 22, SVP[Rapa] co-administered with AAV suppressed early induction of AAV IgM and delayed its appearance (FIG. 22A). However, in the group treated only with SVP[Rapa] IgM was generally detectable and also demonstrated a certain boost after repeat AAV injection at d93 (shown by an arrow in FIG. 22A). At the same time, all the groups co-treated with SVP[Rapa] and systemic ibrutinib showed even stronger and statistically more pronounced suppression of early IgM response, which at the high ibrutinib dose of 500 μg was statistically different from the group treated only with SVP[Rapa] up to day 14 (FIGS. 22B-22D). Furthermore, all of the groups treated with combination of SVP[Rapa] and systemic ibrutinib showed statistically lower IgM levels compared to group treated only with SVP[Rapa] soon after day 93 repeat AAV injection (FIGS. 22E-22F).

### Example 11: A Synergistic Post-Boost Enhancement of AAV-Driven Transgene Expression In Vivo by Combination of Nanocarrier-Encapsulated Rapamycin and Systemic Ibrutinib Inversely Correlating with Early AAV IgM

In the same study as Example 10, SEAP levels in serum were measured using an assay kit from ThermoFisher Scientific (Waltham, Mass., USA) as described above: samples diluted in dilution buffer, incubated at 65° C. for 30 minutes, then cooled to room temperature, plated into 96-well format, assay buffer (5 minutes) and then substrate (20 minutes) added and plates read on luminometer (477 nm).

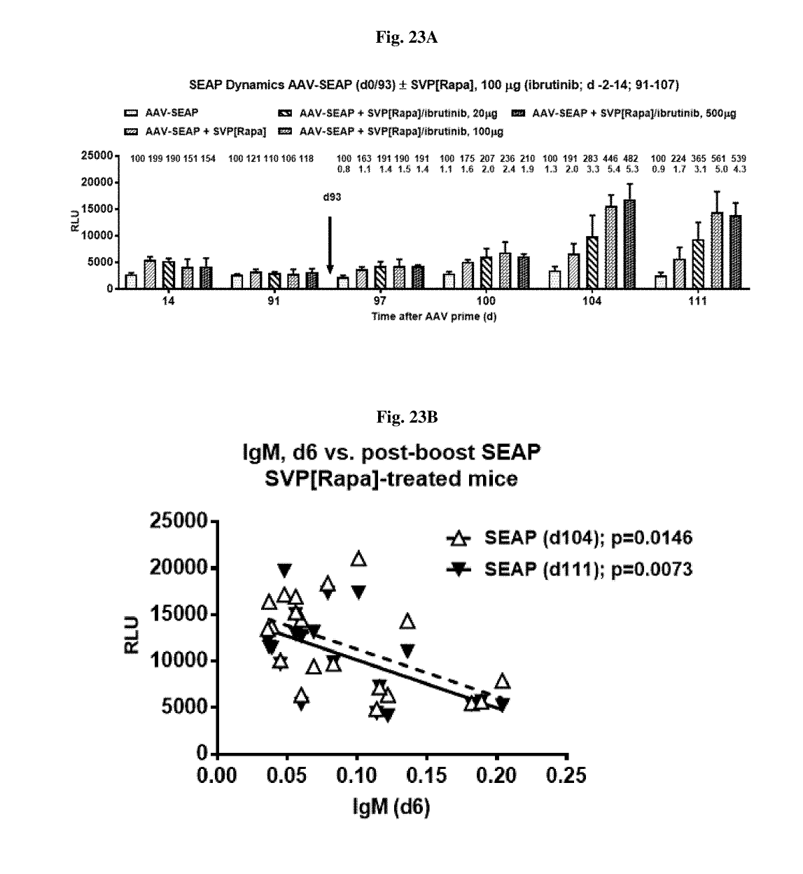

There was no noticeable difference in initial SEAP expression levels among all the groups treated with SVP[Rapa] irrespective of ibrutinib administration, although all of these showed higher levels of serum SEAP compared to the group not treated with SVP[Rapa] (see day 14 data in FIG. 23A; SEAP levels in mice receiving AAV-SEAP without any other treatments are assigned a number of ‘100’ at all time-points and the relative expression in all other groups calculated accordingly). When measured at a later time-point (day 91, i.e. two days before the repeat AAV administration; FIG. 23A), all the test groups showed approximately the same level of SEAP expression.

Immediately after the repeat AAV-SEAP administration at day 93, all the groups treated with SVP[Rapa] showed an elevation of transgene expression (FIG. 23A). While group of mice treated only with SVP[Rapa] had SEAP levels exceeding those in untreated mice by 63-75% (FIG. 23A, days 97-100, i.e. 4-7 days after the boost), a higher elevation was seen in all mice treated with combination of SVP[Rapa] and free ibrutinib (more than 2-fold compared to untreated mice at day 100), although at that point the effect seen was not dependent on ibrutinib dose. This started to change by day 104 (11 days after AAV boost) with groups of mice treated with SVP[Rapa] and ibrutinib combination continuing to exhibit elevated SEAP levels exceeding 5-fold difference vs. untreated mice (for the highest ibrutinib doses of 100 and 500 μg) and being more than two times higher than that in mice treated only with SVP[Rapa] (FIG. 23A). There seemed to be a dose-dependency in this example seen starting from day 104 with the highest expression levels seen in mice treated with SVP[Rapa] combined with 100-500 μg of ibrutinib compared to the group, in which 20 μg ibrutinib was used. Notably, early (day 6 post prime) levels of AAV IgM in SVP[Rapa]-treated mice inversely correlated with post-boost serum SEAP levels (FIG. 23B), suggesting that early IgM suppression (more pronounced in mice treated with SVP[Rapa] combined with ibrutinib) can result in lower levels of immune memory to AAV and, as a result, to lower anamnestic responses after repeat AAV administration and a much more sustained and elevated transgene expression post boost.

### Example 12: A Synergistic Decrease of IgM and IgG Immune Response to AAV by Combination of Nanocarrier-Encapsulated Rapamycin and Systemic Ibrutinib is Stronger than that Achieved by Rapamycin or Ibrutinib Used Alone

Four groups of C57BL/6 female mice (8-10 mice each) were injected (i.v., tail vein) three times on days 0, 51 and 105 with 1×10¹⁰ VG of AAV8-SEAP without any nanocarriers (two groups) or with SVP[Rapa] at 100 μg (two groups). In both pairs of groups, one group was additionally treated with systemic ibrutinib (i.p. 500 μg) daily for 17 days starting at 2 days prior to concluding at day 14 after every AAV8 injection (days −2 to 14, days 49 to 65 and days 103 to 119 with AAV-SEAP injection date regarded as day 0 of the experimental timeline).

At time indicated (days 6, 9, 15, 22, 29, 36, 43, 49, 58, 65, 72 and 79) mice were bled, serum separated from whole blood and stored at −20±5° C. until analysis. Then IgM antibodies to AAV were measured in ELISA: 96-well plates coated o/n with the AAV, washed and blocked on the following day, then diluted serum samples (1:40) added to the plate and incubated; plates washed, donkey anti-mouse IgM specific-HRP added and after another incubation and wash, the presence of IgM antibodies to AAV detected by adding TMB substrate and measuring at an absorbance of 450 nm with a reference wavelength of 570 nm (the intensity of the signal presented as top optical density, OD, is directly proportional to the quantity of IgM antibody in the sample).

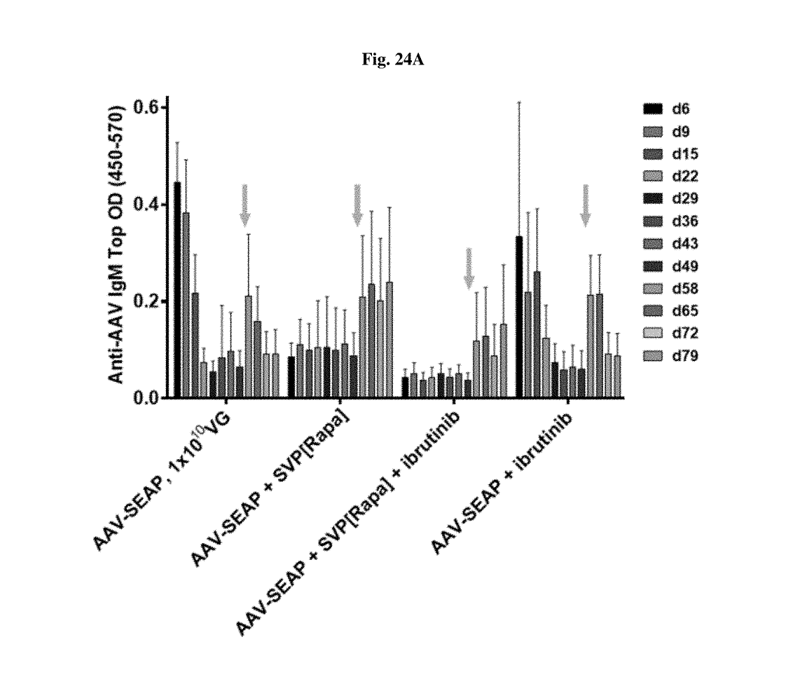

As is shown in FIG. 24, SVP[Rapa] co-administered with AAV suppressed early induction of AAV IgM and delayed its appearance, especially after prime (FIG. 24A, gr. 2). However, this was less noticeable after d51 boost (indicated by arrows), resulting in noticeable IgM elevation in the group treated only with SVP[Rapa] during d58-79 interval. At the same time, IgM production in the group treated with SVP[Rapa] and systemic ibrutinib (FIG. 24A, gr. 3) showed even stronger and statistically more pronounced suppression of IgM response, which was lower than in the group treated only with SVP[Rapa] after first two injections (d0 and 51). Importantly, systemic ibrutinib alone (FIG. 24A, gr. 4) was completely inefficient in IgM suppression showing the same dynamics of its induction as an untreated group 1 (FIG. 24A).

This can be translated to IgG dynamics as well (FIG. 24B) with untreated and ibrutinib-only treated mice (gr. 1 and 4, correspondingly) producing essentially similar and robust response with all animals (8/8 and 10/10) converting by d22, while SVP[Rapa]-treated mice (gr. 2) exhibited delayed and suppressed IgG kinetics with 2/10 of animals converting by d22 and only 4/10 animals showing detectable IgG levels prior to boost (d49). This suppression persisted after d51 boost with only 5/10 animals becoming AAV IgG-positive by d79 (28d post-boost). Still, the combination of SVP[Rapa] and systemic ibrutinib was superior to SVP[Rapa] used alone (and statistically different from it by d79) with no conversions (0/9) immediately prior to boost (d49) and only 1/9 post-boost conversion at d79.

### Example 13: A Synergistic Elevation of Transgene Expression after Repeated AAV Immunizations by Combination of Nanocarrier-Encapsulated Rapamycin and Systemic Ibrutinib is Higher than that Achieved by Rapamycin or Ibrutinib Used Alone

In the same study as Example 12, SEAP levels in serum were measured using an assay kit from ThermoFisher Scientific as described above.

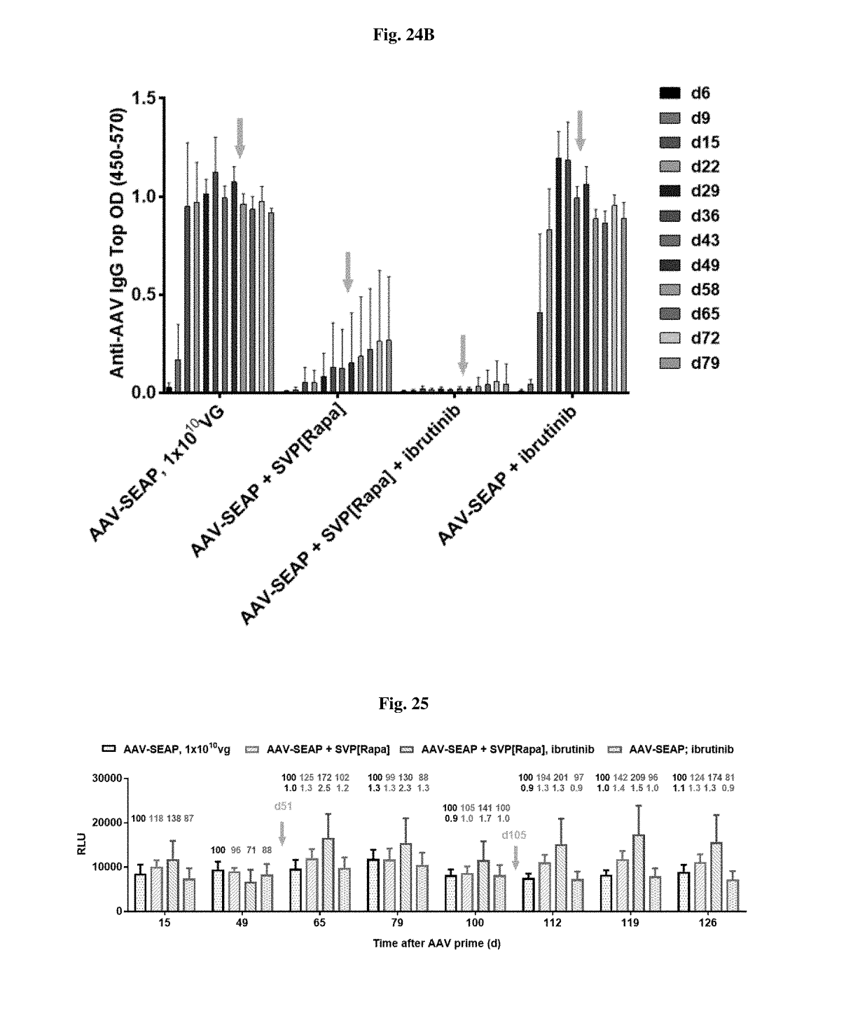

As is shown in FIG. 25, there was an immediate, albeit minor increase in transgene expression in groups treated with SVP[Rapa]. Of these, serum SEAP elevation in group treated with combination of SVP[Rapa] and ibrutinib was higher although not statistically different from levels generated by treatment with SVP[Rapa] only (relative expression levels for each time point are shown within FIG. 25 calculated against levels in untreated group, which were assigned a score of one hundred, 100), while ibrutinib used alone showed no effect vs. untreated mice. Moreover, at every subsequent AAV administration (d51 and 105, shown by arrows), group administered SVP[Rapa] and ibrutinib combination showed the highest boost in SEAP expression, which was never inferior to one seen in group treated only with SVP[Rapa] and mostly was higher, especially after initial boost (post-to-pre-boost expression levels are shown for all post-boost time points in the bottom line below the relative expression levels). As is shown, there was no boost in untreated mice similarly to the group treated with ibrutinib alone. This resulted in stable and the highest levels of SEAP expression seen in the study exhibited in group 3, treated with a combination of SVP[Rapa] and systemic ibrutinib. Collectively, at multiple time-points SEAP expression levels in the AAV-injected group treated with SVP[Rapa] and ibrutinib combination was 2-fold higher than in groups that were treated with AAV only or with AAV+ibrutinib.

### Example 14: AAV Immunizations with Nanocarrier-Encapsulated Rapamycin and Rituximab (Prophetic)

Three groups of C57BL/6 female mice are injected (i.v., tail vein) three times on days 0, 37 and 155 with AAV8-SEAP without any nanocarriers (one group) or with SVP[Rapa] at 150 μg (two groups). Of the latter two, one group is additionally treated with Rituximab on days 0, 15, 37, 155 and 169, i.e. at every AAV injection and also 14 days after prime and the 2^{nd} boost.

At time indicated (days 5, 9, 12, 16, 21, 42, 47, 51, 55, 162,167,174, 195 and 210) mice are bled, and serum is separated from whole blood and stored at −20±5° C. until analysis. Then IgM and IgG antibodies to Ad are measured in ELISA. SEAP levels in serum are measured using an assay kit from ThermoFisher Scientific (Waltham, Mass., USA).

### Example 15: AAV Immunizations with Synthetic Nanocarriers Comprising GSK1059615 and Anti-BAFF Antibody (Prophetic)

Three groups of C57BL/6 female mice are injected (i.v., tail vein) three times on days 0, 37 and 155 with AAV8-SEAP without any nanocarriers (one group) or with Synthetic Nanocarriers Comprising GSK1059615 (two groups). Of the latter two, one group is additionally treated with systemic anti-BAFF (i.p. 100 μg) on days 0, 15, 37, 155 and 169, i.e. at every AAV8 injection and also 14 days after prime and the 2^{nd} boost.

At time indicated (days 5, 9, 12, 16, 21, 42, 47, 51, 55, 162,167,174, 195 and 210) mice are bled, and serum is separated from whole blood and stored at −20±5° C. until analysis. Then IgM and IgG antibodies to Ad are measured in ELISA. SEAP levels in serum are measured using an assay kit from ThermoFisher Scientific (Waltham, Mass., USA).

1. A composition comprising:
a viral transfer vector, synthetic nanocarriers comprising an immunosuppressant and an anti-IgM agent.

2. The composition of claim 1, wherein the anti-IgM agent is selected from antibodies or fragments thereof that specifically bind to CD10, CD19, CD20, CD22, CD27, CD34, CD40, CD79a, CD79b, CD123, CD179b, FLT-3, ROR1, BR3, BAFF, or B7RP-1; tyrosine kinase inhibitors; PI3K inhibitors; PKC inhibitors; APRIL antagonists; mizoribine; tofacitinib; and tetracyclines.

3. The composition of claim 2, wherein the anti-IgM agent is an anti-BAFF antibody or antigen-binding fragment thereof.

4. The composition of claim 2, wherein the anti-IgM agent is a BTK inhibitor.

5. The composition of claim 1, wherein the viral transfer vector is a retroviral transfer vector, an adenoviral transfer vector, a lentiviral transfer vector or an adeno-associated viral transfer vector.

6. The composition of claim 5, wherein the viral transfer vector is an adenoviral transfer vector, and the adenoviral transfer vector is a subgroup A, subgroup B, subgroup C, subgroup D, subgroup E, or subgroup F adenoviral transfer vector.

7. The composition of claim 5, wherein the viral transfer vector is a lentiviral transfer vector, and the lentiviral transfer vector is an HIV, SIV, FIV, EIAV or ovine lentiviral vector.

8. The composition of claim 5, wherein the viral transfer vector is an adeno-associated viral transfer vector, and the adeno-associated viral transfer vector is an AAV1, AAV2, AAV5, AAV6, AAV6.2, AAV7, AAV8, AAV9, AAV10 or AAV11 adeno-associated viral transfer vector.

9. The composition of claim 1, wherein the viral transfer vector is a chimeric viral transfer vector.

10. The composition of claim 9, wherein the chimeric viral transfer vector is an AAV-adenoviral transfer vector.

11. The composition of claim 1, wherein the transgene of the viral transfer vector comprises a gene therapy transgene, a gene editing transgene, an exon skipping transgene or a gene expression modulating transgene.

12. The composition of claim 1, wherein the synthetic nanocarriers comprise lipid nanoparticles, polymeric nanoparticles, metallic nanoparticles, surfactant-based emulsions, dendrimers, buckyballs, nanowires, virus-like particles or peptide or protein particles.

13-18. (canceled)

19. The composition of claim 1, wherein the mean of a particle size distribution obtained using dynamic light scattering of a population of the synthetic nanocarriers is a diameter greater than 110 nm.

20-37. (canceled)

38. The composition of claim 1, wherein the immunosuppressant is an inhibitor of the NF-kB pathway.

39. The composition of claim 1, wherein the immunosuppressant is an mTOR inhibitor.

40. The composition of claim 1, wherein the immunosuppressant is a rapalog.

41. (canceled)

42. The composition of claim 1, wherein an aspect ratio of a population of the synthetic nanocarriers is greater than 1:1, 1:1.2, 1:1.5, 1:2, 1:3, 1:5, 1:7 or 1:10.

43. A kit comprising the composition of claim 1 and instructions for use.

44-45. (canceled)

46. A method comprising:
establishing an anti-viral transfer vector attenuated response in a subject by concomitant administration of a viral transfer vector, synthetic nanocarriers comprising an immunosuppressant, and an anti-IgM agent to the subject.

47. (canceled)

48. A method comprising:
escalating transgene expression of a viral transfer vector in a subject by repeatedly, concomitantly administering to the subject a viral transfer vector, synthetic nanocarriers comprising an immunosuppressant, and an anti-IgM agent.

49-59. (canceled)
